(19) United States
(12) Patent Application Publication
(10) Pub. No.: US 2022/0276235 A1
(43) Pub. Date: Sep. 1, 2022

(54) IMAGING BASED HOMOGENEOUS ASSAY

(71) Applicant: Essenlix Corporation, Monmouth Junction, NJ (US)

(72) Inventors: Stephen Y. CHOU, Princeton, NJ (US); Wei DING, Princeton, NJ (US); Ji LI, Princeton, NJ (US)

(73) Assignee: Essenlix Corporation, Monmouth Junction, NJ (US)

(21) Appl. No.: 17/627,877

(22) PCT Filed: Sep. 18, 2020
(86) PCT No.: PCT/US2020/051658

§ 371 Date: Jan. 18, 2022

Related U.S. Application Data
(60) Provisional application No. 62/875,960, filed on Jul. 18, 2019.

(51) Int. Cl. G01N 33/543; G06V 20/69; G06V 10/70; G06V 10/60

(52) U.S. Cl. CPC G01N 33/54313; G01N 33/54393; G01N 33/54373; G06V 20/693; G06V 10/70; G06V 20/698; G06V 10/60; H04M 1/215

(57) ABSTRACT

Among other things, the present disclosure provides devices and methods for improving a homogeneous assay, particularly in improving accuracy, reduce noises, none-perfect conditions, multiplexing, etc.

### CROSS-REFERENCE TO RELATED APPLICATIONS

This application is a National Stage entry (§ 371) application of International Application No. PCT/US2020/051658, filed on Sep. 18, 2020, which claims the benefit of priority of U.S. Provisional Patent Application No. 62/875,960, filed Jul. 18, 2019, the contents of which are relied upon and incorporated herein by reference in their entirety. The entire disclosure of any publication or patent document mentioned herein is entirely incorporated by reference.

### FIELD

The present disclosure is related to the field of bio/chemical sampling, sensing, assays and applications. Particularly, the present invention is related to bio/chemical assays, including, including immunoassays and nucleic acid assays.

### BACKGROUND

In rapid biological and chemical assays (e.g., diagnostic testing), a homogeneous assay, which does not comprise a wash step, is preferred. The present disclosure provides devices and methods for improving a homogeneous assay, particularly in improving accuracy, reduce noises, none-perfect conditions, multiplexing, etc.

### SUMMARY

The present invention is related to, among other things, improve performance of a homogeneous assay, particularly in improving accuracy, reduce noises, none-perfect conditions, multiplexing, etc.

Homogeneous assay has several issues that desire for better solution. The homogeneous sandwich assay has Hock effect. The homogeneous competitive assay will be dark at high analyte concentration, which can confuse with other situations. One of the biggest challenge in a simple rapid assay is that there are many none-perfect factors that can give false signal (e.g. dust can scattering light to confuse a real signal. The present invention provides solutions to these issues.

One aspect of the present invention is that (i) to sandwich a sample and a bead(s) into a thin sample layer, and use of bead(s) to capture/concentrate the relevant bioagent/biomarker on the beads, (ii) taking, without washing the sample, at least two images, including a first image and a second image, of a common area of the sample layer, wherein the common area of the sample layer is an area of the sample that contains the bead, wherein the first image is a direct image for measuring topology of the sample including a position and geometry of the bead; and the second image is a signal image for measuring a signal from the labeled detection agent, and (iii) (g) comparing and analyzing, using an algorithm, the first image and the second image to identify a signal from a labeled detection agent that is attached to the bead.

Other aspect of the present invention is that the first image is bright field image and the second image is a fluorescence and/or other luminescence image.

Other aspect of the present invention is that it can reduce or eliminate the optical noise (e.g. scattered light or false signal) created by none-perfect sample factors, namely none-ideal conditions, (e.g. dust, air bubble, debris, etc.).

Other aspect of the present invention is that it can significantly improve the signal by machine learning.

Other aspect of the present invention is that it can significantly improve the signal by machine learning by using spacer as a reference.

Other aspect of the present invention is that it can significantly improve the signal by machine learning and the machine learning includes the none-ideal conditions.

Other aspect of the present invention is that it uses a single set of bead to perform both sandwich assay and competitive assay in parallel in the same assay test using the same sample.

Other aspect of the present invention is that it uses two sets of beads to perform both sandwich assay and competitive assay in parallel in the same assay test using the same sample.

Other aspect of the present invention is that it multiplexing to test several different analyte in the same sample in parallel in a single run by multiple either sandwich assays, competitive assays or both.

Other aspect of the present invention is that for a sample holder with two movable plates, in the open configuration, the beads are on the same plate that the spacers are fixed on. This arrangement can reduce the damage to the beads in operation.

In some embodiments, the present disclosure provides a method for performing a homogeneous assay of an analyte in a sample, including providing a sample that contains or is suspected of containing an analyte, providing one bead, providing a capture agent and a labeled detection agent, providing a sample holder that is configured to make at least a part of the sample into a sample layer of a thickness of 200 um or less, having the sample in the sample holder, wherein the bead and the labeled detection agent are mixed with the sample in the sample layer, taking, without washing the sample, at least two images, including a first image and a second image, of a common area of the sample layer, wherein the common area of the sample layer is an area of the sample that contains the bead, wherein the first image is a direct image for measuring topology of the sample including a position and geometry of the bead; and the second image is a signal image for measuring a signal from the labeled detection agent, and comparing and analyzing, using an algorithm, the first image and the second image to identify a signal from a labeled detection agent that is attached to the bead, wherein the capture agent is attached onto the surface of the bead, and binds to the analyte or the labeled detection agent and wherein the labeled detection agent binds to the capture agent or the analyte.

In some embodiments, the present disclosure provides an apparatus for performing a homogeneous assay of an analyte in a sample, including a capture agent, a labeled detection agent, a sample holder that is configured to make at least a part of the sample into a sample layer of a thickness of 200 um or less, an imager that is configured to take at least two images, including a first image and a second image, of a common area of the sample layer, wherein the common area of the sample layer is an area of the sample that contains the bead, wherein the first image is a direct image for measuring topology of the sample including a position and geometry of the bead, and the second image is a signal image for measuring a signal from the labeled detection agent, a computer readable medium that contain an algorithm that compares and analyzes, using an algorithm, the first image and the second image to identify a signal from a labeled detection agent that is attached to the bead, wherein the capture agent is attached onto the surface of the bead, and bines to the analyte or the labeled detection agent, and wherein the labeled detection agent gives a signal and binds to the capture agent or the analyte.

In some embodiments, the thickness of the sample layer and the diameter of the bead are selected, so that when there are more than one beads, the beads do not substantially overlap with each other in a direction normal to the sample layer such that when viewing from the top of the sample layer, no bead substantially blocks a view of any other bead.

In some embodiments, the first image is bright field image.

In some embodiments, the second image is a fluorescence and/or other luminescence image.

In some embodiments, the second image is a dark field image.

In some embodiments, the signal is an optical signal.

In some embodiments, the capture agent binds only to the analyte, and the labeled detection agent binds only to the analyte.

In some embodiments, the capture agent binds to both the analyte and the labeled detection agent, and the labeled detection agent binds only the capture agent.

In some embodiments, the capture agent binds to both the labeled detection agent, and the labeled detection agent binds to both the analyte and the capture agent.

In some embodiments, the algorithm use an image of the spacer in the first image and/or the second image.

In some embodiments, the label detection agent has a label that is selected from the group consisting of a fluorescent label, a colorimetric label, and luminescent label.

In some embodiments, the beads have various shape and have a maximum dimension in the range of 0.05 um to 50 um.

In some embodiments, the sample holder is configured make the sample layer having uniform thickness.

In some embodiments, the sample holder comprising a first plate and a second plate that are movable relative to each other into different configurations, including an operation and a closed configuration, wherein in the open configuration the first plate and second plate are at least partially separated such that the sample can be deposited on one or both plates, and wherein the closed configuration is configured after the sample deposition in the open configuration, and in the closed configuration: the first plate and the second plate confine at least a portion of the sample between the plates into a layer having a thickness of 200 um or less.

In some embodiments, the sample holder includes a first plate and a second plate that are movable relative to each other into different configurations, including an operation and a closed configuration, one or both of plates are flexible, and spacers that have a uniform height of 200 um or less, and are fixed on one of the plates, wherein in the open configuration the first plate and second plate are at least partially separated and the spacing between the two plate are not regulated by the spacers, such that the sample can be deposited t on one or both plates, and wherein the closed configuration is configured after the sample deposition in the open configuration, and in the closed configuration: at least part of the deposited sample is confined by the two plates into a thin layer that has a substantially uniform thickness, the substantially uniform thickness is regulated by the plates and the spacers.

In some embodiments, the present disclosure provides A kit for performing a homogeneous assay for analyzing an analyte in a sample including a bead, a capture agent, a labeled detection agent, a sample holder that is configured to make at least a part of the sample into a sample layer of a thickness of 200 um or less, wherein the capture agent is attached onto the surface of the bead, and bines to the analyte or the labeled detection agent, wherein the labeled detection agent gives a signal and binds to the capture agent or the analyte, wherein sample holder comprising a first plate and a second plate that are movable relative to each other into different configurations, including an operation and a closed configuration, wherein in the open configuration the first plate and second plate are at least partially separated such that the sample can be deposited on one or both plates, and wherein the closed configuration is configured after the sample deposition in the open configuration, and in the closed configuration: the first plate and the second plate confine at least a portion of the sample between the plates into a layer having a thickness of 200 um or less.

In some embodiments, the present disclosure provides a programed imager for performing a homogeneous assay for analyzing an analyte in a sample, including an imager that is configured to take at least two images, including a first image and a second image, of a common area of the sample layer, wherein the common area of the sample layer is an area of the sample that contains the bead, wherein the first image is a direct image for measuring topology of the sample including a position and geometry of the bead; and the second image is a signal image for measuring a signal from the labeled detection agent, a computer readable medium that contain an algorithm that compares and analyzes, using an algorithm, the first image and the second image to identify a signal from a labeled detection agent that is attached to the bead, wherein the capture agent is attached onto the surface of the bead, and bines to the analyte or the labeled detection agent, and wherein the labeled detection agent gives a signal and binds to the capture agent or the analyte.

In some embodiments, the first image is bright field image.

In some embodiments, the second image is a dark field image.

In some embodiments, the second image is a dark field image, and the signal is a fluorescence and/or other luminescence signal.

In some embodiments, the signal is an optical signal.

In some embodiments, the labeled detection agent binds to the analyte, but not to the capture agent.

In some embodiments, the labeled detection agent binds the capture agent, but not to the analyte.

In some embodiments, the algorithm use an image of the spacer in the first image and/or the second image.

In some embodiments, the label detection agent has a label that is selected from the group consisting of a fluorescent label, a colorimetric label, and luminescent label.

In some embodiments, the algorithm is machine learning.

In some embodiments, the algorithm is machine learning and wherein the machine learning utilizes a property of the spacers.

In some embodiments, the algorithm is machine learning and wherein the machine learning utilizes a property of the beads.

In some embodiments, the algorithm is machine learning and wherein the machine learning analyze air bubble, dust, breakage, other non-sample factors or any combination in the sample layer.

In some embodiments, the bead includes more than one beads, wherein the beads are arranged to make the beads not substantially overlapping with each other in a direction normal to the sample layer, such that when viewing from the top of the sample layer, no bead substantially blocks a view of any other bead.

In some embodiments, the thickness of the sample layer and the concentration of the labeled detection agent are selected, so that the labeled detection agent attached to the capture agent on the bead is distinguishable from signal emanating from other area in the layer of uniform thickness.

In some embodiments, in an open configuration, the beads are on the same plate that the spacers are fixed.

In some embodiments, the spacer height is the same as the maximum size of a bead (e.g. diameter) and is 15 um or less.

In some embodiments, the spacer height is the same as the maximum size of a bead (e.g. diameter) and is 10 um.

In some embodiments, the present disclosure provides a second set of capture agent and labeled detection agent, wherein the second capture agent is attached on the bead and captures a second analyte in the sample or the second labeled detection agent, and the second labeled detection agent binds to the second capture agent or the second analyte, and wherein the second analyte is bio/chemically different analyte from the first analyte in the sample. In some embodiments, the present disclosure provides more than one set of capture agent and labeled detection agent, wherein each set of capture agent is attached on the bead and captures a corresponding analyte in the sample or the labeled detection agent, and each set of labeled detection agent binds to the corresponding capture agent or the corresponding analyte, and wherein each set of analyte is bio/chemically different analyte from other set of analyte in the sample.

In some embodiments, the present disclosure provides a second set of capture agent and labeled detection agent, and a second set of bead, wherein the second capture agent is attached on the second set of bead and captures a second analyte in the sample or the second set of labeled detection agent, and the second labeled detection agent binds to the second capture agent or the second analyte, and wherein the second analyte is bio/chemically different analyte from the first analyte in the sample and the second set of bead has a different property from the first set of beads.

In some embodiments, the present disclosure provides more than one set of capture agent and labeled detection agent, and more than one set of beads, wherein each set of capture agent is attached on each corresponding set of bead and captures a corresponding analyte in the sample or the labeled detection agent, and each set of labeled detection agent binds to the corresponding capture agent or the corresponding analyte, and wherein each set of analyte is bio/chemically different analyte from other set of analyte in the sample, and each set of bead has a different property from other set of beads.

In some embodiments, different set of the labeled detection agent has a different property respect each other.

In some embodiments, different set of the labeled detection agent has a different property respect each other, including different optical spectrum.

In some embodiments, the present disclosure provides more than one set of capture agent and labeled detection agent, wherein each set of capture agent is attached on the bead and captures a corresponding analyte in the sample or the labeled detection agent, wherein at least one set of labeled detection agent binds only to the corresponding analyte, and wherein each set of analyte is bio/chemically different analyte from other set of analyte in the sample.

In some embodiments, the present disclosure provides more than one set of capture agent and labeled detection agent, and more than one set of beads, wherein each set of capture agent is attached on each corresponding set of bead and captures a corresponding analyte in the sample or the labeled detection agent, wherein at least one set of labeled capture agent binds only to the corresponding set of labeled detection agent, and wherein each set of analyte is bio/chemically different analyte from other set of analyte in the sample, and each set of bead has a different property from other set of beads.

In some embodiments, the apparatus and methods include a combination of all prior claims.

In some embodiments, the capture agent includes a molecule, protein, nucleic acid, or aptemer.

In some embodiments, the labeled detection agent includes a molecule, protein, nucleic acid, or aptemer.

In some embodiments, the concentration of the analyte is measured by measuring the signal on the bead(s).

In some embodiments, the concentration of the analyte is measured by measuring the signal on the bead(s) and measuring the signal in the sample layer but away from the bead(s).

In some embodiments, the beads have a capture agent attached on their surface and have a maximum size of 0.2 um to 100 um;

In some embodiments, an algorithm to identify the signal at the beads.

In some embodiments, the beads are randomly distributed in the thin sample layer.

In some embodiments, the total assay time is less than 10 sec, 20 sec, 30 sec, 40 sec, 50 sec, 60 sec, 120 sec, 180 sec, 240 sec, 300 sec, 400 sec, or 500 sec.

In some embodiments, the beads have a diameter in a range of 1 μm to 10 μm, or 10 μm to 50 μm.

In some embodiments, the beads or beads can be made of polystyrene, polypropylene, polycarbonate, glass, metal or any other material whose surface can be modified to bind antibodies.

In some embodiments, the diameter of the beads is no larger than the pillar height.

In some embodiments, the diameter of the beads about the same as the pillar height.

In some embodiments, the present disclosure provides a smartphone system for a homogeneous assay, including a device of any prior embodiment, a mobile communication device that includes one or a plurality of cameras for detecting and/or imaging the sample, electronics, signal processors, hardware and software for receiving and/or processing the detected signal and/or the image(s) of the sample and for remote communication, and an adaptor that is configured to accommodate the device that is in the closed configuration and be engageable to the mobile communication device, wherein when engaged with the mobile communication device, the adaptor is configured to facilitate the detection and/or imaging of the analyte in the sample, and wherein the imager takes, at least two images, including a first image and a second image, of a common area of the thin sample layer, wherein the common area of the thin sample layer is an area of the sample that contains at least one bead, wherein the first image is a direct image for measuring a position of a bead in the common area; and the second image is a signal image for measuring a signal from the labeled competitive detection agent.

In some embodiments, the first image and the second image, each includes multiple images.

In some embodiments, the spacer or the beads are arranged periodically.

In some embodiments, the first and second beads are different in their optical properties selected from the group consisting of: photoluminescence, electroluminescence, and electrochemiluminescence, light absorption, reflection, transmission, diffraction, scattering, diffusion, surface Raman scattering, and any combination thereof.

In some embodiments, the labeled detection agent is coated on one or both of the plates, and is configured to, upon contacting the sample, be dissolved and diffuse in the sample.

In some embodiments, the labeled detection agent is pre-loaded into the sample before the sample is deposited on the plate(s).

In some embodiments, wherein the beads have an average diameter in the range of 0.1 μm to 10 μm.

In some embodiments, the analyte is selected from the group consisting of: molecules, cells, viruses, proteins, peptides, DNAs, RNAs, nucleic acid, nanoparticles, and any combination thereof.

In some embodiments, the capture agent is a protein.

In some embodiments, the capture agent is a nucleic acid.

In some embodiments, the labeled detection agent is a protein.

In some embodiments, the labeled detection agent is a nucleic acid.

In some embodiments, the beads are made of a material selected from the group consisting of: polystyrene, polypropylene, polycarbonate, PMMA, PC, COC, COP, glass, resin, aluminum, gold or other metal or any other material whose surface can be modified to be associated with the capture agent.

In some embodiments, the liquid sample is made from a biological sample selected from the group consisting of: amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, urine, and any combination thereof.

In some embodiments, the sample is an environmental liquid sample from a source selected from the group consisting of: river, lake, pond, ocean, glaciers, icebergs, rain, snow, sewage, reservoirs, tap water, or drinking water, solid samples from soil, compost, sand, rocks, concrete, wood, brick, sewage, and any combination thereof.

In some embodiments, the sample is an environmental gaseous sample from a source selected from the group consisting of: the air, underwater heat vents, industrial exhaust, vehicular exhaust, and any combination thereof.

In some embodiments, the sample is a foodstuff sample selected from the group consisting of: raw ingredients, cooked food, plant and animal sources of food, preprocessed food, and partially or fully processed food, and any combination thereof.

In some embodiments, the detection agent is labeled with a fluorophore.

In some embodiments, the beads are associated with a label, and wherein the detection agent is a quencher that is configured to quench signal of the beads-associated label when the detection agent is in proximity of the label.

In some embodiments, the signal is luminescence selected from the group consisting of photoluminescence, electroluminescence, and electrochemiluminescence, light absorption, reflection, transmission, diffraction, scattering, or diffusion, surface Raman scattering; and any combination thereof.

In some embodiments, the method further includes determining the presence of the analyte and/or measuring the amount of the analyte.

In some embodiments, the one or more beads have a maximum dimension in the range of 0.05 um to 30 um.

In some embodiments, the thickness of the sample is 0.1 um, 0.5 um, 1 um, 2 um, 3 um, 4 um, 5 um, 10 um, 15 um, 20 um, 25 um, 30 um, 50 um, or a range between any two values thereof.

In some embodiments, the spacer height is equal to the diameter of the beads.

In some embodiments, the algorithm use an image of the spacer in the first image and/or the second image.

In some embodiments, the calculated parameters include average signal intensity from all the beads that are analyzed.

In some embodiments, the calculated parameters include highest signal intensity from all the beads that are analyzed.

In some embodiments, the calculated parameters include signal intensity distribution from all the beads that are analyzed.

In some embodiments, the calculated parameters include number of all the beads that are analyzed with signal intensity larger than a threshold.

In some embodiments, the calculated parameters include average signal intensity from all the beads that are analyzed in a first area of the image.

In some embodiments, the calculated parameters include highest signal intensity from all the beads that are analyzed in a first area of the image.

In some embodiments, the calculated parameters include signal intensity distribution from all the beads that are analyzed in a first area of the image.

In some embodiments, the calculated parameters include number of all the beads that are analyzed in a first area of the image with signal intensity larger than a threshold.

In some embodiments, the common area of the sample is an area of the sample comprising at least one of the one or more beads.

In some embodiments, one of the two or more images is a signal image.

In some embodiments, the beads one or more beads do not substantially overlap each other in a direction normal to the layer having uniform thickness.

In some embodiments, the device includes two plates and spacers, and wherein the inter spacer distance is periodic.

In some embodiments, the device includes two plates and spacers, and wherein the inter spacer distance (SD) is equal or less than about 120 um (micrometer).

In some embodiments, the device includes two plates and spacers, and wherein the inter spacer distance (SD) is equal or less than about 100 um (micrometer).

In some embodiments, the device includes two plates and spacers, and wherein the fourth power of the inter-spacer-distance (ISD) divided by the thickness (h) and the Young's modulus (E) of the flexible plate (ISD\hat{ }4/(hE)) is 5×10\hat{ }6 um\hat{ }3/GPa or less.

In some embodiments, the device includes two plates and spacers, and wherein the fourth power of the inter-spacer-distance (ISD) divided by the thickness (h) and the Young's modulus (E) of the flexible plate (ISD\hat{ }4/(hE)) is 5×10\hat{ }5 um3/GPa or less.

In some embodiments, the device includes two plates and spacers, and wherein the fourth power of the inter-spacer-distance (ISD) divided by the thickness (h) and the Young's modulus (E) of the flexible plate (ISD\hat{ }4/(hE)) is 5×10\hat{ }5 um3/GPa or less, the thickness of the flexible plate times the Young's modulus of the plate is 150-600 GPa, and the spacer is periodic.

In some embodiments, the device includes two plates and spacers, and wherein the spacers have pillar shape, a substantially flat top surface, a predetermined substantially uniform height, and a predetermined constant inter-spacer distance that is at least about 2 times larger than the size of the analyte, wherein the Young's modulus of the spacers times the filling factor of the spacers is equal or larger than 2 MPa, wherein the filling factor is the ratio of the spacer contact area to the total plate area, and wherein, for each spacer, the ratio of the lateral dimension of the spacer to its height is at least 1 (one).

In some embodiments, the device includes two plates and spacers, and wherein the spacers have pillar shape, a substantially flat top surface, a predetermined substantially uniform height, and a predetermined constant inter-spacer distance that is at least about 2 times larger than the size of the analyte, wherein the Young's modulus of the spacers times the filling factor of the spacers is equal or larger than 2 MPa, wherein the filling factor is the ratio of the spacer contact area to the total plate area, and wherein, for each spacer, the ratio of the lateral dimension of the spacer to its height is at least 1 (one), wherein the fourth power of the inter-spacer-distance (ISD) divided by the thickness (h) and the Young's modulus (E) of the flexible plate (ISD\hat{ }4/(hE)) is 5×10\hat{ }6 um\hat{ }3/GPa or less.

In some embodiments, the device includes two plates and spacers, and wherein the ratio of the inter-spacing distance of the spacers to the average width of the spacer is 2 or larger, and the filling factor of the spacers multiplied by the Young's modulus of the spacers is 2 MPa or larger.

In some embodiments, the spacers have a shape of pillars and a ratio of the width to the height of the pillar is equal or larger than one.

In some embodiments, the sample is the sample in the detection of proteins, peptides, nucleic acids, synthetic compounds, inorganic compounds.

In some embodiments, the sample is the sample in the fields of human, veterinary, agriculture, foods, environments, and drug testing.

In some embodiments, the sample is a biological sample selected from the group consisting of blood, serum, plasma, a nasal swab, a nasopharyngeal wash, saliva, urine, gastric fluid, spinal fluid, tears, stool, mucus, sweat, earwax, oil, a glandular secretion, cerebral spinal fluid, tissue, semen, vaginal fluid, interstitial fluids derived from tumorous tissue, ocular fluids, spinal fluid, a throat swab, breath, hair, finger nails, skin, biopsy, placental fluid, amniotic fluid, cord blood, lymphatic fluids, cavity fluids, sputum, pus, microbiota, meconium, breast milk, exhaled condensate nasopharyngeal wash, nasal swab, throat swab, stool samples, hair, finger nail, ear wax, breath, connective tissue, muscle tissue, nervous tissue, epithelial tissue, cartilage, cancerous sample, and bone.

In some embodiments, the analyte is morphine.

In some embodiments, the present disclosure provides a method of imaging objects on QMAX card in both bright-field illumination and fluorescence illumination, including inserting the QMAX card comprising the sample into the optical reader, turning on LED light on the smartphone to illuminate on the observing spot on the QMAX card from its back side, turning on the camera of smartphone, adjusting the lens position of camera of the smartphone to make the sample on QMAX card focused, taking an image with proper exposure setting, turning off the LED of smartphone and keep smartphone camera on, turning on the laser diode, adjusting the lens position of camera of the smartphone to make the sample on QMAX card focused, and taking an image.

In some embodiments, both the bright field signal and fluorescence images are taken within a time frame of 0.5 to 1.0 second

In some embodiments, the fluorescence taking parameters are ISO 800 to 1600, and integration time ⅓ s to 1 s.

In some embodiments, the bright field signal taking parameters are ISO 400 to 800, and integration time 1/200 s to 1/50 s.

In some embodiments, the present disclosure provides an optical system for observing objects on a QMAX card using bright field and fluorescence, the optical system including a smartphone and an optical reader.

In some embodiments, the optical reader includes a lens, a receptacle slot that is configured to receive and position the QMAX card in a sample slide in the field of view and focal range of the camera of smartphone, bright-field illumination optics that are configured to capture bright-field images of the sample on the QMAX card, and fluorescent illumination optics that are configured to capture fluorescent images of the sample on the QMAX card;

In some embodiments, the bright-field illumination optics include an LED light source, where in the LED light source is from the smartphone or an individual light source, and a pair of 45-degree mirrors, wherein the pair of 45-degree mirrors are disposed underneath the QMAX card, and deflect the light from the LED light source to illuminate the observing spot on the QMAX card from its back side.

In some embodiments, the fluorescence illumination optics includes an emission filter, a laser diode light source, an excitation filter, a mirror, and a lens, wherein the mirror deflects the laser light beam to illuminate on the observing spots on the QMAX card from its back side with a light incident angle to the card of 5 degree, 10 degree, 15 degree, 20 degree, 25 degree, or in a range between any of the two values, wherein the central wavelength of the laser diode can be a 405 nm, 450 nm, 525 nm, 532 nm, 635 nm, 638 nm; and the output optical power can be 10 mW, 20 mW, 30 mW, 50 mW, 100 mW, 150 mW, 200 mW, or in a range between any of the two values, wherein the excitation filter is at the front of the laser diode to clean up the excitation light, and wherein the emission filter is between the lens and smartphone camera to block the excitation laser light and to allow the fluorescence signal to go through.

In some embodiments, the fluorescence illumination optics of the optical system includes an emission filter, a laser diode light source, an excitation filter, a mirror, a lens, and a pair of polarizers, wherein the mirror deflects the laser light beam to illuminate on the observing spots on the QMAX card from its back side with a light incident angle to the card of 5 degree, 10 degree, 15 degree, 20 degree, 25 degree, or in a range between any of the two values, wherein the central wavelength of the laser diode can be a 405 nm, 450 nm, 525 nm, 532 nm, 635 nm, 638 nm; and the output optical power can be 10 mW, 20 mW, 30 mW, 50 mW, 100 mW, 150 mW, 200 mW, or in a range between any of the two values, wherein the excitation filter is at the front of the laser diode to clean up the excitation light, wherein the emission filter is between the lens and smartphone camera to block the excitation laser light and to allow the fluorescence signal to go through, and wherein the first polarizer was between the laser diode and the excitation filter, or between the excitation filter and mirror, or between mirror and QMAX card, and the second polarizer is between the lens and the QMAX card, and the orientation of the polarizer is tuned to make the polarization of the one polarizer perpendicular to that of the other.

In some embodiments, the focal length of the lens can be 1 mm, 2 mm, 4 mm, 6 mm, 10 mm, 20 mm, 30 mm, or in a range between any two values thereof.

In some embodiments, the excitation filter can be a 650 nm short pass filter with the use of a laser diode with central wavelength of 638 nm.

In some embodiments, the emission filter can be a 670 nm long pass filter with the use of a laser diode with central wavelength of 638 nm.

In some embodiments, the present disclosure provides a method of imaging objects on QMAX card in bright-filed illumination including inserting the QMAX card comprising the sample into the optical reader, turning on an LED light on the smartphone to illuminate on the observing spot on the QMAX card from its back side, turning on the camera of smartphone, adjusting the lens position of camera of the smartphone to make the sample on QMAX card focused, and taking an image.

In some embodiments, the device or system includes a first mirror and a second mirror.

In some embodiments, the device or system includes one mirror with a tilted angle between 20 degree to 40 degree to reflect the LED light on the back of QMAX card.

In some embodiments, the device or system includes a third mirror.

In some embodiments, the laser diode directly illuminate on the QMAX card from its back side with a light incident angle to the card between 5 degree to 20 degree.

In some embodiments, the present disclosure further includes a focus lens between the QMAX card and mirror 1 to magnify the field of view of bright field.

In some embodiments, the lens has a focus distance of 4 mm to 6 mm and a numerical aperture of 0.1 to 0.3 and 1 to 4 mm away underneath the QMAX card.

In some embodiments, the QMAX card reader (or adapter) reads both the bright field signal and fluorescence signal at the same spot of a QMAX card within a time frame of 0.5 to 1.0 second.

In some embodiments, the smartphone LED, mirror 1 and mirror 2 are all replaced by an external LED directly underneath the QMAX card.

In some embodiments, the capture agent is selected from the group consisting of: protein, peptide, peptidomimetics, streptavidin, biotin, oligonucleotide, oligonucleotide mimetics, any other affinity ligand and any combination thereof.

In some embodiments, the sample is related to infectious and parasitic disease, injuries, cardiovascular disease, cancer, mental disorders, neuropsychiatric disorders, pulmonary diseases, renal diseases, and other and organic diseases.

In some embodiments, the samples are related to the detection, purification and quantification of microorganism.

In some embodiments, the sample is related to virus, fungus and bacteria from environment, e.g., water, soil, or biological samples.

In some embodiments, the sample is related to the detection, quantification of chemical compounds or biological samples that pose hazard to food safety or national security, e.g. toxic waste, anthrax.

In some embodiments, the samples are related to quantification of vital parameters in medical or physiological monitor.

In some embodiments, the samples are related to glucose, blood, oxygen level, total blood count.

In some embodiments, the samples are related to the detection and quantification of specific DNA or RNA from biosamples.

In some embodiments, the samples are related to the sequencing and comparing of genetic sequences in DNA in the chromosomes and mitochondria for genome analysis.

In some embodiments, the samples are related to detect reaction products, e.g., during synthesis or purification of pharmaceuticals.

In some embodiments, the first and second beads are different in their sizes.

In some embodiments, the first and second beads are different in their electric densities.

In some embodiments, the first and second beads are the same, and wherein the signals from the first and second analytes are different.

In some embodiments, the present disclosure provides a smartphone system for rapid homogeneous assay, including any device from the foregoing embodiments, a mobile communication device that includes one or a plurality of cameras for detecting and/or imaging the sample, electronics, signal processors, hardware and software for receiving and/or processing the detected signal and/or the image of the sample and for remote communication, and an adaptor that is configured to hold the closed device and engageable to mobile communication device, wherein when engaged with the mobile communication device, the adaptor is configured to facilitate the detection and/or imaging of the analyte in the sample at the closed configuration.

In some embodiments, the intended assay time is in the range of 0.1-240 sec.

In some embodiments, the intended assay time is in the range of 1-60 sec.

In some embodiments, the intended assay time is equal to or less than 30 sec.

In some embodiments, the intended assay time is equal to or less than 10 sec.

In some embodiments, the intended assay time is equal to or less than 5 sec.

In some embodiments, the intended assay time is equal to or less than 1 sec.

In some embodiments, the average distance between two neighboring analyte concentration areas or beads is in the range of 50 nm-200 um.

In some embodiments, the average distance between two neighboring analyte concentration areas or beads is in the range of 500 nm-20 um.

In some embodiments, the average distance between two neighboring analyte concentration areas or beads is in the range of 500 nm-10 um.

In some embodiments, the average distance between two neighboring analyte concentration areas or beads is in the range of 500 nm-5 um.

In some embodiments, the ratio of the spacers' height versus the diffusion parameter is in the range of 0.01-2.

In some embodiments, the ratio of the spacers' height versus the diffusion parameter is in the range of 0.1-1.5.

In some embodiments, the ratio of the spacers' height versus the diffusion parameter is in the range of 0.01-0.5.

In some embodiments, the ratio of the spacers' height versus the diffusion parameter is in the range of 0.01-0.2.

In some embodiments, the ratio of the spacers' height versus the diffusion parameter is in the range of 0.01-0.1.

In some embodiments, the ratio of the average distance between two neighboring analyte concentration areas or beads versus the diffusion parameter is in the range of 0.01-5.

In some embodiments, the ratio of the average distance between two neighboring analyte concentration areas or beads versus the diffusion parameter is in the range of 0.01-1.5.

In some embodiments, the ratio of the average distance between two neighboring analyte concentration areas or beads versus the diffusion parameter is in the range of 0.01-1.

In some embodiments, the ratio of the average distance between two neighboring analyte concentration areas or beads versus the diffusion parameter is in the range of 0.01-0.5.

In some embodiments, the ratio of the average distance between two neighboring analyte concentration areas or beads versus the diffusion parameter is in the range of 0.01-0.2.

In some embodiments, the ratio of the average distance between two neighboring analyte concentration areas or beads versus the diffusion parameter is in the range of 0.01-0.1

In some embodiments, the ratio of the average distance between two neighboring analyte concentration areas or beads versus the diffusion parameter is in the range of 0.01-0.5, and the ratio of the spacers' height versus the diffusion parameter is in the range of 0.01-0.2.

In some embodiments, the ratio of the average distance between two neighboring analyte concentration areas or beads versus the diffusion parameter is in the range of 0.01-1, and the ratio of the spacers' height versus the diffusion parameter is in the range of 0.01-0.5.

In some embodiments, the ratio of the average distance between two neighboring analyte concentration areas or beads versus the diffusion parameter is in the range of 0.01-2, and the ratio of the spacers' height versus the diffusion parameter is in the range of 0.01-1. In some embodiments, the ratio of the average distance between two neighboring analyte concentration areas or beads versus the diffusion parameter is in the range of 0.01-4, and the ratio of the spacers' height versus the diffusion parameter is in the range of 0.01-1.

In some embodiments, the ratio of the average distance between two neighboring analyte concentration areas or beads versus the diffusion parameter is in the range of 0.01-0.5, the ratio of the spacers' height versus the diffusion parameter is in the range of 0.01-0.2, and the intended assay time is equal to or less than 120 sec.

In some embodiments, the ratio of the average distance between two neighboring analyte concentration areas or beads versus the diffusion parameter is in the range of 0.01-1; the ratio of the spacers' height versus the diffusion parameter is in the range of 0.01-0.5, and the intended assay time is equal to or less than 60 sec.

In some embodiments, the ratio of the average distance between two neighboring analyte concentration areas or beads versus the diffusion parameter is in the range of 0.01-2; the ratio of the spacers' height versus the diffusion parameter is in the range of 0.01-1; and the intended assay time is equal to or less than 30 sec.

In some embodiments, the ratio of the average distance between two neighboring analyte concentration areas or beads versus the diffusion parameter is in the range of 0.01-4; the ratio of the spacers' height versus the diffusion parameter is in the range of 0.01-1; and the intended assay time is equal to or less than 30 sec.

In some embodiments, the analyte is C Reactive Protein (CRP).

In some embodiments, ratio between the spacing between the plates at the closed configuration and average dimeter of the beads is in the range of 1-100.

In some embodiments, one or both of the plates includes a signal amplification surface that amplify the signal in proximity to the amplification surface.

In some embodiments, the beads and the detection agent are on different plates.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

A skilled artisan will understand that the drawings, described below, are for illustration purposes only. In some Figures, the drawings are in scale. In the figures that present experimental data points, the lines that connect the data points are for guiding a viewing of the data only and have no other means. For clarity purposes, some elements are enlarged when illustrated in the Figures. It should be noted that the Figures do not intend to show the elements in strict proportion. The dimensions of the elements should be delineated from the descriptions herein provided and incorporated by reference. The drawings are not intended to limit the scope of the present invention in any way.

FIG. 1 illustrates a schematic view of a sandwich assay with the sample holder, beads, reagents, and the imager that takes two images: First image: Topology of sample (e.g. Bright field), Second image: Signal of labeled detection agent (e.g. Fluorescence)

FIG. 2 illustrates a schematic view of a competitive assay with the sample holder, beads, reagents, and the imager

FIG. 3 illustrates the first image (bright field) and the second image (fluorescence image) of the sample location of a sample layer that is inside a sample holder for a sandwich assay. The imperfection (i.e. none-ideal factors: dust, debris, etc.) that are clearly identify in the bright field give fluorescence signal in the fluorescence image.

FIG. 4 illustrates a cross-sectional view of an exemplary system for homogeneous assay with two movable plates and spacers, at an open configuration and a closed configuration. In the open configuration, the beads are on the same plate that the spacers are fixed on. This arrangement can reduce the damage to the beads in operation.

FIG. 5 illustrates a schematic of top view of a homogeneous assay by local concentration according to one embodiment of the present invention. Signal from the beads and from the background are measured. In some embodiment, the signal of the beads are measured by removing the effects of the background signals.

FIG. 6 illustrates a schematic view of a homogeneous assay by local concentration according to one embodiment of the present invention. The capture agents are coated on the sidewall of the spacer of a pillar shape. With a Ti/Si coating on top of the pillar and the surface of the plate, only the pillar sidewall can be coated with capture agent, while without the Ti/Si coating, the capture agent coats everywhere. The images shows that for the capture agent coated only on the sidewall of the spacer gives a stronger fluorescence signal.

FIG. 8 illustrates a schematic view of an amplification by a single molecule assay according to one embodiment of the present invention.

FIG. 9 illustrates an example of a QMAX card reader (or adapter), which reads both the bright field signal and fluorescence signal at the same spot of a QMAX card.

FIG. N3 illustrates a schematic view of a homogeneous assay by local concentration according to one embodiment of the present invention.

FIG. N4 illustrates a pillar array structures fabricated on a QMAX card for use with the homogeneous assay by local concentration according to one embodiment of the present invention.

FIG. N5 illustrates a C-reactive protein (CRP) homogeneous assay by local concentration according to one embodiment of the present invention.

FIG. 7 illustrates a graph displaying fluorescence intensity versus CRP concentration of two separate CRP immunoassay performed. In one immunoassay the QMAX card was coated with a Ti/Si anti-capture agent layer and the other immunoassay was not coated with the same. The results demonstrate that the fluorescence signal of the immunoassay is better when coated with the Ti/Si than not.

### DETAILED DESCRIPTION

The following detailed description illustrates certain embodiments of the invention by way of example and not by way of limitation. If any, the section headings and any subtitles used herein are for organizational purposes only and are not to be construed as limiting the subject matter described in any way. The contents under a section heading and/or subtitle are not limited to the section heading and/or subtitle, but apply to the entire description of the present invention.

### Definitions

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The terms “labeled analyte” and “bound label” are interchangeable. The phrase “labeled analyte” refers to an analyte that is detectably labeled with a light emitting label such that the analyte can be detected by assessing the presence of the label. A labeled analyte may be labeled directly (e.g., the analyte itself may be directly conjugated to a label, e.g., via a strong bond, e.g., a covalent or non-covalent bond), or a labeled analyte may be labeled indirectly (e.g., the analyte is bound by a secondary capture agent that is directly labeled).

The terms “unbound label” and “background” are interchangeable, with understanding that the signal of “unbound label” includes signals from other background that are not “unbound label”.

The term “lateral area” refers to the area that is in parallel with the plate.

The term “analyte-concentration area” refers to an area of a surface where the area has a higher affinity to bind the labeled analyte/bound label (or to bind an analyte what later binds a label) than the rest area of the surface.

The term “lateral distance between two neighboring analyte concentration areas” or “IACD (inter analyte concentration-area distance)” refers to the distance between the average center of each analyte concentration area. For example, if each of the analyte concentration area has a circular shape in lateral shape, the IACD is the distance between the centers of the two circles. Another example, if each of the two analyte concentration areas is a vertical plane, then the IACD is the lateral distance between the two planes.

The term “diffusion parameter” or “DP” as used herein refers to a parameter that is equal to √Dt, wherein D is the diffusion constant of the analyte in the sample and the t is the intended assay time (e.g., the diffusion parameter is equal to the square-root of the diffusion constant of the analyte in the sample multiplying the intended assay time); wherein the intended assay time is a time parameter. For example, if the diffusion constant of the analyte in the sample is 1×10-7 cm2/s, the intended assay time is 60 sec, then the diffusion parameter is 24 um (micron). Some of the common analyte diffusion constants are IgG in PBS: 3×10-7 cm2/s, IgG in blood: 1×10-7 cm2/s, and 20 bp DNA in blood: 4×10-7 cm2/s.

The term “bead” as used herein refers to a nano-scale or micro-scale three-dimensional object, regardless of its shape and material. The term “bead” and “particle” is interchangeable.

The term “specifically capture” means that a capture agent selectively bound an analyte that will be detected.

The term “compressed open flow (COF)” refers to a method that changes the shape of a flowable sample deposited on a plate by (i) placing other plate on top of at least a part of the sample and (ii) then compressing the sample between two plates by pushing the two plates towards each other; wherein the compression reduces a thickness of at least a part of the sample and makes the sample flow into open spaces between the plates.

The term “compressed regulated open flow” or “CROF” (or “self-calibrated compressed open flow”) refers to a particular type of COF, wherein the final thickness of a part or entire sample after the compression is “regulated” by spacers, wherein the spacers, that are placed between the two plates.

The terms “specific binding” and “selective binding” refer to the ability of a capture agent to preferentially bind to a particular target molecule that is present in a heterogeneous mixture of different target molecule. A specific or selective binding interaction will discriminate between desirable (e.g., active) and undesirable (e.g., inactive) target molecules in a sample, typically more than about 10 to 100-fold or more (e.g., more than about 1000- or 10,000-fold).

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “polynucleotide”, “nucleotide”, “nucleotide sequence”, “nucleic acid”, “nucleic acid molecule”, “nucleic acid sequence” and “oligonucleotide” are used interchangeably, and can also include plurals of each respectively depending on the context in which the terms are utilized. They refer to a polymeric form of nucleotides of any length, either deoxyribonucleotides (DNA) or ribonucleotides (RNA), or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA, ribozymes, small interfering RNA, (siRNA), microRNA (miRNA), small nuclear RNA (snRNA), cDNA, recombinant polynucleotides, branched polynucleotides, plasmids, vectors, isolated DNA (A, B and Z structures) of any sequence, PNA, locked nucleic acid (LNA), TNA (treose nucleic acid), isolated RNA of any sequence, nucleic acid probes, and primers. LNA, often referred to as inaccessible RNA, is a modified RNA nucleotide. The ribose moiety of an LNA nucleotide is modified with an extra bridge connecting the 2′ and 4′ carbons. The bridge “locks” the ribose in the 3′-endo structural conformation, which is often found in the A-form of DNA or RNA, which can significantly improve thermal stability.

The term “capture agent” as used herein, refers to a binding member, e.g. nucleic acid molecule, polypeptide molecule, or any other molecule or compound, that can specifically bind to its binding partner, e.g., a second nucleic acid molecule containing nucleotide sequences complementary to a first nucleic acid molecule, an antibody that specifically recognizes an antigen, an antigen specifically recognized by an antibody, a nucleic acid aptamer that can specifically bind to a target molecule, etc. A capture agent may concentrate the target molecule from a heterogeneous mixture of different molecules by specifically binding to the target molecule. Binding may be non-covalent or covalent. The affinity between a binding member and its binding partner to which it specifically binds when they are specifically bound to each other in a binding complex is characterized by a KD (dissociation constant) of 10-5 M or less, 10-6 M or less, such as 10-7 M or less, including 10-8 M or less, e.g., 10-9 M or less, 10-10 M or less, 10-11 M or less, 10-12 M or less, 10-13 M or less, 10-14 M or less, 10-15 M or less, including 10-16 M or less. “Affinity” refers to the strength of binding, increased binding affinity being correlated with a lower KD.

The term “a secondary capture agent” which can also be referred to as a “detection agent” refers a group of biomolecules or chemical compounds that have highly specific affinity to the antigen. The secondary capture agent can be strongly linked to an optical detectable label, e.g., enzyme, fluorescence label, or can itself be detected by another detection agent that is linked to an optical detectable label through bioconjugation (Hermanson, “Bioconjugate Techniques” Academic Press, 2nd Ed., 2008).

The term “capture agent-reactive group” refers to a moiety of chemical function in a molecule that is reactive with capture agents, e.g., can react with a moiety (e.g., a hydroxyl, sulfhydryl, carboxyl or amine group) in a capture agent to produce a stable strong, e.g., covalent bond.

The term “antibody,” as used herein, is meant a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (σ), and alpha (α) which encode the IgM, IgD, IgG, IgE, and IgA antibody “isotypes” or “classes” respectively. Antibody herein is meant to include full length antibodies and antibody fragments, and may refer to a natural antibody from any organism, an engineered antibody, or an antibody generated recombinantly for experimental, therapeutic, or other purposes. The term “antibody” includes full length antibodies, and antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies.

The terms “antibody epitope,” “epitope,” “antigen” are used interchangeably herein to refer to a biomolecule that is bound by an antibody. Antibody epitopes can include proteins, carbohydrates, nucleic acids, hormones, receptors, tumor markers, and the like, and mixtures thereof. An antibody epitope can also be a group of antibody epitopes, such as a particular fraction of proteins eluted from a size exclusion chromatography column. Still further, an antibody epitope can also be identified as a designated clone from an expression library or a random epitope library.

An “allergen,” as used herein is a substance that elicits an allergic, inflammatory reaction in an individual when the individual is exposed to the substance, e.g., by skin contact, ingestion, inhalation, eye contact, etc. An allergen may include a group of substances that together elicit the allergic reaction. Allergens may be found in sources classified by the following groups: natural and artificial fibers (cotton, linen, wool, silk, teak, etc., wood, straw, and other dust); tree pollens (alder, birch, hazel, oak, poplar, palm, and others); weeds and flowers (ambrosia, artemisia, and others); grasses and corns (fescue, timothy grass, rye, wheat, corn, bluegrass, and others); drugs (antibiotics, antimicrobial drugs, analgetics and non-steroid anti-inflammatory drugs, anesthetics and muscle relaxants, hormones, and others); epidermal and animal allergens (epithelium of animals, feathers of birds, sera, and others); molds and yeasts (Penicillium notation, Cladosporium spp., Aspergillus fumigatus, Mucor racemosus, and others); insect venoms; preservatives (butylparaben, sorbic acid, benzoate, and others); semen (ejaculate); parasitic and mite allergens (ascarids, Dermatophagoides pteronyssinus, Dermatophagoides farinae, Euroglyphus maynei, and others); occupational and hobby allergens (coffee beans, formaldehyde, latex, chloramine, dyes, and others); food allergens (egg products, dairy products and cheeses, meat products, fish and seafood, soy products, mushrooms, flours and cereals, vegetables, melons and gourds, beans, herbs and spices, nuts, citrus and other fruits, berries, teas and herbs, nutritional supplements, and other products), etc.

The term “Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these.

As is known to one skilled in the art, hybridization can be performed under conditions of various stringency. Suitable hybridization conditions are such that the recognition interaction between a capture sequence and a target nucleic acid is both sufficiently specific and sufficiently stable. Conditions that increase the stringency of a hybridization reaction are widely known and published in the art. See, for example, Green, et al., (2012), infra.

The term “protein” refers to a polymeric form of amino acids of any length, e.g., greater than 2 amino acids, greater than about 5 amino acids, greater than about 10 amino acids, greater than about 20 amino acids, greater than about 50 amino acids, greater than about 100 amino acids, greater than about 200 amino acids, greater than about 500 amino acids, greater than about 1000 amino acids, greater than about 2000 amino acids, usually not greater than about 10,000 amino acids, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like. Also included by these terms are polypeptides that are post-translationally modified in a cell, e.g., glycosylated, cleaved, secreted, prenylated, carboxylated, phosphorylated, etc., and polypeptides with secondary or tertiary structure, and polypeptides that are strongly bound, e.g., covalently or non-covalently, to other moieties, e.g., other polypeptides, atoms, cofactors, etc.

The term “complementary” as used herein refers to a nucleotide sequence that base-pairs by hydrogen bonds to a target nucleic acid of interest. In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. Typically, “complementary” refers to a nucleotide sequence that is fully complementary to a target of interest such that every nucleotide in the sequence is complementary to every nucleotide in the target nucleic acid in the corresponding positions. When a nucleotide sequence is not fully complementary (100% complementary) to a non-target sequence but still may base pair to the non-target sequence due to complementarity of certain stretches of nucleotide sequence to the non-target sequence, percent complementarily may be calculated to assess the possibility of a non-specific (off-target) binding. In general, a complementary of 50% or less does not lead to non-specific binding. In addition, a complementary of 70% or less may not lead to non-specific binding under stringent hybridization conditions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 200 nucleotides and up to 300 nucleotides in length, or longer, e.g., up to 500 nucleotides in length or longer. Oligonucleotides may be synthetic and, in certain embodiments, are less than 300 nucleotides in length.

The term “attaching” as used herein refers to the strong, e.g., covalent or non-covalent, bond joining of one molecule to another.

The term “surface attached” as used herein refers to a molecule that is strongly attached to a surface.

The term “sample” as used herein relates to a material or mixture of materials containing one or more analytes or entity of interest. In particular embodiments, the sample may be obtained from a biological sample such as cells, tissues, bodily fluids, and stool. Bodily fluids of interest include but are not limited to, amniotic fluid, aqueous humour, vitreous humour, blood (e.g., whole blood, fractionated blood, plasma, serum, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine and exhaled condensate. In particular embodiments, a sample may be obtained from a subject, e.g., a human, and it may be processed prior to use in the subject assay. For example, prior to analysis, the protein/nucleic acid may be extracted from a tissue sample prior to use, methods for which are known. In particular embodiments, the sample may be a clinical sample, e.g., a sample collected from a patient.

The term “analyte” refers to a molecule, cells, tissues, viruses, and nanoparticles with different shapes, and wherein the molecule comprising a protein, peptide, DNA, RNA, nucleic acid, or other molecule.

The term “assaying” refers to testing a sample to detect the presence and/or abundance of an analyte.

As used herein, the terms “determining,” “measuring,” and “assessing,” and “assaying” are used interchangeably and include both quantitative and qualitative determinations.

As used herein, the term “light-emitting label” refers to a label that can emit light when under an external excitation. This can be luminescence. Fluorescent labels (which include dye molecules or quantum dots), and luminescent labels (e.g., electro- or chemi-luminescent labels) are types of light-emitting label. The external excitation is light (photons) for fluorescence, electrical current for electroluminescence and chemical reaction for chemi-luminescence. An external excitation can be a combination of the above.

The terms “hybridizing” and “binding”, with respect to nucleic acids, are used interchangeably.

The term “capture agent/analyte complex” is a complex that results from the specific binding of a capture agent with an analyte. A capture agent and an analyte for the capture agent will usually specifically bind to each other under “specific binding conditions” or “conditions suitable for specific binding”, where such conditions are those conditions (in terms of salt concentration, pH, detergent, protein concentration, temperature, etc.) which allow for binding to occur between capture agents and analytes to bind in solution. Such conditions, particularly with respect to antibodies and their antigens and nucleic acid hybridization are well known in the art (see, e.g., Harlow and Lane (Antibodies: A Laboratory Manual Cold Spring Harbor Laboratory, Cold Spring Harbor, N.Y. (1989) and Ausubel, et al, Short Protocols in Molecular Biology, 5th ed., Wiley & Sons, 2002).

The term “specific binding conditions” and “conditions suitable for binding,” as used herein with respect to binding of a capture agent to an analyte, e.g., a biomarker, a biomolecule, a synthetic organic compound, an inorganic compound, etc., refers to conditions that produce nucleic acid duplexes or, protein/protein (e.g., antibody/antigen) complexes, protein/compound complexes, aptamer/target complexes that contain pairs of molecules that specifically bind to one another, while, at the same time, disfavor to the formation of complexes between molecules that do not specifically bind to one another. Specific binding conditions are the summation or combination (totality) of both hybridization and wash conditions, and may include a wash and blocking steps, if necessary. For nucleic acid hybridization, specific binding conditions can be achieved by incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 ug/ml denatured, sheared salmon sperm DNA, followed by washing the filters in 0.1×SSC at about 65° C.

For binding of an antibody to an antigen, specific binding conditions can be achieved by blocking a first plate containing antibodies in blocking solution (e.g., PBS with 3% BSA or non-fat milk), followed by incubation with a sample containing analytes in diluted blocking buffer. After this incubation, the first plate is washed in washing solution (e.g. PBS+TWEEN 20) and incubated with a secondary capture antibody (detection antibody, which recognizes a second site in the antigen). The secondary capture antibody may be conjugated with an optical detectable label, e.g., a fluorophore such as IRDye800CW, Alexa 790, Dylight 800. After another wash, the presence of the bound secondary capture antibody may be detected. One of skill in the art would be knowledgeable as to the parameters that can be modified to increase the signal detected and to reduce the background noise.

A subject may be any human or non-human animal. A subject may be a person performing the instant method, a patient, a customer in a testing center, etc.

An “analyte,” as used herein is any substance that is suitable for testing in the present invention.

As used herein, a “diagnostic sample” refers to any biological sample that is a bodily byproduct, such as bodily fluids, that has been derived from a subject. The diagnostic sample may be obtained directly from the subject in the form of liquid, or may be derived from the subject by first placing the bodily byproduct in a solution, such as a buffer. Exemplary diagnostic samples include, but are not limited to, saliva, serum, blood, sputum, urine, sweat, lacrima, semen, feces, breath, biopsies, mucus, etc.

As used herein, an “environmental sample” refers to any sample that is obtained from the environment. An environmental sample may include liquid samples from a river, lake, pond, ocean, glaciers, icebergs, rain, snow, sewage, reservoirs, tap water, drinking water, etc.; solid samples from soil, compost, sand, rocks, concrete, wood, brick, sewage, etc.; and gaseous samples from the air, underwater heat vents, industrial exhaust, vehicular exhaust, etc. Typically, samples that are not in liquid form are converted to liquid form before analyzing the sample with the present invention.

As used herein, a “foodstuff sample” refers to any sample that is suitable for animal consumption, e.g., human consumption. A foodstuff sample may include raw ingredients, cooked food, plant and animal sources of food, preprocessed food as well as partially or fully processed food, etc. Typically, samples that are not in liquid form are converted to liquid form before analyzing the sample with the present invention.

The term “diagnostic,” as used herein, refers to the use of a method or an analyte for identifying, predicting the outcome of and/or predicting treatment response of a disease or condition of interest. A diagnosis may include predicting the likelihood of or a predisposition to having a disease or condition, estimating the severity of a disease or condition, determining the risk of progression in a disease or condition, assessing the clinical response to a treatment, and/or predicting the response to treatment.

A “biomarker,” as used herein, is any molecule or compound that is found in a sample of interest and that is known to be diagnostic of or associated with the presence of or a predisposition to a disease or condition of interest in the subject from which the sample is derived. Biomarkers include, but are not limited to, polypeptides or a complex thereof (e.g., antigen, antibody), nucleic acids (e.g., DNA, miRNA, mRNA), drug metabolites, lipids, carbohydrates, hormones, vitamins, etc., that are known to be associated with a disease or condition of interest.

A “condition” as used herein with respect to diagnosing a health condition, refers to a physiological state of mind or body that is distinguishable from other physiological states. A health condition may not be diagnosed as a disease in some cases. Exemplary health conditions of interest include, but are not limited to, nutritional health; aging; exposure to environmental toxins, pesticides, herbicides, synthetic hormone analogs; pregnancy; menopause; andropause; sleep; stress; prediabetes; exercise; fatigue; chemical balance; etc. The term “biotin moiety” refers to an affinity agent that includes biotin or a biotin analogue such as desthiobiotin, oxybiotin, 2′-iminobiotin, diaminobiotin, biotin sulfoxide, biocytin, etc. Biotin moieties bind to streptavidin with an affinity of at least 10-8M. A biotin affinity agent may also include a linker, e.g., -LC-biotin, -LC-LC-Biotin, -SLC-Biotin or -PEGn-Biotin where n is 3-12.

The term “streptavidin” refers to both streptavidin and avidin, as well as any variants thereof that bind to biotin with high affinity.

The term “marker”, as used in describing a biological sample, refers to an analyte whose presence or abundance in a biological sample is correlated with a disease or condition.

The term “bond” includes covalent and non-covalent bonds, including hydrogen bonds, ionic bonds and bonds produced by van der Waal forces.

The term “amplify” refers to an increase in the magnitude of a signal, e.g., at least a 10-fold increase, at least a 100-fold increase at least a 1,000-fold increase, at least a 10,000-fold increase, or at least a 100,000-fold increase in a signal.

The term “entity” refers to, but not limited to proteins, peptides, DNA, RNA, nucleic acid, molecules (small or large), cells, tissues, viruses, nanoparticles with different shapes, that would bind to a “binding site”. The entity includes the capture agent, detection agent, and blocking agent. The “entity” includes the “analyte”, and the two terms are used interchangeably.

The term “binding site” refers to a location on a solid surface that can immobilize “entity” in a sample.

The term “entity partners” refers to, but not limited to proteins, peptides, DNA, RNA, nucleic acid, molecules (small or large), cells, tissues, viruses, nanoparticles with different shapes, that are on a “binding site” and would bind to the entity. The entity, include, but not limited to, capture agents, detection agents, secondary detection agents, or “capture agent/analyte complex”.

The term “target analytes” or “target entity” refers to a particular analyte that will be specifically analyzed (e.g., detected), or a particular entity that will be specifically bound to the binding site.

The term “smart phone” or “mobile phone”, which are used interchangeably, refers to the type of phones that has a camera and communication hardware and software that can take an image using the camera, manipulate the image taken by the camera, and communicate data to a remote place. In some embodiments, the Smart Phone has a flash light.

The term “light” refers to, unless specifically specified, an electromagnetic radiation with various wavelength.

The term “average linear dimension” of an area is defined as a length that equals to the area times 4 then divided by the perimeter of the area. For example, the area is a rectangle, that has width w, and length L, then the average of the linear dimension of the rectangle is 4*W*L/(2*(L+W)) (where “*” means multiply and “I” means divide). By this definition, the average line dimension is, respectively, W for a square of a width W, and d for a circle with a diameter d. The area include, but not limited to, the area of a binding site or a storage site.

The term “period” of periodic structure array refers to the distance from the center of a structure to the center of the nearest neighboring identical structure.

The term “storage site” refers to a site of an area on a plate, wherein the site contains reagents to be added into a sample, and the reagents are capable of being dissolving into the sample that is in contract with the reagents and diffusing in the sample.

The term “relevant” means that it is relevant to detection of analytes, quantification and/or control of analyte or entity in a sample or on a plate, or quantification or control of reagent to be added to a sample or a plate.

The term “hydrophilic”, “wetting”, or “wet” of a surface means that the contact angle of a sample on the surface is less than 90 degree.

The term “hydrophobic”, “non-wetting”, or “does not wet” of a surface means that the contact angle of a sample on the surface is equal to or larger than 90 degrees.

The term “variation” of a quantity refers to the difference between the actual value and the desired value or the average of the quantity. And the term “relative variation” of a quantity refers to the ratio of the variation to the desired value or the average of the quantity. For example, if the desired value of a quantity is Q and the actual value is (Q+μ), then the μ is the variation and the μ/(Q+μ) is the relative variation. The term “relative sample thickness variation” refers to the ratio of the sample thickness variation to the average sample thickness.

The term “optical transparent” refers to a material that allows a transmission of an optical signal, wherein the term “optical signal” refers to, unless specified otherwise, the optical signal that is used to probe a property of the sample, the plate, the spacers, the scale-marks, any structures used, or any combinations of thereof.

The term “none-sample-volume” or “none-sample factor” refers to, at a closed configuration of a CROF process, the volume between the plates that is occupied not by the sample but by other objects that are not the sample. The objects include, but not limited to, spacers, air bubbles, dusts, or any combinations of thereof. Often none-sample-volume(s) is mixed inside the sample.

The term “saturation incubation time” refers to the time needed for the binding between two types of molecules (e.g. capture agents and analytes) to reach an equilibrium. For a surface immobilization assay, the “saturation incubation time” refers the time needed for the binding between the target analyte (entity) in the sample and the binding site on plate surface reaches an equilibrium, namely, the time after which the average number of the target molecules (the entity) captured and immobilized by the binding site is statistically nearly constant.

In some cases, the “analyte” and “binding entity” and “entity” are interchangeable.

A “processor,” “communication device,” “mobile device,” refer to computer systems that contain basic electronic elements (including one or more of a memory, input-output interface, central processing unit, instructions, network interface, power source, etc.) to perform computational tasks. The computer system may be a general purpose computer that contains instructions to perform a specific task, or may be a special-purpose computer.

A “site” or “location” as used in describing signal or data communication refers to the local area in which a device or subject resides. A site may refer to a room within a building structure, such as a hospital, or a smaller geographically defined area within a larger geographically defined area. A remote site or remote location, with reference to a first site that is remote from a second site, is a first site that is physically separated from the second site by distance and/or by physical obstruction. The remote site may be a first site that is in a separate room from the second site in a building structure, a first site that is in a different building structure from the second site, a first site that is in a different city from the second site, etc.

As used herein, “raw data” includes signals and direct read-outs from sensors, cameras, and other components and instruments which detect or measure properties or characteristics of a sample. For example, raw data includes voltage or current output from a sensor, detector, counter, camera, or other component or device; raw data includes digital or analog numerical output from a sensor, detector, counter, camera, or other component or device; and raw data may include digitized or filtered output from a sensor, detector, counter, camera, or other component or device. For example, raw data includes the output of a luminometer, which may include output in “relative light units” which are related to the number of photons detected by the luminometer. Raw data may include a JPEG, bitmap, or other image file produced by a camera. Raw data may include cell counts; light intensity (at a particular wavelength, or at or within a range of wavelengths); a rate of change of the output of a detector; a difference between similar measurements made at two times; a number of events detected; the number of events detected within a pre-set range or that meet a pre-set criterion; the minimum value measured within a time period, or within a field of view; the maximum value measured within a time period, or within a field of view; and other data. Where sufficient, raw data may be used without further processing or analysis. In other cases, raw data may be further processed or used for further analysis related to the sample, the subject, or for other purposes.

“Representative of a sample” as used in reference to an output signal or raw data that are representative of the sample, refers to the output signal or raw data reflecting a measured property of the sample or a portion thereof, e.g., reflecting the amount of analyte of interest present in the sample. For instance, the intensity of a fluorescence signal representative of a sample may be more intense in a fluorescently labeled sample that contains more analyte of interest than the intensity of a fluorescence signal representative of a fluorescently labeled sample that contains less analyte.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible. One skilled artisan will appreciate that the present invention is not limited in its application to the details of construction, the arrangements of components, category selections, weightings, pre-determined signal limits, or the steps set forth in the description or drawings herein. The invention is capable of other embodiments and of being practiced or being carried out in many different ways.

Homogeneous Competitive Assays Using Dual Imaging

According to the present invention, an homogeneous competitive assay comprises a sample chamber with two plates that sandwich a sample suspect containing an analyte, one or more particles that have a capture agent attached to the surface of the particles, wherein the capture agent specifically bind to the analyte, and a labeled competitive detection agent, wherein the labeled competing detection agent competes with the analyte, if present, for binding to the capture agent for the analyte.

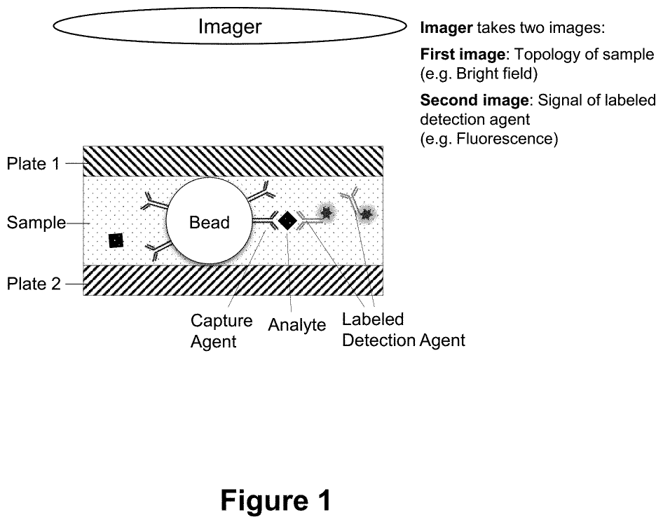

FIG. 1 illustrates a schematic view of a sandwich assay with the sample holder, beads, reagents, and the imager that takes two images: First image: Topology of sample (e.g. Bright field), Second image: Signal of labeled detection agent (e.g. Fluorescence)

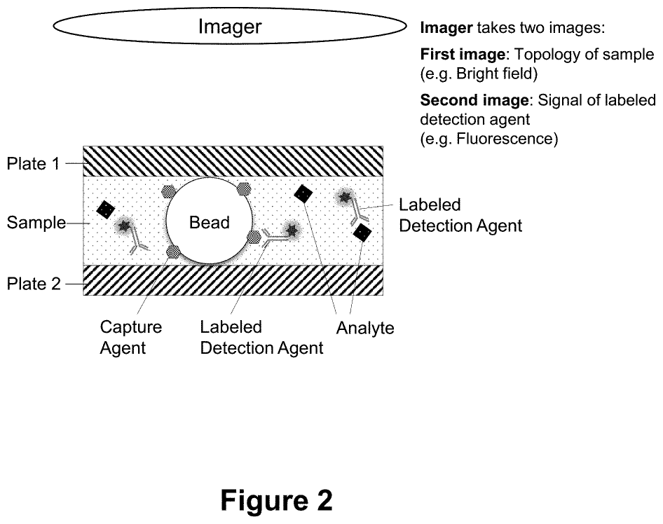

FIG. 2 illustrates a schematic view of a competitive assay with the sample holder, beads, reagents, and the imager

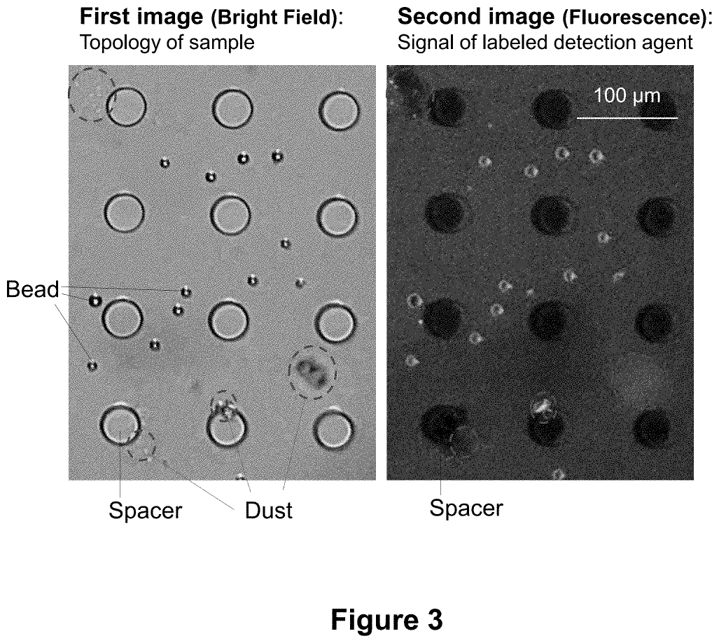

FIG. 3 illustrates the first image (bright field) and the second image (fluorescence image) of the sample location of a sample layer that is inside a sample holder for a sandwich assay. The imperfection (i.e. none-ideal factors: dust, debris, etc.) that are clearly identify in the bright field give fluorescence signal in the fluorescence image.

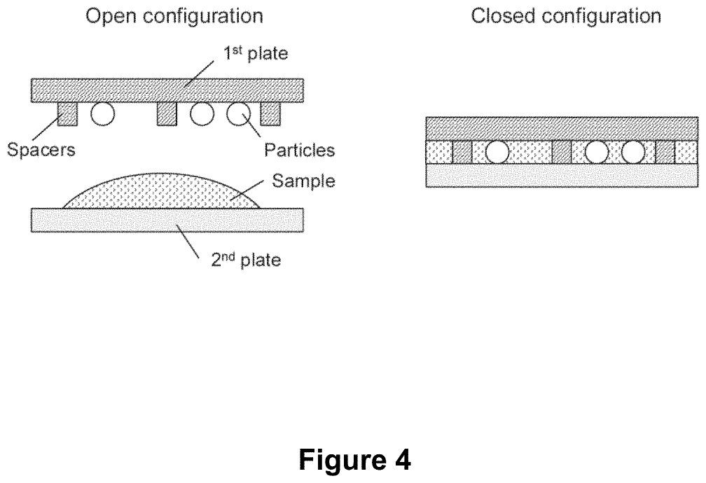

FIG. 4 illustrates a cross-sectional view of an exemplary system for homogeneous assay with two movable plates and spacers, at an open configuration and a closed configuration. In the open configuration, the beads are on the same plate that the spacers are fixed on. This arrangement can reduce the damage to the beads in operation.

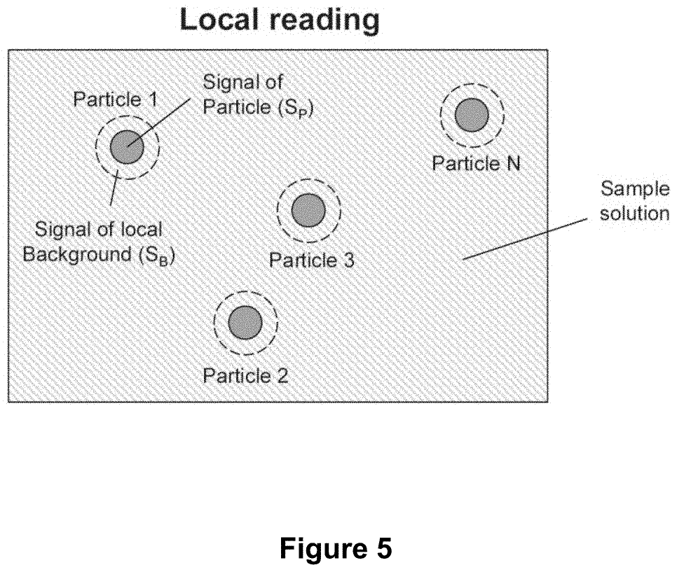

FIG. 5 illustrates a schematic of top view of a homogeneous assay by local concentration according to one embodiment of the present invention. Signal from the beads and from the background are measured. In some embodiment, the signal of the beads are measured by removing the effects of the background signals.

Using a competitive assay as an example, according to the present invention, in certain embodiments, a method for performing a competitive assay of an analyte in a liquid sample, comprising:

(a) providing a sample that contains or is suspected of containing an analyte;

(b) providing one or more beads that have a capture agent attached onto the surface of the one or more beads, wherein the capture agent specifically binds to the analyte;

(c) providing a labeled competitive detection agent, wherein the labeled competing detection agent competes with the analyte, if present, for binding to the capture agent;

(d) providing a sample holder that is configured to make the sample into a thin layer;

(e) having the sample in the sample holder and making the sample forming a thin layer having a thickness of 200 um or less, wherein the one or more beads and the labeled competitive detection agent are mixed with the sample;

(f) taking, after step (e), without washing the sample, at least two images, including a first image and a second image, of a common area of the sample layer, wherein the common area of the sample layer is an area of the sample that contains at least one bead, wherein the first image is a direct image for measuring a position of a bead in the common area; and the second image is a signal image for measuring a signal from the labeled competitive detection agent;

(g) after (f), comparing and analyzing the first image and the second image to identify the signal at the one or more beads;

wherein the beads have various shape and have a maximum dimension in the range of 0.05 um to 50 um, wherein the spacing between the inner surfaces of the two plates is configured such that in the common area (i) the sample layer has uniform thickness, and (ii) the one or more beads do not overlap with each other in a direction normal to the sample layer such that when viewing from the top of the sample layer, no bead substantially blocks a view of any other bead.

In some embodiments, a method for performing a competitive assay of an analyte in a liquid sample, comprising:

(a) providing a sample that contains or is suspected of containing an analyte;

(b) providing one or more beads that have a capture agent attached onto the surface of the beads;

(c) providing a labeled competitive detection agent, wherein the labeled competing detection agent specifically binds to the analyte and the capture agent, and wherein the capture agent competes with the analyte, if present, for binding to the labeled competing detection agent;

(d) providing a sample holder that is configured to make the sample into a thin layer having a thickness of 200 um or less;

(e) having the sample in the sample holder and making the sample forming a thin layer, wherein the beads and the labeled competitive detection agent are mixed with the sample;

(f) taking, after step (e), without washing the sample, at least two images, including a first image and a second image, of a common area of the thin sample layer, wherein the common area of the thin sample layer is an area of the sample that contains at least one bead, wherein the first image is a direct image for measuring a position of a bead in the common area; and the second image is a signal image for measuring a signal from the labeled competitive detection agent;

(g) after (f), comparing and analyzing the first image and the second image to identify the signal at the beads;

wherein the beads have various shape and have a maximum dimension in the range of 0.05 um to 50 um, wherein the spacing between the inner surfaces of the two plates is configured such that in the common area (i) the sample layer has uniform thickness, and (ii) the one or more beads do not overlap with each other in a direction normal to the sample layer such that when viewing from the top of the sample layer, no bead substantially blocks a view of any other bead.
In some embodiments, a method for assaying an analyte in a liquid using beads, comprising:
(a) depositing a sample that contains or is suspected of containing an analyte, into a sample holder, said sample holder comprising:
   i. a first plate; and
   ii. a second plate;
wherein the first plate and the second plate are movable relative to each other into:
   i. an open configuration in which the first plate and the second plate are at least partially separated such that the sample can be deposited therebetween; and
   ii. a closed configuration, in which the first plate is placed on top of the second plate thereby compressing at least a portion of the sample between the first plate and the second plate into a layer having uniform thickness of 200 um or less;
(b) having the plates into a closed configuration, wherein the sample is mixed with (i) one or more beads comprising a capture agent attached onto a surface thereof; and (ii) a labeled competitive detection agent; and
(c) taking, after step (b), while the plates are in the closed configuration and without washing the sample, at least two images, including a first image and a second image, of a common area of the sample layer, wherein the common area of the sample layer is an area of the sample that contains at least one bead, wherein the first image is a direct image for measuring a position of a bead in the common area, and wherein the second image is a signal image for measuring a signal from the labeled competitive detection agent;
(d) after (c), comparing and analyzing the first image and the second image to identify the signal at the beads;

wherein the beads have various shape and have a maximum dimension in the range of 0.05 um to 50 um, wherein the spacing between the inner surfaces of the two plates is configured such that in the common area (i) the sample layer has uniform thickness, and (ii) the one or more beads do not overlap with each other in a direction normal to the sample layer such that when viewing from the top of the sample layer, no bead substantially blocks a view of any other bead.

In some embodiments, a kit for performing a competitive assay for analyzing an analyte in a sample, comprising:

a first plate, a second plate, one or plurality of beads, a capture agent, and a labeled competing detection agent, wherein:
i. the plates are movable relative to each other into different configurations;
ii. each of the plates has, on its respective surface, a sample contact area for contacting a sample that contains an analyte;
iii. the beads have a capture agent attached onto the surface of the beads, wherein the capture agent specifically bind to the analyte;
iv. the labeled competing detection agent competes with the analyte, if present, for binding to the capture agent for the analyte;
v. beads have a capture agent attached on their surface and have a [maximum] size of 0.2 um to 100 um;

wherein one of the configurations is an open configuration, in which: the two plates are separated apart, and the sample is deposited on one or both plate;

wherein another of the configurations is a closed configuration which is configured after the sample deposition in the open configuration; and in the closed configuration: at least part of the sample is compressed by the two plates into a layer of highly uniform thickness of 200 um or less and is substantially stagnant relative to the plates; and

wherein at the closed configuration, the detector detects the analyte in the at least part of the sample.
In some embodiments, a kit for performing a competitive assay for analyzing an analyte in a sample, comprising:
a first plate, a second plate, one or plurality of beads, a capture agent, a labeled competing detection agent, and spacers wherein:
   i. the plates are movable relative to each other into different configurations;
   ii. each of the plates has, on its respective surface, a sample contact area for contacting a sample that contains an analyte;
   iii. the beads have a capture agent attached to the surface of the beads, wherein the capture agent specifically bind to the analyte;
   iv. the labeled competing detection agent competes with the analyte, if present, for binding to the capture agent for the analyte;
   v. the spacers are on one or both plate, wherein the spacers are fixed on one of the plate and has flat top, and in at least one of the spacers is in the sample area;
   vi. beads have a capture agent attached on their surface and have a size of 0.2 um to 100 um;

wherein one of the configurations is an open configuration, in which: the two plates are separated apart, the spacing between the plates is not regulated by the spacers, and the sample is deposited on one or both of the plates; and

wherein another of the configurations is a closed configuration which is configured after the sample deposition in the open configuration; and in the closed configuration: at least part of the sample is compressed by the two plates into a layer of highly uniform thickness of 200 um or less and is substantially stagnant relative to the plates, wherein the uniform thickness of the layer is confined by the inner surfaces of the two plates and is regulated by the plates and the spacers.

In some embodiments, an apparatus for analyzing an analyte in a sample, comprising:
a. an imager or imagers that is configured to take a direct illumination image and an oblique illumination image of a thin layer of a sample having a thickness of 200 um or less; wherein each of the two imagers images at least a common area of the sample, wherein the sample contains an analyte and one or plurality of beads, wherein the beads have a capture agent attached on their surface and have a size of 0.2 um to 100 um, wherein the capture agent captures the analyte, and wherein at least one of the beads is in the common area of the sample; and
b. a hardware and a software that are configured to (a) identify the common area of the sample from the direct illumination image and the oblique illumination image, (b) identify the beads in the common area of the sample in direct illumination image, (c) measure, using the info in b., the light amplitude at each pixels related to the nanobeads.

2. A method for competitive assaying an analyte in a liquid sample, comprising:

(a) providing a sample that contains or is suspected of containing an analyte;

(b) providing a sample holder of any device of prior claims;

(c) providing one or more beads that have a capture agent attached onto the surface of the beads, wherein the capture agent specifically bind to the analyte;

(d) providing a labeled competitive detection agent, wherein the labeled competing detection agent competes with the analyte, if present, for binding to the capture agent for the analyte,

(e) having the sample in the sample holder and making the sample forming a thin layer having a thickness of 200 um or less, wherein the beads and the labeled competitive detection agent are mixed with the sample;

(f) taking, after step (e), two images of a common area of the thin sample layer, wherein the common area of the sample layer is an area of the sample contains at least one bead, wherein one of the images is a direct image that comprises information of the topology (i.e. geometry) and position of the bead in the common area; and the other image is a signal image that is configured to comprises signal from the labeled competitive detection agents as a major signal of the image;

(g) after (f), comparing and analyzing the two images and using an algorithm to identify the signal at the beads;

wherein the beads have various shape and has a maximum dimension in the range of 0.05 um to 50 um, where in the spacing between the two plate inner surface is configured, so that in the thin layer of the sample, the beads do not have overlap each other in the direction in normal to the thin sample layer.

In some embodiments, a method for competitive assaying an analyte in a liquid sample, comprising:

(a) providing a sample that is suspected of containing an analyte;

(b) providing a sample holder of any device of prior claims and an apparatus of any prior claims;

(c) providing one or more beads that have a capture agent attached to the surface of the beads, wherein the capture agent specifically bind to the analyte;

(d) providing a labeled competitive detection agent, wherein the labeled competing detection agent competes with the analyte, if present, for binding to the capture agent for the analyte,

(e) having the sample in the sample holder and making the sample forming a thin layer having a thickness of 200 um or less, wherein the beads and the labeled competitive detection agent are mixed with the sample;

(f) taking, after step (e), two images of a common area of the thin sample layer, wherein the common area of the sample layer is an area of the sample contains at least one bead, wherein one of the images is a direct image that comprises information of the topology (i.e. geometry) and position of the bead in the common area; and the other image is a signal image that is configured to comprises signal from the labeled competitive detection agents as a major signal of the image;

(g) after (f), comparing and analyzing the two images and using an algorithm to identify the signal at the beads;

wherein the bead have various shape and has a maximum dimension in the range of 0.05 um to 50 um, where in the spacing between the two plate inner surface is configured, so that in the thin layer of the sample, the beads do not have overlap each other in the direction in normal to the thin sample layer.

The device, kit, apparatus, and method of any prior embodiment, wherein the direct image is bright field image.

The device, kit, apparatus, and method of any prior embodiment, wherein the direct image is an image formed with an illumination from an angle about normal to the sample thin layer (0 to 30 degree from the normal).

The device, kit, apparatus, and method of any prior embodiment, wherein the signal image is a dark field image.

The device, kit, apparatus, and method of any prior embodiment, wherein the signal image is a fluorescence image.

The device, kit, apparatus, and method of any prior embodiment, wherein the signal image is a luminescence image.

The device, kit, apparatus, and method of any prior embodiment, wherein the signal image is an image formed with an illumination from an angle about parallel to the sample thin layer (0 to 30 degree from the sample plane).

The device, kit, apparatus, and method of any prior embodiment, wherein the assay is homogeneous assay that measures the analyte does not use any no wash.

The device, kit, apparatus, and method of any prior embodiment, wherein the signal is an optical signal.

The device, kit, apparatus, and method of any prior embodiment, wherein the images have many pixels that are configured to identify the signals.

The device, kit, apparatus, and method of any prior embodiment, wherein the plate has a spacer to control the final sample thickness in measuring the signal.

The device, kit, apparatus, and method of any prior embodiment, wherein the total assay time is less than 10 sec, 20 sec, 30 sec, 40 sec, 50 sec, 60 sec, 120 sec, 180 sec, 240 sec, 300 sec, 400 sec, 500 sec, 1000 sec, or 2000 sec.

1. Bead Preparation

1.1. Anti-BSA Antibody Preparation

100 μg of anti-BSA antibody (from Rockland, Cat. 201-4133-0100) was buffer exchanged with PBS buffer using Ultracel 0.5 mL 30 k Membrane (from Millipore, Cat. UFC503008) for three times according to manufacture's protocol. 100 μg of anti-BSA antibody was finally diluted in 50 μL of PBS buffer at a concentration of 2 mg/mL.

1.2. 10 μm Bead Preparation

100 μl of 10 μm PureProteome NHS FlexiBind Magnetic Bead (from Millipore Sigma, Cat. NO. LSKMAGN04) was transferred to a 1.5 mL microcentrifuge tube. Beads were collected at the bottom of the tube using a magnet, and the storage buffer was discarded using a pipette. Beads were then immediately rinsed with ice-cold Equilibration Buffer (1 mM HCl, provided by manufacture) and vortexed vigorously for 20 s. Beads were collected at the bottom of the tube using a magnet, and the Equilibration Buffer was discarded using a pipette.

1.3. 10 μm Bead and Antibody Coupling

Immediately mixed 50 μL of anti-BSA antibody from Step 1.1 with beads from Step 1.2. Incubated beads with continuous mixing on a vortex overnight at 4° C. Beads were then collected at the bottom of the tube using a magnet and the unbounded anti-BSA antibody was removed using a pipette. Resuspended and washed the beads with 500 μL of Quench Buffer (100 mM Tris-HCl, 150 mM NaCl, pH 8.0) for three times. Incubated the beads with 500 μL of Quench Buffer at room temperature for 1 h. Beads were then washed three times with 500 μL PBS and stored in PBS at 4° C. for further use.

1.4. Morphine-BSA Competitor Coating

PBS buffer of beads from Step 1.3 was discarded with a pipette. Beads were then incubated with 50 μg of Morphine-BSA with continuous mixing on a vortex for 8 h at room temperature. Beads were collected at the bottom of the tube using a magnet and the unbounded Morphine-BSA was discarded using a pipette. Beads were then washed three times with 500 μL PBS as described above.

1.5. Blocking

Beads from Step 1.4 were incubated with 4% BSA overnight at 4° C. Beads were then washed three times with 500 μL PBS as described above, and stored in 100 μL PBS at 4° C. for further use.

2. First Plate Preparation

Beads from Step 1.5 were sonicated for 2 minutes before use. Bead density was adjusted to approximately 5 beads in each 10\hat{ }4 μm² pillar of view. 1 μL of beads was then dropped on the surface of the first plate (with 10 μm pillars), and dried in a desiccator at room temperature.

3. Second Plate Preparation

300 μg of anti-Morphine antibody (from Fitzgerald, Cat. 10-1379) was labeled with Cy5® using Abcam Cy5® fast conjugation kit according to the manufacture's protocol. The second plate, made by PMMA, was first incubated with 1% NaOH at 42° C. for 2 h, and then rinsed three times with water before incubated with 4% BSA at room temperature for 2 h. The second plate was then rinsed three times with PBS and air dried before use.

1 μL of Cy5 labeled anti-Morphine antibody was dropped and dried on the second plate in a desiccator at room temperature.

4. Morphine QMAX BEST Competitive Immunoassay

1 μL of PBS or artificial saliva spiked with certain concentrations of morphine was dropped on the first plate where beads were dried at Step 2. The first plate and the second plate were then closed into a closed configuration, and incubated for 1 min.

5. Imaging Analysis

Without washing, QMAX card was directly measured in a closed configuration using either a microscope or a smartphone. In some embodiments, the present invention takes, while the sample mixed with beads and without washing the sample, at two images of, a first image and a second image of a common area of the thin sample layer, wherein the common area of the thin sample layer is an area of the sample that contains at least one bead, wherein the first image is a direct image that measures position of a bead in the common area regardless if the bead captured a labeled competitive detection agent or not; and the second image is a signal image that is configured to measure signal from the labeled competitive detection agent. For example, the first image is a bright field image and ad the second image is fluorescence image. In some embodiments, the two type of images are taken at the same location simultaneously.

Examples of the experimental demonstration of the present inventions are given in FIGS. 2 and 19.

6. Analyte Analysis

In assaying the analytes, the analyte can be detected by either analogue means (analog BEST) or digital means (digital BEST). In analog BEST, the analyte amount in the sample is determined from the total amplitude of the light from all beads in the measurement area. While in a digital BEST, the analyte amount in the sample is determined from the number of the beads that have a light signal above a threshold value, wherein the threshold value is determined from a calibration and wherein as long as the light from a bead is equal or above the threshold it counts one bead regardless how much it is above the threshold.

In some embodiments, the background signal from the sample areas that do not have beads are measured. In some embodiments, the background signal from the none-ideal factors are measured. In some embodiments, in measuring the signal of the labeled detection agent attached to the beads, the effects of these background signal are removed from the original images.

### Certain Preferred Specifications

1. Particles (beads) can have a diameter of 100 nm, 500 nm, 1 μm, 5 μm, 50 μm, 100 μm, or a range between any two of the values; and a preferred range of 0.5 μm to 10 μm, or 10 μm to 20 μm, or 20 μm to 50 μm.
2. Particles or beads can be polystyrene, polypropylene, polycarbonate, glass, metal or any other material whose surface can be modified to bind antibodies.
3. The diameter of the beads should be no larger than the pillar height of the first plate. Preferably, the diameter of the beads is similar as the pillar height of the first plate.
4. Labels can be fluorescent, colorimetric or luminescent.
5. Sample type please refers to Homogeneous Immunoassay Provisional.
6. QMAX card please refers to Homogeneous immunoassay Provisional.

### Examples of Optical Systems

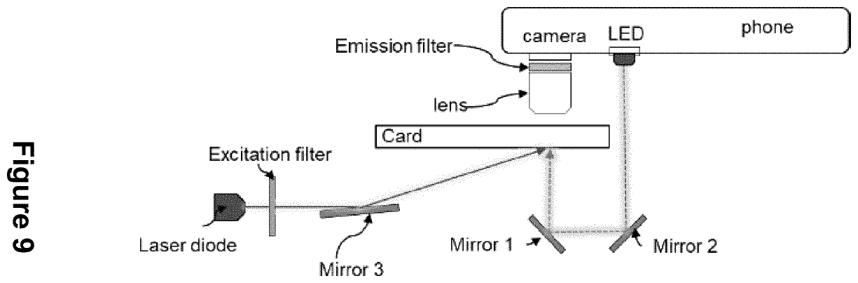

FIG. 9 illustrates an example of a QMAX card reader (or adapter), which reads both the bright field signal and fluorescence signal at the same spot of a QMAX card. In an example, the card reader uses a smartphone as both the camera and the bright field light source, and a laser diode as the fluorescence light source. In observing the bright field signal on the QMAX card, the LED light on the smartphone is reflected by two 45-degree mirrors (mirror 1 and mirror 2), which are both underneath the QMAX card, and illuminates on the observing spot on the QMAX card from its back side. The observing spot of the QMAX card is directly underneath the smartphone camera. An emission filter and a focus lens are attached at the front of the smartphone camera. In one example, the emission filter is a 670 nm long pass filter. The lens has a focus distance around 4 mm and a numerical aperture of 0.2. The typical bright field lighting up area is a circle with a diameter of 1 mm to 5 mm. The typical observing field of view for bright field is 1 mm2 to 25 mm2.

In observing the fluorescence signal on the QMAX card, the laser light from a laser diode is reflected by a mirror (mirror 3) and illuminates on the observing spots on the QMAX card from its back side with a light incident angle to the card between 5 degree to 20 degree. There is an excitation filter at the front of the laser diode to clean up the excitation light. Optional, there is an optical lens in front of the laser diode to generate line profile of the laser light. In an example, the laser diode has a 638 nm central wavelength with 120 mW power. The excitation filter is a 650 nm short pass filter. Same as the bright field, the observing spot of the QMAX card is directly underneath the smartphone camera. An emission filter and a focus lens are attached at the front of the smartphone camera. The typical fluorescence lighting up area is a square with a size of 1 mm2 to 25 mm2. The typical observing field of view for fluorescence is 1 mm2 to 25 mm2.

In observing both the bright field and fluorescence signal at the same spot of a QMAX card, the laser diode is open first, and the smartphone camera takes the fluorescence signal from the objects on the QMAX card. Immediately after the fluorescence signal is taken, the smartphone LED is open, and the smartphone camera takes the bright field signal from the objects on the QMAX card at the same spot. Typical bright field signal taking parameters are ISO 400 to 800, integration time 1/200 s to 1/50 s. Typical fluorescence taking parameters are ISO 800 to 1600, integration time ⅓ s to 1 s.

In observing both the bright field and fluorescence signal at the same spot of a QMAX card, the smartphone LED is open first, and the smartphone camera takes the bright field signal from the objects on the QMAX card. Immediately after the bright field signal is taken, the smartphone LED is closed, and the laser diode is open, and the smartphone camera takes the fluorescence signal from the objects on the QMAX card at the same spot. Typical bright field signal taking parameters are ISO 400 to 800, integration time 1/200 s to 1/50 s. Typical fluorescence taking parameters are ISO 800 to 1600, integration time ⅓ s to 1 s.

Alternatives to the Setup:

In some embodiments, mirror 1 and mirror 2 are replaced by one mirror with a tilted angle between 20 degree to 40 degree to reflect the LED light on the back of QMAX card.

In some embodiments, mirror 3 can be deleted in the setup, and the laser diode directly illuminate on the QMAX card from its back side with a light incident angle to the card between 5 degree to 20 degree.

In some embodiments, there is a focus lens between the QMAX card and mirror 1 to magnify the field of view of bright field. In an example, the lens has a focus distance of 4 mm to 6 mm and a numerical aperture of 0.1 to 0.3 and 1 to 4 mm away underneath the QMAX card.

In some embodiments, a QMAX card reader (or adapter) reads both the bright field signal and fluorescence signal at the same spot of a QMAX card within a time frame of 0.5 to 1.0 second.

In some embodiments, the smartphone LED, mirror 1 and mirror 2 are all replaced by an external LED directly underneath the QMAX card.

In some embodiments, a pair of polarizers are used. The first polarizer was put between the laser diode and the excitation filter, or between the excitation filter and mirror 3, or between mirror 3 and card. The second polarizer is between the lens and the card. The orientation of the polarizer is tuned to make the polarization of the one polarizer is perpendicular to that of the other.

In some embodiments, an optical system observing objects on card using bright field and fluorescence, comprising: a smartphone; and an optical reader. In some embodiments, the optical system of any prior embodiments, wherein the optical reader comprises: a lens; a receptacle slot that is configured to receive and position the QMAX card in a sample slide in the field of view and focal range of the camera of smartphone; a bright-field illumination optics that is configured to capture bright-field images of the sample on QMAX card; a fluorescent illumination optics that is configured to capture fluorescent images of the sample on QMAX card; In some embodiments, the optical system of any prior embodiments, wherein the bright-field illumination optics comprises. In some embodiments, a LED light source, where in the light source can be from the smartphone or an individual light source.

In some embodiments, a pair of 45-degree mirrors, wherein the two 45-degree mirrors which are both underneath the QMAX card, and deflect the light from the LED to illuminate on the observing spot on the QMAX card from its back side;

In some embodiments, the optical system of any prior embodiments, wherein the fluorescence illumination optics comprises: an emission filter; a laser diode light source; an excitation filter; a mirror; a lens; wherein the mirror deflects the laser light beam to illuminate on the observing spots on the QMAX card from its back side with a light incident angle to the card of 5 degree, 10 degree, 15 degree, 20 degree, 25 degree, or in a range between any of the two values.

In some embodiments, the central wavelength of the laser diode can be a 405 nm, 450 nm, 525 nm, 532 nm, 635 nm, 638 nm; and the output optical power can be 10 mW, 20 mW, 30 mW, 50 mW, 100 mW, 150 mW, 200 mW, or in a range between any of the two values.

wherein the excitation filter is at the front of the laser diode to clean up the excitation light;

wherein the emission filter is put between the lens and smartphone camera to block the excitation laser light and to allow the fluorescence signal to go through.

In some embodiments, the optical system of any prior embodiments, wherein the fluorescence illumination optics comprises:

an emission filter;

a laser diode light source;

an excitation filter;

a mirror;

a lens;

a pair of polarizers;

wherein the mirror deflects the laser light beam to illuminate on the observing spots on the QMAX card from its back side with a light incident angle to the card of 5 degree, 10 degree, 15 degree, 20 degree, 25 degree, or in a range between any of the two values;

wherein the central wavelength of the laser diode can be a 405 nm, 450 nm, 525 nm, 532 nm, 635 nm, 638 nm; and the output optical power can be 10 mW, 20 mW, 30 mW, 50 mW, 100 mW, 150 mW, 200 mW, or in a range between any of the two values;

wherein the excitation filter is at the front of the laser diode to clean up the excitation light;

wherein the emission filter is put between the lens and smartphone camera to block the excitation laser light and to allow the fluorescence signal to go through;

wherein the first polarizer was put between the laser diode and the excitation filter, or between the excitation filter and mirror, or between mirror and QMAX card, and the second polarizer is between the lens and the QMAX card, and the orientation of the polarizer is tuned to make the polarization of the one polarizer is perpendicular to that of the other.

In some embodiments, the optical system of any prior embodiments, wherein the focal length of the lens can be 1 mm, 2 mm, 4 mm, 6 mm, 10 mm, 20 mm, 30 mm, or in a range between any of the two values.

In some embodiments, the optical system of any prior embodiments, wherein the excitation filter can be a 650 nm short pass filter with the use of a laser diode with central wavelength of 638 nm in some embodiments, the optical system of any prior embodiments, wherein the emission filter can be a 670 nm long pass filter with the use of a laser diode with central wavelength of 638 nm.

In some embodiments, a method of imaging objects on QMAX card in bright-filed illumination, comprising:

a. Insert the QMAX card comprising the sample into the optical reader;

b. Turn on LED light on the smartphone to illuminate on the observing spot on the QMAX card from its back side;

c. Turn on the camera of smartphone;

d. adjust the lens position of camera of the smartphone to make the sample on QMAX card focused;

e. take an image with proper exposure setting.

In some embodiments, the method of imaging objects on QMAX card in fluorescence illumination, comprising:

a. Insert the QMAX card comprising the sample into the optical reader;

b. Turn on the laser diode light source;

c. Turn on the camera of smartphone;

d. adjust the lens position of camera of the smartphone to make the sample on QMAX card focused;

e. take an image with proper exposure setting.

11. A method of imaging objects on QMAX card in both bright-field illumination and fluorescence illumination, comprising:

a. Insert the QMAX card comprising the sample into the optical reader;

b. Turn on LED light on the smartphone to illuminate on the observing spot on the QMAX card from its back side;

c. Turn on the camera of smartphone;

d. adjust the lens position of camera of the smartphone to make the sample on QMAX card focused;

e. take an image with proper exposure setting.

f. Turn off the LED of smartphone and keep smartphone camera on;

g. Turn on the laser diode;

h. adjust the lens position of camera of the smartphone to make the sample on QMAX card focused;

i. take an image with proper exposure setting.

12. The method of embodiment 11, wherein both the bright field signal and fluorescence images are taken within a time frame of 0.5 to 1.0 secon

13. The method of any of prior embodiments, wherein the typical fluorescence taking parameters are ISO 800 to 1600, integration time ⅓ s to 1 s.

14. The method of any of prior embodiments, wherein the typical bright field signal taking parameters are ISO 400 to 800, integration time 1/200 s to 1/50 s.

### Alternatives to the Setup:

In some embodiments, mirror 1 and mirror 2 are replaced by one mirror with a tilted angle between 20 degree to 40 degree to reflect the LED light on the back of QMAX card.

In some embodiments, mirror 3 can be deleted in the setup, and the laser diode directly illuminate on the QMAX card from its back side with a light incident angle to the card between 5 degree to 20 degree.

In some embodiments, there is a focus lens between the QMAX card and mirror 1 to magnify the field of view of bright field. In an example, the lens has a focus distance of 4 mm to 6 mm and a numerical aperture of 0.1 to 0.3 and 1 to 4 mm away underneath the QMAX card.

In some embodiments, a QMAX card reader (or adapter) reads both the bright field signal and fluorescence signal at the same spot of a QMAX card within a time frame of 0.5 to 1.0 second.

In some embodiments, the smartphone LED, mirror 1 and mirror 2 are all replaced by an external LED directly underneath the QMAX card.

### Homogeneous Non-Competitive Assays Using Local Amplification

In certain embodiments, a homogeneous non-competitive assay competitive assay can comprise a sample holder that is configured to make a sample suspected having an analyte into a thin layer. In certain embodiments, in a homogeneous non-competitive assay competitive assay one capture surface of the sample holder can have a capture agent that specifically captures an analyte in a sample, and one non-capture surface that does not have the capture agent, wherein the capture by the capture agent is by binding to one part of the analyte. In certain embodiments, a homogeneous non-competitive assay competitive assay can comprise a labeled detection agent that specifically captures an analyte, wherein the capture by the detection agent is by binging to another part of the analyte. In certain embodiments, a homogeneous non-competitive assay competitive assay can comprise a capture surface that is to configured to amplify the optical signal of the detection agent, wherein the amplification is by one or any combination of the following: (a) directly amplifying the label optical signal using metallic structures (i.e. plasmonic structures), including micro and nanostructures, or metal/dielectric mixtures; (b) putting a light emitters (e.g. fluorophore) that emit the same or similar wavelength range of light as the labeled detection agent on or near the capture area; or (c) any combination of (a) and (b). The amplification makes the capture area brighter than the non-capture area to overcome some background signal in a homogeneous assay.

### EXAMPLES

### 1. Principles and Certain Examples

One objective of the present invention is to perform a homogeneous assay in “one step”. The “one step” assay means that in assaying, one drops a sample on the assay and then reads the signal, and there are no other steps in between (e.g. washing). The assays include, but not limited to, protein assays and nucleic acid assays.

Another objective of the present invention is to perform a “one step” assay in a time frame of about 60 seconds or less. The time is defined as the time from a sample touching the assay plate to the signal of the assay being ready to be read.

The present invention is to allow performing a homogeneous assay in “one-step” without using any washing, often being completed in about 60 seconds or less. In the “one-step” assay, it uses two plates that are movable relative to each other, a sample with an analyte is dropped on one or both of the plates, the two plates are pressed against each other to compress at least a portion of the sample into a thin layer, followed by reading the signal from the plate without any washing. Often the time, from the sample touching one of the plates to reading the signal from the plate is about 60 sec or less.

Another important feature of the present invention is that in certain embodiments, the two plates of the assay are pressed by human hands, and by using particular set of the plates and the spacers, as specified herein, at least a portion of the sample have a uniform thickness.

### Examples

According to one embodiment of the present invention, as shown in FIG. 1, a device for a homogeneous assay, comprising:

a first plate, a second plate, spacers, a plurality of particles, and capture agents, wherein:
i. the first and second plates are movable relative to each other into different configurations, including an open configuration and a closed configuration;
ii. each of the plates has, on its respective inner surface, a sample contact area for contacting a sample suspected of containing a analyte;
iii. the first plate comprises the spacers that are fixed on its inner surface, at least one of the spacers is inside the sample contact area, the spacers have a predetermined substantially uniform height that is equal to 100 um or less;
iv. the plurality of particles has the capture agents immobilized on their surface, wherein the capture agents are capable of specifically binding and immobilizing the analyte; and
v. the plurality of particles are (a) distributed on the sample contact area of the first plate, except the areas occupied by the spacers, and (b) are temporarily or permanently fixed on the first plate;

wherein in the open configuration, the two plates are partially or entirely separated apart, the spacing between the plates is not regulated by the spacers, and the sample is deposited on one or both of the plates; and

wherein in the closed configuration, which is configured after deposition of the sample in the open configuration: at least part of the sample is compressed by the two plates into a layer of highly uniform thickness, the uniform thickness of the layer is confined by the inner surfaces of the plates and is regulated by the plates and the spacers.

According to one embodiment of the present invention, a device for a homogeneous assay, comprising:

a first plate, a second plate, spacers, a plurality of particles, and capture agents, wherein:
i. the first and second plates are movable relative to each other into different configurations, including an open configuration and a closed configuration;
ii. each of the plates has, on its respective inner surface, a sample contact area for contacting a sample suspected of containing a analyte;
iii. one or both plates comprises the spacers that are fixed on its inner surface, at least one of the spacers is inside the sample contact area, the spacers have a predetermined substantially uniform height that is equal to 100 um or less;
iv. the plurality of particles has the capture agents immobilized on their surface, wherein the capture agents are capable of specifically binding and immobilizing the analyte; and
v. the plurality of particles are (a) distributed on a sample contact area of the first, and (b) are temporarily or permanently fixed on the plate;

wherein in the open configuration, the two plates are partially or entirely separated apart, the spacing between the plates is not regulated by the spacers, and the sample is deposited on one or both of the plates; and

wherein in the closed configuration, which is configured after deposition of the sample in the open configuration: at least part of the sample is compressed by the two plates into a layer of highly uniform thickness, the uniform thickness of the layer is confined by the inner surfaces of the plates and is regulated by the plates and the spacers.

Another objective of the present invention is to perform homogeneous assays accurately by (1) measuring the total optical signal for an particle area and the total optical signal from its neighboring area, and by (2) averaging several pairs of the particle area and its surrounding area.

According to one embodiment of the present invention, a method of performing a homogeneous assay, comprising the steps of:

(a) obtaining a sample suspected of containing an analyte;

(b) obtaining a device of any prior embodiment, wherein the capture agents are capable of specifically binding an binding site of the analyte;

(c) having optical labels on at least a part of the sample contact areas of the device, wherein the optical labels are capable of binding to the analytes;

(d) depositing the sample on one or both of the plates when the plates are in an open configuration, wherein in an open configuration;

(e) after (d), bringing the two plates together and pressing the plates into a closed configuration, at least part of the sample is compressed by the two plates into a layer of highly uniform thickness, the uniform thickness of the layer is confined by the inner surfaces of the plates and is regulated by the plates and the spacers;

(f) while the plates are in the closed configuration, analyzing the analyte in the layer of uniform thickness, wherein the analyzing comprises:

i. measuring, from outside of the sample layer, the total light signal from (a) a particle area that is an area of the sample layer that contains one particle and from (b) a surrounding area that is the area of the sample layer which is around the particle area, wherein the surrounding area is 50 D within the edge of the particle, wherein the D is the diameter of the particle; and

ii. measuring the total light signal from each of the particle area and the surrounding area of at least two different particle areas.

According to one embodiment of the present invention, an apparatus for homogeneous assaying an analyte in a sample, comprising:

i. a device of any prior embodiment,

ii. an imager or imagers that images at least a part of the sample contact area.

According to one embodiment of the present invention, a smartphone system for homogeneous assay, comprising:
(a) a device of any prior embodiment;
(b) a mobile communication device that comprises:
   i. one or a plurality of cameras for detecting and/or imaging the sample;
   ii. electronics, signal processors, hardware and software for receiving and/or processing the detected signal and/or the image(s) of the sample and for remote communication; and
(c) an adaptor that is configured to accommodate the device that is in the closed configuration and be engageable to the mobile communication device;

wherein when engaged with the mobile communication device, the adaptor is configured to facilitate the detection and/or imaging of the analyte in the sample.

The device of any prior embodiment, wherein the distribution of the plurality of particles on the plate is random.

The device of any prior embodiment, wherein the plurality of particles are fixed on the plate and has periodic distribution.

The device of any prior embodiment, wherein the spacer has a flat top.

The device of any prior embodiment, wherein the plurality of particles is temporarily fixed on the first plate, and in an open configuration the sample is deposited first on the first plate before the two plates being bought into the closed configuration.

The device of any prior embodiments, wherein the thickness of the spacer is configured, so that in a closed configuration, for a certain concentration of the analytes in the sample, at least one area of the uniform thickness sample that contains one of the particle becomes optically distinguishable, when viewed outside of the sample layer, from its neighboring area that does not contain a particle.

The device of any prior embodiment, the device comprising two plates and spacers, wherein the pressing is by human hand.

The device of any prior embodiment, the device comprising two plates and spacers, wherein at least a portion of the inner surface of one plate or both plate is hydrophilic.

The device of any prior embodiment, the device comprising two plates and spacers, wherein the inter spacer distance is periodic.

The device of any prior embodiment, the device comprising two plates and spacers, wherein the sample is a deposition directly from a subject to the plate without using any transferring devices.

The device of any prior embodiment, the device comprising two plates and spacers, wherein after the sample deformation at a closed configuration, the sample maintains the same final sample thickness, when some or all of the compressing forces are removed. The device of any prior embodiment, the device comprising two plates and spacers, wherein the spacers have pillar shape and nearly uniform cross-section.

The device of any prior embodiment, the device comprising two plates and spacers, wherein the inter spacer distance (SD) is equal or less than about 120 um (micrometer).

The device of any prior embodiment, the device comprising two plates and spacers, wherein the inter spacer distance (SD) is equal or less than about 100 um (micrometer). The device of any prior embodiment, the device comprising two plates and spacers, wherein the fourth power of the inter-spacer-distance (ISD) divided by the thickness (h) and the Young's modulus (E) of the flexible plate (ISD\hat{ }4/(hE)) is 5×10\hat{ }6 um\hat{ }3/GPa or less. The device of any prior embodiment, the device comprising two plates and spacers, wherein the fourth power of the inter-spacer-distance (ISD) divided by the thickness (h) and the Young's modulus (E) of the flexible plate (ISD\hat{ }4/(hE)) is 5×10\hat{ }5 um3/GPa or less.

The device of any prior embodiment, the device comprising two plates and spacers, wherein the spacers have pillar shape, a substantially flat top surface, a predetermined substantially uniform height, and a predetermined constant inter-spacer distance that is at least about 2 times larger than the size of the analyte, wherein the Young's modulus of the spacers times the filling factor of the spacers is equal or larger than 2 MPa, wherein the filling factor is the ratio of the spacer contact area to the total plate area, and wherein, for each spacer, the ratio of the lateral dimension of the spacer to its height is at least 1 (one).

The device of any prior embodiment, the device comprising two plates and spacers, wherein the spacers have pillar shape, a substantially flat top surface, a predetermined substantially uniform height, and a predetermined constant inter-spacer distance that is at least about 2 times larger than the size of the analyte, wherein the Young's modulus of the spacers times the filling factor of the spacers is equal or larger than 2 MPa, wherein the filling factor is the ratio of the spacer contact area to the total plate area, and wherein, for each spacer, the ratio of the lateral dimension of the spacer to its height is at least 1 (one), wherein the fourth power of the inter-spacer-distance (ISD) divided by the thickness (h) and the Young's modulus (E) of the flexible plate (ISD\hat{ }4/(hE)) is 5×10\hat{ }6 um\hat{ }3/GPa or less.

The device of any prior embodiment, wherein the ratio of the inter-spacing distance of the spacers to the average width of the spacer is 2 or larger, and the filling factor of the spacers multiplied by the Young's modulus of the spacers is 2 MPa or larger.

The method of any prior embodiment, wherein the particle area for the total light signal measurement has substantially the same area as the particle diameter.

The method of any prior embodiment, wherein the particle area for the total light signal measurement is smaller than the area defined by the particle diameter.

The method of any prior embodiment, wherein the analyzing the analyte in the uniform sample layer comprising averaging of the total light signal from each area.

The method of any prior embodiment, wherein the analyzing the analyte in the uniform sample layer comprising (i) taking a ration of the total light signal of each particle area to that of its surrounding area, and (ii) averaging the ratio of all particle area and surround area pairs.

The method of any prior embodiment, wherein the time from the end of the sample deposition to the end of reach a closed configuration is less than 15 seconds.

The method of any prior embodiment, wherein the time from the end of the sample deposition to the end of reach a closed configuration is less than 5 seconds.

The method of any prior embodiment, wherein the surrounding area is 2 D within the edge of the particle.

The method of any prior embodiment, wherein the surrounding area is 5 D within the edge of the particle.

The method of any prior embodiment, wherein the surrounding area is 10 D within the edge of the particle.

The method of any prior embodiment, wherein the surrounding area is 20 D within the edge of the particle.

The method of any prior embodiment, wherein the surrounding area is 50 D within the edge of the particle.

### 1.1 One Step Assay.

In order to achieve one-step assay that detects an analyte in a sample, a key approach of the present invention is to make the captured analyte “visible” in the sample (i.e. that is distinguishable from the rest of the sample) without any washing. The term “captured analyte” refers to the analyte that is being selectively (i.e. specifically) captured by a capture agent.

A captured analyte can give a signal by (a) being attached to a label that can give a signal, (b) giving a signal on its own, and (c) both (a) and (b). Here we focus on the situation (a), wherein the signal from a captured analyte comes from a light label (“label”), wherein the label is capable of selectively attaching to the analyte using a detection agent, and wherein the detection agent can selectively bind to the analyte. However, the invention equally applies to the situations of (b) and (c).

In a one-step assay for situation (a), the objective is to identify/detect the bound labels that are bound to the analyte (the label is termed “bound label”, and the analyte is termed “labeled analyte”) from the labels that are not bound to the analyte (“unbound label”).

In a one-step assay for situation (b), the objective is to identify/detect the bound analyte (i.e. captured by a capture agent) from the analytes that are not captured by a capture agent (“unbound analyte”). When the principle of situation (a) is used to situation (b), the bond label and unbound label in situation (a) becomes the bound analyte and the unbound analyte in the situation (b).

According to the present invention, the one step assay uses two plates to sandwich a thin layer of a sample that has an analyte between the plates, uses a detector above the sample layer to detect a signal from a label, and identify bound label from unbound label through one of the following approaches:
(i) concentrating the bound label into a or a plurality of locations in the sample (termed “concentrated location”), while reduce the concentration of the bound label in the other locations of the sample;
(ii) reducing the local background signal at analyte concentration area (C-LBS), wherein the C-LBS is defined as the background signal generate by the sample volume that is in front of the concentration surface (hence the sample volume is equal to the local sample thickness (from the analyte concentration area to the front plate's inner surface) multiplies by the area of the concentration surface at that location. For example, the C-LBS at a location of a concentration protrusion (with only the protrusion top surface has an analyte concentration area) is the background signal in the sample volume, wherein the volume is equal to the distance between the top of the protrusion to the top plate surface multiplying the area of protrusion's top at the interested location. In this example, clearly the higher the protrusion, the smaller the local background volume, and hence the smaller the C-LBS.
(iii) selectively (i.e. only the bound label, not unbound label) attaching the bound label onto an amplification surface, wherein the amplification surface amplifies the signal of a label only when the label is attached to the surface or within a short distance from the surface (e.g. less than 1 um);
(iv) selectively attaching the bound quencher onto an surface with label, wherein the labeling surface reduces the signal only when the quencher is attached to the surface or within a short distance from the surface (e.g. less than 1 um);
(v) a combination of thereof.

A. Concentrating the Labeled Analyte/Bound Label

Example of embodiments of the present invention for concentrating the labeled analyte/bound label are given below.

(1) Concentration surface. A device for concentrating bound label, comprises: two plates (or an enclosed channel) with a sample (that has an analyte) sandwiched between the two plates, wherein one or both of the plates has a analyte concentration area on its inner surface of the plate, wherein the analyte concentration area has an capture agent that selectively binds the bound label directly or indirectly (i.e. the analyte concentration area has a higher affinity to bind the bound label than the rest area of the plate). An indirect binding means that the capture agent captures an analyte, while the analyte is bound to a label (this is most common case).

The term “analyte concentration area” refers to an area of a surface where the area has a higher affinity to bind the labeled analyte/bound label (or to bind an analyte what later binds a label) than the rest area of the surface.

In some embodiments, a concentration surface can be formed by immobilizing capture agent on the concentration surface, wherein the capture agent specifically bind the analyte.

In some embodiments, a concentration surface can be formed by reducing the binding of the analytes in the surfaces other than the concentration surface.

(2) Concentration protrusion (e.g. pillar). A device for concentrating bound label, comprises: two plates (or an enclosed channel) with a sample (that has an analyte) sandwiched between the two plates, wherein one or both of the plates has a or a plurality of protrusions, wherein the protrusion has a analyte concentration area on at least one of the protrusion's surfaces, wherein the analyte concentration area selectively bind the labeled analyte/bound label.
(3) Concentration bead. A device for concentrating labeled analyte/bound label, comprises: two plates (or an enclosed channel) with a sample (that has an analyte) sandwiched between the two plates, wherein one bead or a plurality of beads is placed in the sample, wherein the bead has a analyte concentration area on the bead's surface, wherein the analyte concentration area selectively bind the bound label.
(4) Combination. Any combination of (1)-(3).

B. Making the Captured Analyte (with Label) Visible

When a detector is used to image an optical signal emitting through the front plate of the sample-plate sandwich, a 2D image will be obtained.

In this 2D image, the requirement for making the analyte concentration area (after catching the labeled analyte) visible (i.e. distinguishable) over the background signal from the latera areas that are not analyte concentration area (i.e. non-analyte concentration area local background signal, “NC-LBS”) is that the signal from the analyte concentration area plus the C-LBL must be larger than NC-LBS by at least one standard variation of the NC-LBS (This condition is termed “visible condition”). The visible condition can be achieved by (i) increase the signal in the analyte concentration area, (ii) reducing C-LBS, (iii) reducing NC-LBS, or (iv) a combination of thereof.

A visible condition can be achieved by adjusting (i) total label concentration in a sample (since some will form bound label with analyte, and the rest will be unbound become a part of background signal), (ii) the total analyte concentration (i.e. limit of detection), (iii) the area or density of the analyte concentration area, (iv) the distance between the analyte concentration area to the front plate, (v) amplification factor of an amplification surface, (vi) the shape of the concentration/amplification area, (vii) the capture reagent concentration on the concentration/amplification area, (viii) the incubation time, or (ix) a combination thereof.

### C. Making Assay Rapid

According to the present invention, an assay can have a short assaying time (i.e. being speeded up) by using the following three approaches: (a) using two plates to sandwich a sample into a thin layer between the plates and by limiting the spacing between the two plates (hence the thickness of at least a port of the sample) into small size (e.g. the spacing is equal to or less than the diffusion parameter (as defined in Definition), since a smaller diffusion parameter will have less diffusion time); (b) making the average lateral distance between two neighboring analyte concentration areas (i.e. inter analyte concentration-area distance (IACD) small (e.g. IACD is equal to or less than 2 times of the diffusion parameter); and (c) (a) and (b).

In certain embodiments, the spacing between the two plate (or the spacer height) is 50 nm, 100 nm, 200 nm, 500 nm, 700 nm, 900 nm, 1 um, 2 um, 3 um, 4 um, 5 um, 6 um, 7 um, 8 um, 9 um, 10 um, 20 um, 30 um, 40 um, 50 um, 60 um, 70 um, 80 um, 90 um, 100 um, 120 um, 150 um, 180 um, 200 um, or in a range between any two of these values.

In some preferred embodiments, the spacing between the two plates (or the spacer height) is 500 nm, 700 nm, 900 nm, 1 um, 2 um, 3 um, 4 um, 5 um, 6 um, 7 um, 8 um, 9 um, 10 um, 20 um, 30 um, 40 um, 50 um, or in a range between any two of these values.

In certain preferred embodiments, the spacing between the two plates (or the spacer height) is 500 nm, 700 nm, 900 nm, 1 um, 2 um, 3 um, 4 um, 5 um, 6 um, 7 um, 8 um, 9 um, 10 um, 20 um, or in a range between any two of these values.

In certain embodiments, the spacing between the two plate (or the spacer height) is 0.01 times of the DP (diffusion parameter), 0.01 times of the DP, 0.1 times of the DP, 0.3 times of the DP, 0.5 times of the DP, 0.7 times of the DP, 1 times of the DP, 1.2 times of the DP, 1.5 times of the DP, 1.8 times of the DP, 2 times of the DP, 2.5 times of the DP, 3 times of the DP, 4 times of the DP, 5 times of the DP, or in a range between any two of these values.

In some preferred embodiments, the spacing between the two plate (or the spacer height) is 0.01 times of the DP (diffusion parameter), 0.05 times of the DP, 0.1 times of the DP, 0.3 times of the DP, 0.5 times of the DP, 0.7 times of the DP, 1 times of the DP, 1.2 times of the DP, 1.5 times of the DP, 1.8 times of the DP, 2 times of the DP, 2.5 times of the DP, or in a range between any two of these values.

In certain preferred embodiments, the spacing between the two plate (or the spacer height) is 0.01 times of the DP (diffusion parameter), 0.05 times of the DP, 0.1 times of the DP, 0.3 times of the DP, 0.5 times of the DP, 0.7 times of the DP, 1 times of the DP, 1.2 times of the DP, 1.5 times of the DP, or in a range between any two of these values.

In certain embodiments, the average IACD is 50 nm, 100 nm, 200 nm, 500 nm, 700 nm, 900 nm, 1 um, 2 um, 3 um, 4 um, 5 um, 6 um, 7 um, 8 um, 9 um, 10 um, 20 um, 30 um, 40 um, 50 um, 60 um, 70 um, 80 um, 90 um, 100 um, 120 um, 150 um, 180 um, 200 um, or in a range between any two of these values.

In some preferred embodiments, the average IACD is 500 nm, 700 nm, 900 nm, 1 um, 2 um, 3 um, 4 um, 5 um, 6 um, 7 um, 8 um, 9 um, 10 um, 20 um, 30 um, 40 um, 50 um, or in a range between any two of these values.

In certain preferred embodiments, the average IACD is 500 nm, 700 nm, 900 nm, 1 um, 2 um, 3 um, 4 um, 5 um, 6 um, 7 um, 8 um, 9 um, 10 um, 20 um, or in a range between any two of these values.

In certain embodiments, the average IACD is 0.01 times of the DP (diffusion parameter), 0.01 times of the DP, 0.1 times of the DP, 0.3 times of the DP, 0.5 times of the DP, 0.7 times of the DP, 1 times of the DP, 1.2 times of the DP, 1.5 times of the DP, 1.8 times of the DP, 2 times of the DP, 3 times of the DP, 4 times of the DP, 5 times of the DP, or in a range between any two of these values.

In some preferred embodiments, the average IACD is 0.01 times of the DP (diffusion parameter), 0.01 times of the DP, 0.1 times of the DP, 0.3 times of the DP, 0.5 times of the DP, 0.7 times of the DP, 1 times of the DP, 1.2 times of the DP, 1.5 times of the DP, 1.8 times of the DP, 2 times of the DP, 2.5 times of the DP, or in a range between any two of these values.

In certain preferred embodiments, the average IACD is 0.01 times of the DP (diffusion parameter), 0.01 times of the DP, 0.1 times of the DP, 0.3 times of the DP, 0.5 times of the DP, 0.7 times of the DP, 1 times of the DP, 1.2 times of the DP, 1.5 times of the DP, or in a range between any two of these values.

In certain preferred embodiments, the average IACD is 500 nm, 700 nm, 900 nm, 1 um, 2 um, 3 um, 4 um, 5 um, 6 um, 7 um, 8 um, 9 um, 10 um, 20 um, or in a range between any two of these values.

In certain embodiments, the intended assay time for the DP is 0.01 sec, 0.1 sec, 0.5 sec, 1 sec, 2 sec, 5 sec, 10 sec, 15 sec, 20 sec, 25 sec, 30 sec, 40 sec, 50 sec, 60 sec, 70 sec, 80 sec, 100 sec, 120 sec, 140 sec, 160 sec, 180 sec, 200 sec, 220 sec, 240 sec, or in a range between any two of these values.

In some preferred embodiments, the intended assay time for the DP is 0.01 sec, 0.1 sec, 0.5 sec, 1 sec, 2 sec, 5 sec, 10 sec, 15 sec, 20 sec, 25 sec, 30 sec, 40 sec, 50 sec, 60 sec, 70 sec, 80 sec, 100 sec, 120 sec, 140 sec, 160 sec, 180 sec, or in a range between any two of these values.

In certain preferred embodiments, the intended assay time for the DP is 0.01 sec, 0.1 sec, 0.5 sec, 1 sec, 2 sec, 5 sec, 10 sec, 15 sec, 20 sec, 25 sec, 30 sec, 40 sec, 50 sec, 60 sec, 70 sec, 80 sec, 100 sec, 120 sec, or in a range between any two of these values.

In certain preferred embodiments, the intended assay time for the DP is 0.01 sec, 0.1 sec, 0.5 sec, 1 sec, 2 sec, 5 sec, 10 sec, 15 sec, 20 sec, 25 sec, 30 sec, 40 sec, 50 sec, 60 sec, or in a range between any two of these values.

In certain embodiments, each of the embodiments has an average IACD and a spacing between the two plate (or a spacer height) that are chosen from the size value or range given in previous paragraphs.

The spacing between the plates can be formed either without using a spacer or with spacers. In some embodiments, the two plates with spacers are parts of a QMAX device (or QMAX card, CROF device, CROF card, which all refer to the same device).

D. Control Plate Spacing and Sample Thickness Using Spacers

According to the present invention, the spacing between the two plates and hence the sample thickness are controlled by using the spacers.

The present invention uses a combination of A to D to achieve a one-step assay.

Spacer height. In some embodiments, all spacers have the same pre-determined height. In some embodiments, spacers have different pre-determined heights. In some embodiments, spacers can be divided into groups or regions, wherein each group or region has its own spacer height. And in certain embodiments, the predetermined height of the spacers is an average height of the spacers. In some embodiments, the spacers have approximately the same height. In some embodiments, a percentage of number of the spacers have the same height.

The height of the spacers is selected by a desired regulated spacing between the plates and/or a regulated final sample thickness and the residue sample thickness. The spacer height (the predetermined spacer height), the spacing between the plates, and/or sample thickness is 3 nm or less, 10 nm or less, 50 nm or less, 100 nm or less, 200 nm or less, 500 nm or less, 800 nm or less, 1000 nm or less, 1 μm or less, 2 μm or less, 3 μm or less, 5 μm or less, 10 μm or less, 20 μm or less, 30 μm or less, 50 μm or less, 100 μm or less, 150 μm or less, 200 μm or less, 300 μm or less, 500 μm or less, 800 μm or less, 1 mm or less, 2 mm or less, 4 mm or less, or in a range between any two of the values.

The spacer height, the spacing between the plates, and/or sample thickness is between 1 nm to 100 nm in one preferred embodiment, 100 nm to 500 nm in another preferred embodiment, 500 nm to 1000 nm in a separate preferred embodiment, 1 μm (i.e. 1000 nm) to 2 μm in another preferred embodiment, 2 μm to 3 μm in a separate preferred embodiment, 3 μm to 5 μm in another preferred embodiment, 5 μm to 10 μm in a separate preferred embodiment, and 10 μm to 50 μm in another preferred embodiment, 50 μm to 100 μm in a separate preferred embodiment.

In some embodiments, the spacer height is controlled precisely. The relative precision of the spacer (i.e. the ratio of the deviation to the desired spacer height) is 0.001% or less, 0.01% or less, 0.1% or less; 0.5% or less, 1% or less, 2% or less, 5% or less, 8% or less, 10% or less, 15% or less, 20% or less, 30% or less, 40% or less, or in a range between any of the values.

In some embodiments, the spacer height, the spacing between the plates, and/or sample thickness is: (i) equal to or slightly larger than the minimum dimension of an analyte, or (ii) equal to or slightly larger than the maximum dimension of an analyte. The “slightly larger” means that it is about 1% to 5% larger and any number between the two values.

In some embodiments, the spacer height, the spacing between the plates, and/or sample thickness is larger than the minimum dimension of an analyte (e.g. an analyte has an anisotropic shape), but less than the maximum dimension of the analyte.

For example, the red blood cell has a disk shape with a minim dimension of 2 μm (disk thickness) and a maximum dimension of 11 μm (a disk diameter). In an embodiment of the present invention, the spacers are selected to make the inner surface spacing of the plates in a relevant area to be 2 μm (equal to the minimum dimension) in one embodiment, 2.2 μm in another embodiment, or 3 (50% larger than the minimum dimension) in other embodiment, but less than the maximum dimension of the red blood cell. Such embodiment has certain advantages in blood cell counting. In one embodiment, for red blood cell counting, by making the inner surface spacing at 2 or 3 μm and any number between the two values, an undiluted whole blood sample is confined in the spacing; on average, each red blood cell (RBC) does not overlap with others, allowing an accurate counting of the red blood cells visually. (Too many overlaps between the RBC's can cause serious errors in counting).

In some embodiments, the spacer height, the spacing between the plates, and/or sample thickness is: (i) equal to or smaller than the minimum dimension of an analyte, or (ii) equal to or slightly smaller than the maximum dimension of an analyte. The “slightly smaller” means that it is about 1% to 5% smaller and any number between the two values.

In some embodiments, the spacer height, the spacing between the plates, and/or sample thickness is larger than the minimum dimension of an analyte (e.g. an analyte has an anisotropic shape), but less than the maximum dimension of the analyte.

In the present invention, in some embodiments, the plates and the spacers are used to regulate not only the thickness of a sample, but also the orientation and/or surface density of the analytes/entity in the sample when the plates are at the closed configuration. When the plates are at a closed configuration, a thinner thickness of the sample results in less analytes/entity per surface area (i.e. less surface concentration).

Spacer lateral dimension. For an open-spacer, the lateral dimensions can be characterized by its lateral dimension (sometimes called width) in the x and y—two orthogonal directions. The lateral dimension of a spacer in each direction is the same or different. In some embodiments, the lateral dimension for each direction (x or y) is 1 nm or less, 3 nm or less, 5 nm or less, 7 nm or less, 10 nm or less, 20 nm or less, 30 nm or less, 40 nm or less, 50 nm or less, 100 nm or less, 200 nm or less, 500 nm or less, 800 nm or less, 1000 nm or less, 1 μm or less, 2 μm or less, 3 μm or less, 5 μm or less, 10 μm or less, 20 μm or less, 30 μm or less, 50 μm or less, 100 μm or less, 150 μm or less, 200 μm or less, 300 μm or less, or 500 μm or less, or in a range between any two of the values.

In some embodiments, the ratio of the lateral dimensions of x to y direction is 1, 1.5, 2, 5, 10, 100, 500, 1000, 10,000, or in a range between any two of the value. In some embodiments, a different ratio is used to regulate the sample flow direction; the larger the ratio, the flow is along one direction (larger size direction).

In some embodiments, different lateral dimensions of the spacers in x and y direction are used as (a) using the spacers as scale-markers to indicate the orientation of the plates, (b) using the spacers to create more sample flow in a preferred direction, or both.

In a preferred embodiment, the period, width, and height of the spacers are substantially the same. In some embodiments, all spacers have the same shape and dimensions. In some embodiments, the spacers have different lateral dimensions.

For enclosed-spacers, in some embodiments, the inner lateral shape and size are selected based on the total volume of a sample to be enclosed by the enclosed spacer(s), wherein the volume size has been described in the present disclosure; and in certain embodiments, the outer lateral shape and size are selected based on the needed strength to support the pressure of the liquid against the spacer and the compress pressure that presses the plates.

In certain embodiments, the aspect ratio of the height to the average lateral dimension of the pillar spacer is 100,000, 10,000, 1,000, 100, 10, 1, 0.1, 0.01, 0.001, 0.0001, 0, 00001, or in a range between any two of the values.

Inter-spacer distance. The spacers can be a single spacer or a plurality of spacers on the plate or in a relevant area of the sample. In some embodiments, the spacers on the plates are configured and/or arranged in an array form, and the array is a periodic, non-periodic array or periodic in some locations of the plate while non-periodic in other locations.

In some embodiments, the periodic array of the spacers is arranged as lattices of square, rectangle, triangle, hexagon, polygon, or any combinations of thereof, where a combination means that different locations of a plate has different spacer lattices.

In some embodiments, the inter-spacer distance of a spacer array is periodic (i.e. uniform inter-spacer distance) in at least one direction of the array. In some embodiments, the inter-spacer distance is configured to improve the uniformity between the plate spacing at a closed configuration.

In some embodiments, the distance between neighboring spacers (i.e. the inter-spacer distance) is 1 μm or less, 5 μm or less, 7 μm or less, 10 μm or less, 20 μm or less, 30 μm or less, 40 μm or less, 50 μm or less, 60 μm or less, 70 μm or less, 80 μm or less, 90 μm or less, 100 μm or less, 200 μm or less, 300 μm or less, 400 μm or less, or in a range between any two of the values.

In certain embodiments, the inter-spacer distance is at 400 μm or less, 500 μm or less, 1 mm or less, 2 mm or less, 3 mm or less, 5 mm or less, 7 mm or less, 10 mm or less, or in any range between the values. In certain embodiments, the inter-spacer distance is a10 mm or less, 20 mm or less, 30 mm or less, 50 mm or less, 70 mm or less, 100 mm or less, or in any range between the values.

The distance between neighboring spacers (i.e. the inter-spacer distance) is selected so that for a given properties of the plates and a sample, at the closed-configuration of the plates, the sample thickness variation between two neighboring spacers is, in some embodiments, at most 0.5%, 1%, 5%, 10%, 20%, 30%, 50%, 80%, or in any range between the values; or in certain embodiments, at most 80%, 100%, 200%, 400%, or in a range between any two of the values.

Clearly, for maintaining a given sample thickness variation between two neighboring spacers, when a more flexible plate is used, a closer inter-spacer distance is needed.

In a preferred embodiment, the spacer is a periodic square array, wherein the spacer is a pillar that has a height of 2 to 4 μm, an average lateral dimension of from 1 to 20 μm, and inter-spacer spacing of 1 μm to 100 μm.

In a preferred embodiment, the spacer is a periodic square array, wherein the spacer is a pillar that has a height of 2 to 4 μm, an average lateral dimension of from 1 to 20 μm, and inter-spacer spacing of 100 μm to 250 μm.

In a preferred embodiment, the spacer is a periodic square array, wherein the spacer is a pillar that has a height of 4 to 50 μm, an average lateral dimension of from 1 to 20 μm, and inter-spacer spacing of 1 μm to 100 μm.

In a preferred embodiment, the spacer is a periodic square array, wherein the spacer is a pillar that has a height of 4 to 50 μm, an average lateral dimension of from 1 to 20 μm, and inter-spacer spacing of 100 μm to 250 μm.

The period of spacer array is between 1 nm to 100 nm in one preferred embodiment, 100 nm to 500 nm in another preferred embodiment, 500 nm to 1000 nm in a separate preferred embodiment, 1 μm (i.e. 1000 nm) to 2 μm in another preferred embodiment, 2 μm to 3 μm in a separate preferred embodiment, 3 μm to 5 μm in another preferred embodiment, 5 μm to 10 μm in a separate preferred embodiment, and 10 μm to 50 μm in another preferred embodiment, 50 μm to 100 μm in a separate preferred embodiment, 100 μm to 175 μm in a separate preferred embodiment, and 175 μm to 300 μm in a separate preferred embodiment.

Spacer density. The spacers are arranged on the respective plates at a surface density of greater than one per ρm², greater than one per 10 μm², greater than one per 100 μm², greater than one per 500 μm², greater than one per 1000 μm², greater than one per 5000 μm², greater than one per 0.01 mm², greater than one per 0.1 mm², greater than one per 1 mm², greater than one per 5 mm², greater than one per 10 mm², greater than one per 100 mm², greater than one per 1000 mm², greater than one per 10000 mm², or in a range between any two of the values. In some embodiments, the spacers have a density of at least 1/mm², at least 10/mm², at least 50/mm², at least 100/mm², at least 1,000/mm², or at least 10,000/mm².

Spacer area filling factor is defined as the ratio of spacer area to the total area or the ratio of spacer period to the width. In some embodiments, the filling factor is at least 1%, 2%, 3%, 4%, 5%, 6%, 7%, 8%, 9%, 10%, 20%, or in the range between any of the two values. In certain embodiments, the filling factor is at least 2.3%.

The device that comprises two plates and spacers, wherein the fourth power of the inter-spacer-distance (ISD) divided by the thickness (h) and the Young's modulus (E) of the flexible plate (ISD\hat{ }4/(hE)) is 5×10\hat{ }6 um\hat{ }3/GPa or less.

The device that comprises two plates and spacers, wherein the fourth power of the inter-spacer-distance (ISD) divided by the thickness (h) and the Young's modulus (E) of the flexible plate (ISD\hat{ }4/(hE)) is 5×10\hat{ }5 um3/GPa or less.

The device that comprises two plates and spacers, wherein the spacers have pillar shape, a substantially flat top surface, a predetermined substantially uniform height, and a predetermined constant inter-spacer distance that is at least about 2 times larger than the size of the analyte, wherein the Young's modulus of the spacers times the filling factor of the spacers is equal or larger than 2 MPa, wherein the filling factor is the ratio of the spacer contact area to the total plate area, and wherein, for each spacer, the ratio of the lateral dimension of the spacer to its height is at least 1 (one).

The device that comprises two plates and spacers, wherein the spacers have pillar shape, a substantially flat top surface, a predetermined substantially uniform height, and a predetermined constant inter-spacer distance that is at least about 2 times larger than the size of the analyte, wherein the Young's modulus of the spacers times the filling factor of the spacers is equal or larger than 2 MPa, wherein the filling factor is the ratio of the spacer contact area to the total plate area, and wherein, for each spacer, the ratio of the lateral dimension of the spacer to its height is at least 1 (one), wherein the fourth power of the inter-spacer-distance (ISD) divided by the thickness (h) and the Young's modulus (E) of the flexible plate (ISD\hat{ }4/(hE)) is 5×10\hat{ }6 um\hat{ }3/GPa or less.

The device that comprises two plates and spacers, wherein the ratio of the inter-spacing distance of the spacers to the average width of the spacer is 2 or larger, and the filling factor of the spacers multiplied by the Young's modulus of the spacers is 2 MPa or larger.

2. Multiplexed Assays

It is another aspect of the present invention to provide devices and methods with multiplexing capability for homogeneous assays.

In some embodiments, the sample comprises more than one analyte of interest, and there is need to detect the more than one analytes simultaneously using the same device (“multiplexing”).

In some embodiments, the device for multiplexed homogeneous assays comprises: a first plate, a second plate, and spacers. In some embodiments, the plates are movable relative to each other into different configurations, including an open configuration and a closed configuration. In some embodiments, each of the plates has, on its respective inner surface, a sample contact area for contacting a sample suspected of containing a first analyte and a second analyte. In some embodiments, one or both of the plates comprise the spacers, at least one of the spacers is inside the sample contact area, and the spacers have a predetermined substantially uniform height. In some embodiments, one or both of the plates comprise, on the respective inner surface, a plurality of first beads and second beads, wherein the first and second beads have first and second capture agents immobilized thereon, respectively. In some embodiments, the first and second capture agents are capable of binding to and immobilizing the first and second analytes, respectively.

In some embodiments, in the open configuration of the device for multiplexed assays, the two plates are partially or entirely separated apart, the spacing between the plates is not regulated by the spacers, and the sample is deposited on one or both of the plates.

In some embodiments, in the closed configuration of the device for multiplexed assays, at least part of the sample is compressed by the two plates into a layer of highly uniform thickness, the uniform thickness of the layer is confined by the inner surfaces of the plates and is regulated by the plates and the spacers, the analytes in the layer of uniform thickness are concentrated by the beads so that signal of the captured analytes on the beads is distinguishable from signal emanating from other area in the layer of uniform thickness.

In some embodiments, the assay is designed to detect analytes of two different species. In some embodiments, the number of analyte species the assay is designed to detect is 3, 4, 5, 6, 7, 8, 10 or more, 20 or more, 30 or more, 100 or more, or an integral number in a range between any two of these values.

In multiplexed assays, it is often critical to distinguish the signals from different assays. In some embodiments of the present invention, the signals of the captured first and second analytes are distinguishable from one another by one of the following designs or methods:

(1) different types of labels are attached to the analytes of different species directly or the different detection agents that bind to the analytes of corresponding species;

(2) different types of beads are used to capture analytes of different species, and the bead types are distinguishable by the detection methods; and

(3) a combination of (1) and (2).

In some embodiments, the beads for different analytes (e.g. the first and second beads) are different in their sizes.

In some embodiments, the beads for different analytes (e.g. the first and second beads) are different in their optical properties selected from the group consisting of: photoluminescence, electroluminescence, and electrochemiluminescence, light absorption, reflection, transmission, diffraction, scattering, diffusion, surface Raman scattering, and any combination thereof.

In some embodiments, the beads for different analytes (e.g. the first and second beads) are different in their electric densities, and a detector that can detect electric density is used.

The three different exemplary processes of multiplexed homogeneous assays are described below.

the first embodiment the case where beads of different colors are used to capture analytes of different species (symbolized by the different shapes in the sample). In this case, a detector with the capability of visualizing or imaging the sample under bright-field illumination is used to facilitate the virtual separation of signals from analytes of different species. For instance, in some embodiments, the bright-field images are superimposed with the fluorescent images to sort out the signals, when the assay signals (signal of the analytes or the bound detection agents) are fluorescent.

The second embodiment the case where beads of different sizes are used to capture analytes of different species (symbolized by the different shapes in the sample). In this case, a detector with the capability of detecting the geometric distribution of the signal of the capture analytes or visualizing or imaging the beads under bright-field illumination is used to facilitate the virtual separation of signals from analytes of different species. For instance, in some embodiments, the assay signals are fluorescent, a detector that can image the fluorescent signals is able to record the geometric distribution of the fluorescent signal on the surface of the beads. A skilled artisan can separate beads of different sizes based on the fluorescent images. In other cases, bright-field images of the beads are used to aid the separation of the signals.

The third embodiment shows the case where different labels are used to separate analyte of different species (symbolized by the different shapes in the sample). In this exemplary case, different fluorophores are attached to the detection agents that bind to analytes of different species. A detector that can image the sample under fluorescent mode and is equipped with emission filters with different wavelengths of light should be used to distinguish the signals of different analytes.

The three different exemplary processes of multiplexed homogeneous nucleic acid hybridization assays are given below

The first example is the case where beads of different colors are used to capture analytes of different species (symbolized by the different colors in the sample). In this case, a detector with the capability of visualizing or imaging the sample under bright-field illumination is used to facilitate the virtual separation of signals from analytes of different species. For instance, in some embodiments, the bright-field images are superimposed with the fluorescent images to sort out the signals, when the assay signals (signal of the analytes or the bound detection agents) are fluorescent.

The fisecond example is the case where beads of different sizes are used to capture analytes of different species (symbolized by the different colors in the sample). In this case, a detector with the capability of detecting the geometric distribution of the signal of the capture analytes or visualizing or imaging the beads under bright-field illumination is used to facilitate the virtual separation of signals from analytes of different species. For instance, in some embodiments, the assay signals are fluorescent, a detector that can image the fluorescent signals is able to record the geometric distribution of the fluorescent signal on the surface of the beads. A skilled artisan can separate beads of different sizes based on the fluorescent images. In other cases, bright-field images of the beads are used to aid the separation of the signals.

The first example is the case where different labels are used to separate analyte of different species (symbolized by the different color in the sample). In this exemplary case, different fluorophores are attached to the detection agents that bind to analytes of different species. A detector that can image the sample under fluorescent mode and is equipped with emission filters with different wavelengths of light should be used to distinguish the signals of different analytes.

3. Assays, Capture Agent, and Detection Agent

In some embodiments, the assay is a sandwich assay, in which capture agent and detection agent are configured to bind to analyte at different locations thereof, forming capture agent-analyte-detection agent sandwich.

In some embodiments, the assay is a competitive assay, in which analyte and detection agent compete with each other to bind to the capture agent.

In some embodiments, the assay is an immunoassay, in which protein analyte is detected by antibody-antigen interaction. In some embodiments, the assay is a nucleic acid assay, in which nucleic acids (e.g. DNA or RNA) are detected by hybridization with complementary oligonucleotide probes.

In some embodiments, the assay utilizes light signals as readout. In some embodiments, the assay utilizes magnetic signals as readout. In some embodiments, the assay utilizes electric signals as readout. In some embodiments, the assay utilizes signals in any other form as readout.

In some embodiments, the light signal from the assay is luminescence selected from photoluminescence, electroluminescence, and electrochemiluminescence. In some embodiments, the light signal is light absorption, reflection, transmission, diffraction, scattering, or diffusion. In some embodiments, the light signal is surface Raman scattering. In some embodiments, the electrical signal is electrical impedance selected from resistance, capacitance, and inductance. In some embodiments, the magnetic signal is magnetic relaxivity. In some embodiments, the signal is any combination of the foregoing signal forms.

The capture antibodies capture the protein analyte in a sample, which is further bound with labeled detection antibodies. In this case, the capture antibody and detection antibody are configured to bind to the protein analyte at its different locations, therefore forming a capture antibody-protein analyte-detection antibody sandwich. Panel (B) shows a nucleic acid concentration surface, which is coated with oligonucleotide capture probes. The capture probes are complementary to one part of the nucleic acid analyte, therefore capturing the analyte to the surface. Further, the analyte is bound with a labeled detection probe that is complementary to another part of the analyte. Panel (C) shows another case of protein concentration surface, where protein analyte is directly labeled by an optical label and captured by the capture antibodies that are coated on the concentration surface. Panel (D) shows another case of protein concentration surface, where protein analyte is bound with a quencher, which quenches the signal emitted by the label that is associated with the capture antibodies on the concentration surface. In this case, the concentration of the protein analyte to the concentration surface reduces the signal emanating from the concentration surface.

In some embodiments, the capture agent and the detection agent are configured to bind to the analyte at different locations thereof and to form a capture agent-analyte-detection agent sandwich that is immobilized to the separated nano-/micro-islands on one or both of the plates; wherein the shape of nano- or micro-islands are selected from the group consisting of sphere, rectangle, hexagon, and/or any other polyhedron, with lattice of square, hexagon, and/or any other lattices. The separated nano/micro islands are on one or both of the plates with (i) round shape with square lattice (ii) rectangle shape with square lattice (iii) triangle shape with hexagonal lattice (iv) round shape with aperiodicity.

In some embodiments, the material of protrusions that are nano or micro islands are selected from the group consisting of plastic as polystyrene, polypropylene, polycarbonate, PMMA, PET; metals as gold, aluminum, silver, copper, tin and/or their combinations; or any other material whose surface can be modified to be associated with the capture agent.

As discussed above, in some embodiments, the beads, the capture agent, and the detection agent are configured to render signal of the bead-captured analyte distinguishable from signal of free detection agent in the layer of uniform thickness. In some embodiments, it is critical to achieve the foregoing configuration, in that only if the signal from the sandwich structure is distinguishable from the “background” signal of the free detection agent in the layer of uniform thickness, one can use the detected signals as a readout of the presence and/or quantity of the analyte in the sample, thereby realizing the assay.

In some embodiments, the target analyte competes with the detection agent on the capture locations on beads. When more target analyte appears, beads become relative dark.

In some embodiments, the beads are associated with a label, and the detection agent is a quencher that is configured to quench signal of the beads-associated label when the detection agent is in proximity of the label. When beads capture the target analyte, the label on beads become quenched or dimed.

In some embodiments, the capture agent includes, but not limited to, protein, peptide, peptidomimetics, streptavidin, biotin, oligonucleotide, oligonucleotide mimetics, any other affinity ligand and any combination thereof. In some embodiments, the capture agent is an antibody. In some embodiments, the capture antibody is an anti-C Reactive Protein (CRP) antibody.

In some embodiments, the capture agent has a concentration that is sufficient to detect the presence and/or measure the amount of the analyte. In some embodiments, the capture agent has a concentration that is sufficient to immobilize the analyte.

In some embodiments, the detection agent includes, but not limited to, protein, peptide, peptidomimetics, streptavidin, biotin, oligonucleotide, oligonucleotide mimetics, any other affinity ligand and any combination thereof. In some embodiments, the detection agent is an antibody. In some embodiments, the detection antibody is an anti-CRP antibody.

In some embodiments, the detection antibody is configured to have a concentration in the layer of uniform thickness that is higher than analyte concentration in the sample. In some embodiments, the ratio of the detection antibody concentration over the analyte concentration is 1 or more, 2 or more, 5 or more, 10 or more, 20 or more, 30 or more, 50 or more, 100 or more, 200 or more, 300 or more, 500 or more, 1000 or more, or in a range between any two of these values.

In some embodiments, the detection antibody is labeled. In some embodiments, the label can be fluorescent, colorimetric or luminescent. In some embodiments, the detection antibody is labeled with a fluorophore. In some embodiments, the fluorophores include, but are not limited to, IRDye800CW, Alexa 790, Dylight 800, fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1 (5,5′,6,6′-tetrachloro-1,1′,3,3′-tetraethylbenzimidazoylcarbocyanine iodide), tetrabromorhodamine 123, rhodamine 6G, TMRM (tetramethyl rhodamine methyl ester), TMRE (tetramethyl rhodamine ethyl ester), tetramethylrosamine, rhodamine B and 4-di methylaminotetramethylrosamine, green fluorescent protein, blue-shifted green fluorescent protein, cyan-shifted green fluorescent protein, red-shifted green fluorescent protein, yellow-shifted green fluorescent protein, 4-acetamido-4′-isothiocyanatostilbene-2,2′disulfonic acid; acridine and derivatives, such as acridine, acridine isothiocyanate; 5-(2′-aminoethyl)aminonaphthalene-1-sulfonic acid (EDANS); 4-amino-N-[3-vinylsulfonyl)phenyl]naphth-alimide-3,5 disulfonate; N-(4-anilino-1-naphthyl)maleimide; anthranilamide; 4,4-difluoro-5-(2-thienyl)-4-bora-3a,4a diaza-5-indacene-3-propioni-c acid BODIPY; cascade blue; Brilliant Yellow; coumarin and derivatives: coumarin, 7-amino-4-methylcoumarin (AMC, Coumarin 120), 7-amino-4-trifluoromethylcoumarin (Coumarin 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2-,2′-disulfonic acid; 4,4′-diisothiocyanatostilbene-2,2′-disulfonic acid; 5-(dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM), 5-(4,6-dichlorotriazin-2-yl)amino--fluorescein (DTAF), 2′,7′dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelli-feroneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl hodamine isothiocyanate (TRITC); riboflavin; 5-(2′-aminoethyl) aminonaphthalene-1-sulfonic acid (EDANS), 4-(4′-dimethylaminophenylazo)benzoic acid (DABCYL), rosolic acid; CAL Fluor Orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, fluorescent europium and terbium complexes; combinations thereof, and the like. Suitable fluorescent proteins and chromogenic proteins include, but are not limited to, a green fluorescent protein (GFP), including, but not limited to, a GFP derived from Aequoria victoria or a derivative thereof, e.g., a “humanized” derivative such as Enhanced GFP; a GFP from another species such as Renilla reniformis, Renilla mulleri, or Ptilosarcus guernyi; “humanized” recombinant GFP (hrGFP); any of a variety of fluorescent and colored proteins from Anthozoan species; combinations thereof; and the like.

In some embodiments, the beads are treated with a protein stabilizer. In some embodiments, the beads can be deposited on the plate and dried (e.g. air-dried), further simplifying the process. In some embodiments, the detection antibody is placed on one of the plates and dried. In some embodiments, the plate with the detection antibody is treated with protein stabilizer. In some embodiments, the detection antibody with protein stabilizer is pre-printed on one of the plates and air dried.

In some embodiments, wherein the beads are prepared by:

(a) activating with N-Hydroxysuccinimide (NHS);

(b) blocking with a BSA solution; and

(c) incubating with a capture agent solution.

4. Detector, System and Smartphone-Based System

Another aspect of the present invention provides a system for homogeneous assay. In some embodiments, the system comprises the device as discussed above and a detector that detects the analyte in the layer of uniform thickness.

In some embodiments, detector detects a signal from the capture agent-analyte-detection agent sandwich indicative of the presence and/or quantity of the analyte.

In some embodiments, the signal is:
i. luminescence selected from photoluminescence, electroluminescence, and electrochemiluminescence;
ii. light absorption, reflection, transmission, diffraction, scattering, or diffusion;
iii. surface Raman scattering;
iv. electrical impedance selected from resistance, capacitance, and inductance;
v. magnetic relaxivity; or
vi. any combination of i-v.

Another aspect of the present invention provides a smartphone system for homogeneous assay. In some embodiments, the smartphone system comprises:
(a) a device of any aforementioned embodiment;
(b) a mobile communication device that comprises:
i. one or a plurality of cameras for detecting and/or imaging the sample;
ii. electronics, signal processors, hardware and software for receiving and/or processing the detected signal and/or the image of the sample and for remote communication; and
(c) an adaptor configured to hold the closed device and engageable to mobile communication device;

wherein when engaged with the mobile communication device, the adaptor is configured to facilitate the detection and/or imaging of the analyte in the sample at the closed configuration.

In some embodiments, the mobile communication device is configured to communicate test results to a medical professional, a medical facility or an insurance company.

In some embodiments, the mobile communication device is further configured to communicate information on the subject with the medical professional, medical facility or insurance company.

In some embodiments, the mobile communication device is configured to receive a prescription, diagnosis or a recommendation from a medical professional.

In some embodiments, the mobile communication device communicates with the remote location via a wifi or cellular network.

In some embodiments, the mobile communication device is a mobile phone.

In some embodiments, the images can be taken by a camera that is part of a mobile device. In some embodiments, the mobile device is a smart phone.

In the local reading approach, as shown in FIG. 1B, one or more than one particles will be measured for the following two measurements: (a) the signal from the particle region (S_{P}). It can be from the whole particle region or a designated area of the particle region; and (b) the signal of area around the particle (local background S_{B}). It can be from the whole area around the particle or a designated area. The definition of “around” can be a distance of 0.01D, 0.1D, 0.2D, 0.5D, 1D, 2D, 5D, 10D, 50D or a range between any two of the values to the outer surface of the particle, in which “D” is the average diameter of the particle. The true Signal of Assay (S_{A}) for each particle can be determined as S_{A}=S_{P}−S_{B}. The assay signal from each CROF (S_{CROF}) can be the average of multiple particles. It can be all particles on a whole CROF or particles in a designated region of a CROF (e.g., S_{CROF}=Average (S_{A1}, S_{A2}, S_{A3} . . . S_{An}))

5. Analyte, Sample and Application

In some embodiments, the analyte to be detected in the homogeneous assay includes, but not limited to, cells, viruses, proteins, peptides, DNAs, RNAs, oligonucleotides, and any combination thereof.

In some embodiments, the present invention finds use in detecting biomarkers for a disease or disease state. In certain instances, the present invention finds use in detecting biomarkers for the characterization of cell signaling pathways and intracellular communication for drug discovery and vaccine development. For example, the present invention may be used to detect and/or quantify the amount of biomarkers in diseased, healthy or benign samples. In certain embodiments, the present invention finds use in detecting biomarkers for an infectious disease or disease state. In some cases, the biomarkers can be molecular biomarkers, such as but not limited to proteins, nucleic acids, carbohydrates, small molecules, and the like. The present invention find use in diagnostic assays, such as, but not limited to, the following: detecting and/or quantifying biomarkers, as described above; screening assays, where samples are tested at regular intervals for asymptomatic subjects; prognostic assays, where the presence and or quantity of a biomarker is used to predict a likely disease course; stratification assays, where a subject's response to different drug treatments can be predicted; efficacy assays, where the efficacy of a drug treatment is monitored; and the like.

The present invention has applications in (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals.

In some embodiments, the liquid sample is made from a biological sample selected from the group consisting of: amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, urine, and any combination thereof.

In some embodiments, the sample is an environmental liquid sample from a source selected from the group consisting of: river, lake, pond, ocean, glaciers, icebergs, rain, snow, sewage, reservoirs, tap water, or drinking water, solid samples from soil, compost, sand, rocks, concrete, wood, brick, sewage, and any combination thereof.

In some embodiments, the sample is an environmental gaseous sample from a source selected from the group consisting of: the air, underwater heat vents, industrial exhaust, vehicular exhaust, and any combination thereof.

In some embodiments, the sample is a foodstuff sample selected from the group consisting of: raw ingredients, cooked food, plant and animal sources of food, preprocessed food, and partially or fully processed food, and any combination thereof.

6. Examples of Present Invention

### Multiplexed Assay

NA1. A device for a homogeneous assay, comprising:

a first plate, a second plate, spacers, a plurality of particles, and capture agents, wherein:
vi. the first and second plates are movable relative to each other into different configurations, including an open configuration and a closed configuration;
vii. each of the plates has, on its respective inner surface, a sample contact area for contacting a sample suspected of containing an analyte;
viii. the first plate comprises the spacers that are fixed on its inner surface, at least one of the spacers is inside the sample contact area, the spacers have a predetermined substantially uniform height that is equal to 100 um or less;
ix. the plurality of particles has the capture agents immobilized on their surface, wherein the capture agents are capable of specifically binding and immobilizing the analyte; and
x. the plurality of particles are (a) distributed on the sample contact area of the first plate, except the areas occupied by the spacers, and (b) are temporarily or permanently fixed on the first plate;

wherein in the open configuration, the two plates are partially or entirely separated apart, the spacing between the plates is not regulated by the spacers, and the sample is deposited on one or both of the plates; and

wherein in the closed configuration, which is configured after deposition of the sample in the open configuration: at least part of the sample is compressed by the two plates into a layer of highly uniform thickness, the uniform thickness of the layer is confined by the inner surfaces of the plates and is regulated by the plates and the spacers.

NB1. A device for a homogeneous assay, comprising:

a first plate, a second plate, spacers, a plurality of particles, and capture agents, wherein:
vi. the first and second plates are movable relative to each other into different configurations, including an open configuration and a closed configuration;
vii. each of the plates has, on its respective inner surface, a sample contact area for contacting a sample suspected of containing a analyte;
viii. one or both plates comprises the spacers that are fixed on its inner surface, at least one of the spacers is inside the sample contact area, the spacers have a predetermined substantially uniform height that is equal to 100 um or less;
ix. the plurality of particles has the capture agents immobilized on their surface, wherein the capture agents are capable of specifically binding and immobilizing the analyte; and
x. the plurality of particles are (a) distributed on a sample contact area of the first, and (b) are temporarily or permanently fixed on the plate;

wherein in the open configuration, the two plates are partially or entirely separated apart, the spacing between the plates is not regulated by the spacers, and the sample is deposited on one or both of the plates; and

wherein in the closed configuration, which is configured after deposition of the sample in the open configuration: at least part of the sample is compressed by the two plates into a layer of highly uniform thickness, the uniform thickness of the layer is confined by the inner surfaces of the plates and is regulated by the plates and the spacers.

NC1. The device of any prior embodiment, wherein the distribution of the plurality of particles on the plate is random.
NC2. The device of any prior embodiment, wherein the plurality of particles is fixed on the plate and has periodic distribution.
NC3. The device of any prior embodiment, wherein the spacer has a flat top.
NC4. The device of any prior embodiment, wherein the plurality of particles is temporarily fixed on the first plate, and in an open configuration the sample is deposited on the first plate before the two plates are brought into the closed configuration.
NC5. The device of any prior embodiment, wherein the thickness of the spacer is configured such that, in a closed configuration, for a certain concentration of the analytes in the sample, at least one area of the uniform thickness sample that contains one of the particle becomes optically distinguishable, when viewed outside of the sample layer, from its neighboring area that does not contain a particle.
NC6. The device of any prior embodiment, the device comprising two plates and spacers, wherein the pressing is by human hand.
NC7. The device of any prior embodiment, wherein the diameter of one or more of the plurality of particles is equal to the height of the spacers.
NC8. The device of any prior embodiment, wherein the spacer height is about 10 um.
NC9. The device of any prior embodiment, wherein the spacer height is about 5 um.
NC10. The device of any prior embodiment, wherein the spacer height is between about 0.1 um and about 15 um.
NC11. The device of any prior embodiment, wherein the spacer height is between about 0.1 um and about 3 um.
NC12. The device of any prior embodiment, wherein at least a portion of the inner surface of one plate or both plate is hydrophilic.
NC13. The device of any prior embodiment, wherein the inter spacer distance is periodic.
NC14. The device of any prior embodiment, wherein the sample is a deposition directly from a subject to the plate without using any transferring devices.
NC15. The device of any prior embodiment, wherein after the sample deformation at a closed configuration, the sample maintains the same final sample thickness, when some or all of the compressing forces are removed.
NC16. The device of any prior embodiment, wherein the spacers have pillar shape and nearly uniform cross-section.
NC17. The device of any prior embodiment, wherein the inter spacer distance (SD) is equal or less than about 120 um (micrometer).
NC18. The device of any prior embodiment, wherein the inter spacer distance (SD) is equal or less than about 100 um (micrometer).
NC19. The device of any prior embodiment, wherein the fourth power of the inter-spacer-distance (ISD) divided by the thickness (h) and the Young's modulus (E) of the flexible plate (ISD\hat{ }4/(hE)) is 5×10\hat{ }6 um\hat{ }3/GPa or less.
NC20. The device of any prior embodiment, wherein the fourth power of the inter-spacer-distance (ISD) divided by the thickness (h) and the Young's modulus (E) of the flexible plate (ISD\hat{ }4/(hE)) is 5×10\hat{ }5 um3/GPa or less.
NC21. The device of any prior embodiment, wherein the spacers have pillar shape, a substantially flat top surface, a predetermined substantially uniform height, and a predetermined constant inter-spacer distance that is at least about 2 times larger than the size of the analyte, wherein the Young's modulus of the spacers times the filling factor of the spacers is equal or larger than 2 MPa, wherein the filling factor is the ratio of the spacer contact area to the total plate area, and wherein, for each spacer, the ratio of the lateral dimension of the spacer to its height is at least 1 (one).
NC22. The device of any prior embodiment, wherein the spacers have pillar shape, a substantially flat top surface, a predetermined substantially uniform height, and a predetermined constant inter-spacer distance that is at least about 2 times larger than the size of the analyte, wherein the Young's modulus of the spacers times the filling factor of the spacers is equal or larger than 2 MPa, wherein the filling factor is the ratio of the spacer contact area to the total plate area, and wherein, for each spacer, the ratio of the lateral dimension of the spacer to its height is at least 1 (one), wherein the fourth power of the inter-spacer-distance (ISD) divided by the thickness (h) and the Young's modulus (E) of the flexible plate (ISD\hat{ }4/(hE)) is 5×10\hat{ }6 um\hat{ }3/GPa or less.
NC23. The device of any prior device embodiment, wherein the ratio of the inter-spacing distance of the spacers to the average width of the spacer is 2 or larger, and the filling factor of the spacers multiplied by the Young's modulus of the spacers is 2 MPa or larger.
ND1. A method of performing a homogeneous assay, comprising the steps of:

(a) obtaining a sample suspected of containing an analyte;

(b) obtaining a device of any prior embodiment, wherein the capture agents are capable of specifically binding an binding site of the analyte;

(c) having optical labels on at least a part of the sample contact areas of the device, wherein the optical labels are capable of binding to the analytes;

(d) depositing the sample on one or both of the plates when the plates are in an open configuration, wherein in an open configuration;

(e) after (d), bringing the two plates together and pressing the plates into a closed configuration, at least part of the sample is compressed by the two plates into a layer of highly uniform thickness, the uniform thickness of the layer is confined by the inner surfaces of the plates and is regulated by the plates and the spacers;

(f) while the plates are in the closed configuration, analyzing the analyte in the layer of uniform thickness, wherein the analyzing comprises:

i. measuring, from outside of the sample layer, the total light signal from (a) a particle area that is an area of the sample layer that contains one particle and from (b) a surrounding area that is the area of the sample layer which is around the particle area, wherein the surrounding area is 50 D within the edge of the particle, wherein the D is the diameter of the particle; and

ii. measuring the total light signal from each of the particle area and the surrounding area of at least two different particle areas.

ND2. The method of any prior embodiment, wherein the particle area for the total light signal measurement has substantially the same area as the particle diameter.
ND3. The method of any prior embodiment, wherein the particle area for the total light signal measurement is smaller than the area defined by the particle diameter.
ND4. The method of any prior embodiment, wherein the analyzing the analyte in the uniform sample layer comprises averaging of the total light signal from each area.
ND5. The method of any prior embodiment, wherein the analyzing the analyte in the uniform sample layer comprises (i) taking a ratio of the total light signal of each particle area to that of its surrounding area, and (ii) averaging the ratio of all particle area and surround area pairs.
ND6. The method of any prior embodiment, wherein the time from the end of the sample deposition to the end of the plates being pressed into the closed configuration is less than 15 seconds.
ND7. The method of any prior embodiment, wherein the time from the end of the sample deposition to the end of the plates being pressed into the closed configuration is less than 5 seconds.
NE1. An apparatus for homogeneous assaying an analyte in a sample, comprising:
i. a device of any prior embodiment; and
ii. one or more imagers that image at least a part of the sample contact area.

A device for rapid multiplexed homogeneous assay, comprising:

a first plate, a second plate, and spacers, wherein:
xi. the plates are movable relative to each other into different configurations, including an open configuration and a closed configuration;
xii. each of the plates has, on its respective inner surface, a sample contact area for contacting a sample suspected of containing a first analyte and a second analyte;
xiii. one or both of the plates comprise the spacers, at least one of the spacers is inside the sample contact area, and the spacers have a predetermined substantially uniform height;
xiv. one or both of the plates comprise, on the respective inner surface, a plurality of first beads and second beads, wherein the first and second beads have first and second capture agents immobilized thereon, respectively; and
xv. the first and second capture agents are capable of binding to and immobilizing the first and second analytes, respectively;
   wherein in the open configuration, the two plates are partially or entirely separated apart, the spacing between the plates is not regulated by the spacers, and the sample is deposited on one or both of the plates; and
   wherein in the closed configuration, which is configured after deposition of the sample in the open configuration: at least part of the sample is compressed by the two plates into a layer of highly uniform thickness, the uniform thickness of the layer is confined by the inner surfaces of the plates and is regulated by the plates and the spacers, the analytes in the layer of uniform thickness are concentrated by the beads so that signal of the captured analytes on the beads is distinguishable from signal emanating from other area in the layer of uniform thickness.
      NB1. A smartphone system for rapid multiplexed homogeneous assay, comprising:

(a) a device of embodiment NA1;

A method of performing a rapid homogeneous assay, comprising the steps of:

(a) obtaining a sample suspected of containing a first analyte of one species and a second analyte of a different species;

(b) obtaining a first plate and a second plate, wherein:
i. the plates are movable relative to each other into different configurations, including an open configuration and a closed configuration;
ii. each of the plates has, on its respective inner surface, a sample contact area for contacting the sample;
iii. one or both of the plates comprise spacers, at least one of the spacers is inside the sample contact area, and the spacers have a predetermined substantially uniform height;
iv. one or both of the plates comprise, on the respective inner surface, a plurality of first beads and second beads, wherein the first and second beads have first and second capture agents immobilized thereon, respectively; and
v. the first and second capture agents are capable of binding to and immobilizing the first and second analyte, respectively;

(c) depositing the sample on one or both of the plates when the plates in the open configuration, wherein in the open configuration, the two plates are partially or entirely separated apart and the spacing between the plates is not regulated by the spacers;

(d) after (c), bringing the two plates together and pressing the plates into the closed configuration, wherein in the closed configuration: at least part of the sample is compressed by the two plates into a layer of highly uniform thickness, the uniform thickness of the layer is confined by the inner surfaces of the two plates and is regulated by the spacers and the plates; and

(e) while the plates are at the closed configuration, detecting and analyzing the analytes in the layer of uniform thickness.

NA2. The device, smartphone system, and method of any prior embodiments, wherein the first and second beads are different.
NA3. The device, smartphone system, and method of any prior embodiments, wherein the first and second beads are different in their sizes.
NA4. The device, smartphone system, and method of any prior embodiments, wherein the first and second beads are different in their optical properties selected from the group consisting of: photoluminescence, electroluminescence, and electrochemiluminescence, light absorption, reflection, transmission, diffraction, scattering, diffusion, surface Raman scattering, and any combination thereof.
NA5. The device, smartphone system, and method of any prior embodiments, wherein the first and second beads are different in their electric densities.
NA4. The device, smartphone system, and method of any prior embodiments, wherein the first and second beads are the same, and wherein the signals from the first and second analytes are differentmaking plate with periodically arranged beads
ND1. A method of making a plate with periodically arranged beads, comprising the steps of:
(1) having a plate that comprises a plurality of pits on its inner surface, wherein the pits are periodically arranged;
(2) depositing a liquid that contains a plurality of beads on the inner surface of the plate; and
(3) drying the plate, during which process the beads are re-distributed inside the pits due to at least the capillary force on the ridge of the pits.

### Analyte Concentration Area:

AA1-1. A device for rapid homogeneous assay, comprising:

a first plate, a second plate, and spacers, wherein:
i. the plates are movable relative to each other into different configurations, including an open configuration and a closed configuration;
ii. each of the plates has, on its respective inner surface, a sample contact area for contacting a sample suspected of comprising an analyte;
iii. one or both of the plates comprise the spacers, at least one of the spacers is inside the sample contact area, and the spacers have a predetermined substantially uniform height; and
iv. one or both of the plates comprise, on the respective inner surface, one or a plurality of analyte concentration areas that have capture agent immobilized thereon, wherein the capture agent is capable of binding to and immobilizing the analyte;
   wherein in the open configuration, the two plates are partially or entirely separated apart, the spacing between the plates is not regulated by the spacers, and the sample is deposited on one or both of the plates; and
   wherein in the closed configuration, which is configured after deposition of the sample in the open configuration: at least part of the sample is compressed by the two plates into a layer of highly uniform thickness, the uniform thickness of the layer is confined by the inner surfaces of the plates and is regulated by the plates and the spacers and the analyte in the layer of uniform thickness is concentrated in the analyte concentration area so that signal of captured analyte in the analyte concentration areas is distinguishable from signal emanating from non-analyte concentration area in the layer of uniform thickness.

### Concentration Protrusion:

AA2. The device of any prior embodiment, wherein one or both of the plates comprise one or a plurality of protrusions extending from the respective inner surface, and wherein each protrusion has a height smaller than the spacers and comprises the analyte concentration area on at least one of its surfaces.

### Beads:

AB1. A device for rapid homogeneous assay, comprising:

a first plate, a second plate, and spacers, wherein:
i. the plates are movable relative to each other into different configurations, including an open configuration and a closed configuration;
ii. each of the plates has, on its respective inner surface, a sample contact area for contacting a sample suspected of comprising an analyte;
iii. one or both of the plates comprise the spacers, at least one of the spacers is inside the sample contact area, and the spacers have a predetermined substantially uniform height; and
iv. one or both of the plates comprise, on the respective inner surface, a plurality of beads that have capture agent immobilized thereon, wherein the capture agent is capable of binding to and immobilizing the analyte;
   wherein in the open configuration, the two plates are partially or entirely separated apart, the spacing between the plates is not regulated by the spacers, and the sample is deposited on one or both of the plates; and
   wherein in the closed configuration, which is configured after deposition of the sample in the open configuration: at least part of the sample is compressed by the two plates into a layer of highly uniform thickness, the uniform thickness of the layer is confined by the inner surfaces of the plates and is regulated by the plates and the spacers, the analyte in the layer of uniform thickness is concentrated by the beads so that signal of the captured analyte on the beads is distinguishable from signal emanating from other area in the layer of uniform thickness.

### System:

C1. A system for rapid homogeneous assay, comprising:

(a) a device of any prior embodiment; and

(b) a detector that detects signals from the capture agent-bound analyte indicative of the presence and/or quantity of the analyte in the layer of uniform thicknessSmartphone System:

D1. A smartphone system for rapid homogeneous assay, comprising:
(a) a device of any prior embodiment;
(b) a mobile communication device that comprises:
   i. one or a plurality of cameras for detecting and/or imaging the sample;
   ii. electronics, signal processors, hardware and software for receiving and/or processing the detected signal and/or the image of the sample and for remote communication; and
(c) an adaptor that is configured to hold the closed device and engageable to mobile communication device;

wherein when engaged with the mobile communication device, the adaptor is configured to facilitate the detection and/or imaging of the analyte in the sample at the closed configuration.

### Method:

AE1. A method of performing a rapid homogeneous assay, comprising the steps of:

(a) obtaining a sample suspected of containing an analyte;

(b) obtaining a device of any prior embodiment;

(c) depositing the sample on one or both of the plates when the plates are in the open configuration;

(d) after (c), bringing the two plates together and pressing the plates into the closed configuration; and

(e) while the plates are at the closed configuration, detecting and analyzing the analyte in the layer of uniform thickness.

AE2. A method of analyzing the image for a rapid homogeneous assay, comprising the steps of:

(a) obtaining an image of the signal in any prior embodiment at the closed configuration, wherein the image is selected from the group consisting of bright field image, dark field image, fluorescence image, and phosphorescence image;

(b) analyzing the image, identifying beads in the image, and extracting information of beads size, signal intensity of beads, distance between beads, distribution of beads, and number of beads; and

(c) deducing analyte concentration by analyzing the extracted information from step (b) and calculating parameters of the beads.

E1. A method of performing a homogeneous assay, comprising the steps of:

(a) obtaining a sample suspected of containing an analyte;

(b) obtaining a first and second plates that are movable relative to each other into different configurations, including an open configuration and a closed configuration, wherein:
i. each of the plates has, on its respective inner surface, a sample contact area for contacting the sample,
ii. one or both of the plates comprise the spacers, and at least one of the spacers is inside the sample contact area;
iii. one or both of the plates comprise, on the respective inner surface, a plurality of beads that have capture agent immobilized thereon, wherein the capture agent is capable of binding to and immobilizing the analyte; and
iv. one or both of the plates comprise, on the respective inner surface, detection agent that is configured to, upon contacting the sample, be dissolved in the sample and bind to the analyte;
   wherein the spacers have a predetermined substantially uniform height;

(c) depositing the sample on one or both of the plates when the plates are in an open configuration, wherein in the open configuration the two plates are partially or entirely separated apart and the spacing between the plates is not regulated by the spacers;

(d) after (c), bringing the two plates together and pressing the plates into a closed configuration, wherein in the closed configuration: at least part of the sample is compressed by the two plates into a layer of highly uniform thickness, the uniform thickness of the layer is confined by the inner surfaces of the two plates and is regulated by the spacers and the plates; and

(e) while the plates are at the closed configuration, detecting and analyzing the analyte in the layer of uniform thickness,
wherein the capture agent and the detection agent are configured to bind to the analyte at different locations thereof and to form a capture agent-analyte-detection agent sandwich that is immobilized to the bead; and
wherein the beads, the capture agent, and the detection agent are configured to render signal from the bead-associated capture agent-analyte-detection agent sandwich distinguishable from signal of free detection agent in the layer of uniform thickness.

### Embodiments Defining Diffusion Parameters

AA1-2. A device for rapid homogeneous assay, comprising:

a first plate, a second plate, and spacers, wherein:
i. the plates are movable relative to each other into different configurations, including an open configuration and a closed configuration;
ii. each of the plates has, on its respective inner surface, a sample contact area for contacting a sample suspected of comprising an analyte;
iii. one or both of the plates comprise the spacers, at least one of the spacers is inside the sample contact area, and the spacers have a predetermined substantially uniform height of 200 um or less; and
iv. one or both of the plates comprise, on the respective inner surface, one or a plurality of analyte concentration areas that has capture agent immobilized thereon, wherein the capture agent is capable of binding the analyte;
   wherein the spacers have a height that is equal to or less than 4 times of a diffusion parameter, wherein the diffusion parameter is square root of the intended assay time multiplying diffusion constant of the analyte in the sample and wherein the intended assay time is equal to or less than 240 seconds;
   wherein the average distance between two neighboring analyte concentration areas is equal to or less than 4 times of the diffusion parameter;
   wherein in the open configuration, the two plates are partially or entirely separated apart, the spacing between the plates is not regulated by the spacers, and the sample is deposited on one or both of the plates; and
   wherein in the closed configuration, which is configured after deposition of the sample in the open configuration: at least part of the sample is compressed by the two plates into a layer of highly uniform thickness, the uniform thickness of the layer is confined by the inner surfaces of the plates and is regulated by the plates and the spacers and the analyte in the layer of uniform thickness is concentrated in the concentration area so that signal of captured analyte in the concentration areas is distinguishable from signal emanating from non-concentration area in the layer of uniform thickness.
      AA2-2. The device of any prior embodiment, wherein one or both of the plates comprise one or a plurality of protrusions extending from the respective inner surface, and wherein each protrusion has a height smaller than the spacers and comprises the analyte concentration area on at least one of its surfaces.
      AB1-2. A device for rapid homogeneous assay, comprising:

a first plate, a second plate, and spacers, wherein:
i. the plates are movable relative to each other into different configurations, including an open configuration and a closed configuration;
ii. each of the plates has, on its respective inner surface, a sample contact area for contacting a sample suspected of comprising an analyte;
iii. one or both of the plates comprise the spacers, at least one of the spacers is inside the sample contact area, and the spacers have a predetermined substantially uniform height; and
iv. one or both of the plates comprise, on the respective inner surface, a plurality of beads that have capture agent immobilized thereon, wherein the capture agent is capable of binding to and immobilizing the analyte;
   wherein the spacers have a height that is equal to or less than 3 times of a diffusion parameter, wherein the diffusion parameter is square root of the intended assay time multiplying diffusion constant of the analyte in the sample and wherein the intended assay time is equal to or less than 240 seconds;
   wherein the average distance between two neighboring beads is equal to or less than 2 times of the diffusion parameter;
   wherein in the open configuration, the two plates are partially or entirely separated apart, the spacing between the plates is not regulated by the spacers, and the sample is deposited on one or both of the plates; and
   wherein in the closed configuration, which is configured after deposition of the sample in the open configuration: at least part of the sample is compressed by the two plates into a layer of highly uniform thickness, the uniform thickness of the layer is confined by the inner surfaces of the plates and is regulated by the plates and the spacers and the analyte in the layer of uniform thickness is concentrated in the concentration area so that signal of captured analyte in the concentration areas is distinguishable from signal emanating from non-concentration area in the layer of uniform thickness.
      AE1-2. A method of performing a rapid homogeneous assay, comprising the steps of:

(a) obtaining a sample suspected of containing an analyte;

(b) obtaining a device of any prior embodiment;

(c) depositing the sample on one or both of the plates when the plates are in the open configuration;

(d) after (c), bringing the two plates together and pressing the plates into the closed configuration; and

(e) after step (d), incubating the assay for a time equal to or longer than the intended assay time, detecting and analyzing the analyte in the layer of uniform thickness.

DP1. The device, kit, system, smartphone system, and method of any prior embodiments, wherein the intended assay time is in the range of 0.1-240 sec.
DP2-1. The device, kit, system, smartphone system, and method of any prior embodiments, wherein the intended assay time is in the range of 1-60 sec.
DP2-2. The device, kit, system, smartphone system, and method of any prior embodiments, wherein the intended assay time is equal to or less than 30 sec.
DP2-3. The device, kit, system, smartphone system, and method of any prior embodiments, wherein the intended assay time is equal to or less than 10 sec.
DP2-4. The device, kit, system, smartphone system, and method of any prior embodiments, wherein the intended assay time is equal to or less than 5 sec.
DP-5. The device, kit, system, smartphone system, and method of any prior embodiments, wherein the intended assay time is equal to or less than 1 sec.
DP3. The device, kit, system, smartphone system, and method of any prior embodiments, wherein the average distance between two neighboring analyte concentration areas or beads is in the range of 50 nm-200 um.
DP4-1. The device, kit, system, smartphone system, and method of any prior embodiments, wherein the average distance between two neighboring analyte concentration areas or beads is in the range of 500 nm-20 um.
DP4-2. The device, kit, system, smartphone system, and method of any prior embodiments, wherein the average distance between two neighboring analyte concentration areas or beads is in the range of 500 nm-10 urn.
DP4-3. The device, kit, system, smartphone system, and method of any prior embodiments, wherein the average distance between two neighboring analyte concentration areas or beads is in the range of 500 nm-5 urn.
DP5. The device, kit, system, smartphone system, and method of any prior embodiments, wherein the ratio of the spacers' height versus the diffusion parameter is in the range of 0.01-2.
DP6-1. The device, kit, system, smartphone system, and method of any prior embodiments, wherein the ratio of the spacers' height versus the diffusion parameter is in the range of 0.1-1.5.
DP6-2. The device, kit, system, smartphone system, and method of any prior embodiments, wherein the ratio of the spacers' height versus the diffusion parameter is in the range of 0.01-0.5.
DP6-3. The device, kit, system, smartphone system, and method of any prior embodiments, wherein the ratio of the spacers' height versus the diffusion parameter is in the range of 0.01-0.2.
DP6-4. The device, kit, system, smartphone system, and method of any prior embodiments, wherein the ratio of the spacers' height versus the diffusion parameter is in the range of 0.01-0.1.
DP7. The device, kit, system, smartphone system, and method of any prior embodiments, wherein the ratio of the average distance between two neighboring analyte concentration areas or beads versus the diffusion parameter is in the range of 0.01-5.
DP8-1. The device, kit, system, smartphone system, and method of any prior embodiments, wherein the ratio of the average distance between two neighboring analyte concentration areas or beads versus the diffusion parameter is in the range of 0.01-1.5.
DP8-2. The device, kit, system, smartphone system, and method of any prior embodiments, wherein the ratio of the average distance between two neighboring analyte concentration areas or beads versus the diffusion parameter is in the range of 0.01-1.
DP8-3. The device, kit, system, smartphone system, and method of any prior embodiments, wherein the ratio of the average distance between two neighboring analyte concentration areas or beads versus the diffusion parameter is in the range of 0.01-0.5.
DP8-4. The device, kit, system, smartphone system, and method of any prior embodiments, wherein the ratio of the average distance between two neighboring analyte concentration areas or beads versus the diffusion parameter is in the range of 0.01-0.2.
DP8-5. The device, kit, system, smartphone system, and method of any prior embodiments, wherein the ratio of the average distance between two neighboring analyte concentration areas or beads versus the diffusion parameter is in the range of 0.01-0.1.
DP9-1. The device, kit, system, smartphone system, and method of any prior embodiments, wherein the ratio of the average distance between two neighboring analyte concentration areas or beads versus the diffusion parameter is in the range of 0.01-0.5, and the ratio of the spacers' height versus the diffusion parameter is in the range of 0.01-0.2.
DP9-2. The device, kit, system, smartphone system, and method of any prior embodiments, wherein the ratio of the average distance between two neighboring analyte concentration areas or beads versus the diffusion parameter is in the range of 0.01-1, and the ratio of the spacers' height versus the diffusion parameter is in the range of 0.01-0.5.
DP9-3. The device, kit, system, smartphone system, and method of any prior embodiments, wherein the ratio of the average distance between two neighboring analyte concentration areas or beads versus the diffusion parameter is in the range of 0.01-2, and the ratio of the spacers' height versus the diffusion parameter is in the range of 0.01-1.
DP9-4. The device, kit, system, smartphone system, and method of any prior embodiments, wherein the ratio of the average distance between two neighboring analyte concentration areas or beads versus the diffusion parameter is in the range of 0.01-4, and the ratio of the spacers' height versus the diffusion parameter is in the range of 0.01-1.
DP10-1. The device, kit, system, smartphone system, and method of any prior embodiments, wherein the ratio of the average distance between two neighboring analyte concentration areas or beads versus the diffusion parameter is in the range of 0.01-0.5, the ratio of the spacers' height versus the diffusion parameter is in the range of 0.01-0.2, and the intended assay time is equal to or less than 120 sec.
DP10-2. The device, kit, system, smartphone system, and method of any prior embodiments, wherein the ratio of the average distance between two neighboring analyte concentration areas or beads versus the diffusion parameter is in the range of 0.01-1; the ratio of the spacers' height versus the diffusion parameter is in the range of 0.01-0.5, and the intended assay time is equal to or less than 60 sec.
DP10-3. The device, kit, system, smartphone system, and method of any prior embodiments, wherein the ratio of the average distance between two neighboring analyte concentration areas or beads versus the diffusion parameter is in the range of 0.01-2; the ratio of the spacers' height versus the diffusion parameter is in the range of 0.01-1; and the intended assay time is equal to or less than 30 sec.
DP10-4. The device, kit, system, smartphone system, and method of any prior embodiments, wherein the ratio of the average distance between two neighboring analyte concentration areas or beads versus the diffusion parameter is in the range of 0.01-4; the ratio of the spacers' height versus the diffusion parameter is in the range of 0.01-1; and the intended assay time is equal to or less than 30 sec.

### More:

### (Sandwich Assay)

AA1 The device, kit, system, smartphone system, and method of any prior embodiments, wherein the analyte is labeled by detection agent that selectively binds to the analyte and is associated with a label.
AA1.1 The device, kit, system, smartphone system, and method of any prior embodiments, wherein the detection agent is coated on the inner surface(s) of one or both of the plates, and is configured to, upon contacting the sample, be dissolved and diffuse in the sample.
AA1.2 The device, kit, system, smartphone system, and method of any prior embodiments, wherein the detection agent is pre-loaded into the sample before the sample is deposited on the plate(s).
AA1.3 The device, kit, system, smartphone system, and method of any prior embodiments, wherein the capture agent and the detection agent are configured to bind to the analyte at different locations thereof and form capture agent-analyte-detection agent sandwich.

### (Competitive Assay)

AA2 The device, kit, system, smartphone system, and method of any prior embodiments, wherein the analyte competes with a detection agent to bind to the capture agent, and wherein the detection agent is labeled.
AA3 The device, kit, system, smartphone system, and method of any prior embodiments, wherein one or both of the plates comprise, on the respective inner surface, a signal amplification surface that amplify the signal in proximity to the amplification surface.
A2 The device, kit, system, smartphone system, and method of any prior embodiments, wherein the beads are the spacers that regulate the thickness of the layer at the closed configuration.
A2.1 The device, kit, system, smartphone system, and method of any prior embodiments, wherein the beads are micro- or nano-particles having an average diameter in the range of 1 nm to 200 um.
AA2.1 The device, kit, system, smartphone system, and method of any prior embodiments, wherein the analyte concentration areas have an average diameter in the range of 1 nm to 200 um.
AAA2.1 The device, kit, system, smartphone system, and method of any prior embodiments, wherein the concentrating protrusions have an average diameter in the range of 1 nm to 200 um.
A2.1.1 The device, kit, system, smartphone system, and method of any prior embodiments, wherein the beads have an average diameter in the range of 0.1 μm to 10 μm.
A2.1.2 The device, kit, system, smartphone system, and method of any prior embodiments, wherein the beads have an average diameter in the range of 1 nm to 500 nm.
A2.1.3 The device, kit, system, smartphone system, and method of any prior embodiments, wherein the beads have an average diameter in the range of to 0.5 μm to 30 μm.
A2.1.4 The device, kit, system, smartphone system, and method of any prior embodiments, wherein ratio between the spacing between the plates at the closed configuration and average dimeter of the beads is in the range of 1-100.
AA2.1.4 The device, kit, system, smartphone system, and method of any prior embodiments, wherein ratio between the spacing between the plates at the closed configuration and height of the analyte concentration area is in the range of 1-100.
AAA2.1.4 The device, kit, system, smartphone system, and method of any prior embodiments, wherein ratio between the spacing between the plates at the closed configuration and height of the concentrating protrusion is in the range of 1-100.
A2.2 The device, kit, system, smartphone system, and method of any prior embodiments, wherein the beads have an area density of 1 to 10⁶ per mm².
AA2.2 The device, kit, system, smartphone system, and method of any prior embodiments, wherein the analyte concentration areas have an area density of 1 to 10⁶ per mm².
AAA2.2 The device, kit, system, smartphone system, and method of any prior embodiments, wherein the concentrating protrusions have an area density of 1 to 10⁶ per mm².
A2.2.1 The device, kit, system, smartphone system, and method of any prior embodiments, wherein the beads have an area density of 1 to 1000 per mm².
A2.3 The device, kit, system, smartphone system, and method of any prior embodiments, wherein the beads are configured to amplify the signal in proximity to the beads, and have a signal amplification factor in the range of 1 to 10000.
A2.4 The device, kit, system, smartphone system, and method of any prior embodiments, wherein the detection antibody is configured to have a concentration in the layer of uniform thickness that is 1 to 1000 times higher than analyte concentration in the sample.
A3 The device, kit, system, smartphone system, and method of any prior embodiments, wherein the beads and the detection agent are on the same plate.
A3.1 The device, kit, system, smartphone system, and method of any prior embodiments, wherein the beads and the detection agent are on different plates.
A4 The device, kit, system, smartphone system, and method of any prior embodiments, wherein the analyte is selected from the group consisting of: cells, viruses, proteins, peptides, DNAs, RNAs, oligonucleotides, and any combination thereof.
A4.1 The device, kit, system, smartphone system, and method of any prior embodiments, wherein the analyte is C Reactive Protein (CRP).
A5.1 The device, kit, system, smartphone system, and method of any prior embodiments, wherein the capture agent is selected from the group consisting of: protein, peptide, peptidomimetics, streptavidin, biotin, oligonucleotide, oligonucleotide mimetics, any other affinity ligand and any combination thereof.
A5.1.1 The device, kit, system, smartphone system, and method of any prior embodiments, wherein the capture agent is an antibody.
A5.1.2 The device, kit, system, smartphone system, and method of any prior embodiments, wherein the capture antibody is an anti-C Reactive Protein (CRP) antibody.
A5.1.3 The device, kit, system, smartphone system, and method of any prior embodiments, wherein the capture agent has a concentration that is sufficient to detect the presence and/or measure the amount of the analyte.
A5.1.4 The device, kit, system, smartphone system, and method of any prior embodiments, wherein the capture agent has a concentration that is sufficient to immobilize the analyte.
A5.2 The device, kit, system, smartphone system, and method of any prior embodiments, wherein the detection agent is selected from the group consisting of: protein, peptide, peptidomimetics, streptavidin, biotin, oligonucleotide, oligonucleotide mimetics, any other affinity ligand and any combination thereof.
A5.2.1 The device, kit, system, smartphone system, and method of any prior embodiments, wherein the detection agent is an antibody.
A5.2.2 The device, kit, system, smartphone system, and method of any prior embodiments, wherein the detection antibody is an anti-CRP antibody.
A6 The device, kit, system, smartphone system, and method of any prior embodiments, wherein the beads are made of a material selected from the group consisting of: polysteryne, polypropylene, polycarbonate, PMMG, PC, COC, COP, glass, resin, aluminum, gold or other metal or any other material whose surface can be modified to be associated with the capture agent.
A6.1 The device, kit, system, smartphone system, and method of any prior embodiments, wherein the beads are treated with a protein stabilizer.
A6.2 The device, kit, system, smartphone system, and method of any prior embodiments, wherein the capture agent are conjugated with the beads.
A6.3 The device, kit, system, smartphone system, and method of any prior embodiments, wherein the beads are prepared by:

(d) activating with N-Hydroxysuccinimide (NHS);

(e) blocking with a BSA solution; and

(f) incubating with a capture agent solution.

A7 The device, kit, system, smartphone system, and method of any prior embodiments, wherein the liquid sample is made from a biological sample selected from the group consisting of: amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, urine, and any combination thereof.
A7.1 The device, kit, system, smartphone system, and method of any prior embodiments, wherein the sample is an environmental liquid sample from a source selected from the group consisting of: river, lake, pond, ocean, glaciers, icebergs, rain, snow, sewage, reservoirs, tap water, or drinking water, solid samples from soil, compost, sand, rocks, concrete, wood, brick, sewage, and any combination thereof.
A7.2 The device, kit, system, smartphone system, and method of any prior embodiments, wherein the sample is an environmental gaseous sample from a source selected from the group consisting of: the air, underwater heat vents, industrial exhaust, vehicular exhaust, and any combination thereof.
A7.3 The device, kit, system, smartphone system, and method of any prior embodiments, wherein the sample is a foodstuff sample selected from the group consisting of: raw ingredients, cooked food, plant and animal sources of food, preprocessed food, and partially or fully processed food, and any combination thereof.
A8. The device, kit, system, smartphone system, and method of any prior embodiments, wherein the detection agent is a labeled agent.
A8.1 The device, kit, system, smartphone system, and method of any prior embodiments, wherein the detection agent is labeled with a fluorophore.
A8.1.1 The device, kit, system, smartphone system, and method of any prior embodiments, wherein the fluorophore is Cy5.

### (Quencher)

A8.2 The device, kit, system, smartphone system, and method of any prior embodiments, wherein the beads are associated with a label, and wherein the detection agent is a quencher that is configured to quench signal of the beads-associated label when the detection agent is in proximity of the label.
A9. The device, kit, system, smartphone system, and method of any prior embodiments, wherein the detector detects the signal emanating from the analyte concentration areas or beads indicative of the presence and/or quantity of the analyte.
A9.1 The device, kit, system, smartphone system, and method of any prior embodiments, wherein the signal is:
i. luminescence selected from photoluminescence, electroluminescence, and electrochemiluminescence;
ii. light absorption, reflection, transmission, diffraction, scattering, or diffusion;
iii. surface Raman scattering;
iv. electrical impedance selected from resistance, capacitance, and inductance;
v. magnetic relaxivity; or
vi. any combination of i-v.
   D2. The smartphone system of any prior embodiments, wherein the mobile communication device is configured to communicate test results to a medical professional, a medical facility or an insurance company.
   D3. The smartphone system of any prior embodiments, wherein the mobile communication device is further configured to communicate information on the subject with the medical professional, medical facility or insurance company.
   D4. The smartphone system of any prior embodiments, wherein the mobile communication device is configured to receive a prescription, diagnosis or a recommendation from a medical professional.
   D5. The smartphone system of any prior embodiments, wherein the mobile communication device communicates with the remote location via a wifi or cellular network.
   D6. The smartphone system of any prior embodiments, wherein the mobile communication device is a mobile phone.
   E2 The method of any prior embodiments, wherein the sample contact sites are not washed before the imaging step (e).
   E3 The method of any of embodiments 1-5, further comprising washing the sample contact area before the imaging step (e).
   E4 The method of any prior embodiments, further comprising determining the presence of the analyte and/or measuring the amount of the analyte.
   AE2.1 The method of embodiment AE2, wherein the calculated parameters comprise average signal intensity from all the beads that are analyzed.
   AE2.2 The method of embodiment AE2, wherein the calculated parameters comprise highest signal intensity from all the beads that are analyzed.
   AE2.3 The method of embodiment AE2, wherein the calculated parameters comprise signal intensity distribution from all the beads that are analyzed.
   AE2.4 The method of embodiment AE2, wherein the calculated parameters comprise number of all the beads that are analyzed with signal intensity larger than a threshold;
   AE2.5 The method of embodiment AE2, wherein the calculated parameters comprise average signal intensity from all the beads that are analyzed in a first area of the image.
   AE2.6 The method of embodiment AE2, wherein the calculated parameters comprise highest signal intensity from all the beads that are analyzed in a first area of the image.
   AE2.7 The method of embodiment AE2, wherein the calculated parameters comprise signal intensity distribution from all the beads that are analyzed in a first area of the image.
   AE2.8 The method of embodiment AE2, wherein the calculated parameters comprise number of all the beads that are analyzed in a first area of the image with signal intensity larger than a threshold.
   F1 The device that comprises two plates and spacers, wherein the pressing is by human hand.
   F2 The device that comprises two plates and spacers, wherein at least a portion of the inner surface of one plate or both plate is hydrophilic.
   F3 The device that comprises two plates and spacers, wherein the inter spacer distance is periodic.
   F4 The device that comprises two plates and spacers, wherein the sample is a deposition directly from a subject to the plate without using any transferring devices.
   F5 The device that comprises two plates and spacers, wherein after the sample deformation at a closed configuration, the sample maintains the same final sample thickness, when some or all of the compressing forces are removed.
   F6 The device that comprises two plates and spacers, wherein the spacers have pillar shape and nearly uniform cross-section.
   F7 The device that comprises two plates and spacers, wherein the inter spacer distance (SD) is equal or less than about 120 um (micrometer).
   F8 The device that comprises two plates and spacers, wherein the inter spacer distance (SD) is equal or less than about 100 um (micrometer).
   F9 The device that comprises two plates and spacers, wherein the fourth power of the inter-spacer-distance (ISD) divided by the thickness (h) and the Young's modulus (E) of the flexible plate (ISD\hat{ }4/(hE)) is 5×10\hat{ }6 um\hat{ }3/GPa or less.
   F10 The device that comprises two plates and spacers, wherein the fourth power of the inter-spacer-distance (ISD) divided by the thickness (h) and the Young's modulus (E) of the flexible plate (ISD\hat{ }4/(hE)) is 5×10\hat{ }5 um3/GPa or less.
   F11 The device that comprises two plates and spacers, wherein the spacers have pillar shape, a substantially flat top surface, a predetermined substantially uniform height, and a predetermined constant inter-spacer distance that is at least about 2 times larger than the size of the analyte, wherein the Young's modulus of the spacers times the filling factor of the spacers is equal or larger than 2 MPa, wherein the filling factor is the ratio of the spacer contact area to the total plate area, and wherein, for each spacer, the ratio of the lateral dimension of the spacer to its height is at least 1 (one).
   F12 The device that comprises two plates and spacers, wherein the spacers have pillar shape, a substantially flat top surface, a predetermined substantially uniform height, and a predetermined constant inter-spacer distance that is at least about 2 times larger than the size of the analyte, wherein the Young's modulus of the spacers times the filling factor of the spacers is equal or larger than 2 MPa, wherein the filling factor is the ratio of the spacer contact area to the total plate area, and wherein, for each spacer, the ratio of the lateral dimension of the spacer to its height is at least 1 (one), wherein the fourth power of the inter-spacer-distance (ISD) divided by the thickness (h) and the Young's modulus (E) of the flexible plate (ISD\hat{ }4/(hE)) is 5×10\hat{ }6 um\hat{ }3/GPa or less.
   F13 The device of any prior device claim, wherein the ratio of the inter-spacing distance of the spacers to the average width of the spacer is 2 or larger, and the filling factor of the spacers multiplied by the Young's modulus of the spacers is 2 MPa or larger.
   F14 The device, kit, system, smartphone system, and method of any prior embodiments wherein the analytes is the analyte in 5 detection of proteins, peptides, nucleic acids, synthetic compounds, and inorganic compounds.
   F15 The device, kit, system, smartphone system, and method of any prior embodiments wherein the sample is a biological sample selected from amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, and urine.
   F16 The device, kit, system, smartphone system, and method of any prior embodiments wherein the spacers have a shape of pillars and a ratio of the width to the height of the pillar is equal or larger than one.
   F17 The method of any prior claim, wherein the sample that is deposited on one or both of the plates has an unknown volume.
   F18 The device, kit, system, smartphone system, and method of any prior embodiments wherein the spacers have a shape of pillar, and the pillar has substantially uniform cross-section.
   F19 The device, kit, system, smartphone system, and method of any prior embodiments wherein the samples are for the detection, purification and quantification of chemical compounds or biomolecules that correlates with the stage of certain diseases.
   F20 The device, kit, system, smartphone system, and method of any prior embodiments wherein the samples is related to infectious and parasitic disease, injuries, cardiovascular disease, cancer, mental disorders, neuropsychiatric disorders, pulmonary diseases, renal diseases, and other and organic diseases.
   F21 The device, kit, system, smartphone system, and method of any prior embodiments wherein the samples are related to the detection, purification and quantification of microorganism.
   F22 The device, kit, system, smartphone system, and method of any prior embodiments wherein the samples is related to virus, fungus and bacteria from environment, e.g., water, soil, or biological samples.
   F23 The device, kit, system, smartphone system, and method of any prior embodiments wherein the samples is related to the detection, quantification of chemical compounds or biological samples that pose hazard to food safety or national security, e.g. toxic waste, anthrax.
   F24 The device, kit, system, smartphone system, and method of any prior embodiments wherein the samples are related to quantification of vital parameters in medical or physiological monitor.
   F25 The device, kit, system, smartphone system, and method of any prior embodiments wherein the samples are related to glucose, blood, oxygen level, total blood count.
   F26 The device, kit, system, smartphone system, and method of any prior embodiments wherein the samples are related to the detection and quantification of specific DNA or RNA from biosamples.
   F27 The device, kit, system, smartphone system, and method of any prior embodiments wherein the samples are related to the sequencing and comparing of genetic sequences in DNA in the chromosomes and mitochondria for genome analysis.
   F28 The device, kit, system, smartphone system, and method of any prior embodiments wherein the samples are related to detect reaction products, e.g., during synthesis or purification of pharmaceuticals.
   F29 The device, kit, system, smartphone system, and method of any prior embodiments wherein the samples are cells, tissues, bodily fluids, and stool.
   F30 The device, kit, system, smartphone system, and method of any prior embodiments wherein the sample is the sample in the detection of proteins, peptides, nucleic acids, synthetic compounds, inorganic compounds.
   F31 The device, kit, system, smartphone system, and method of any prior embodiments wherein the sample is the sample in the fields of human, veterinary, agriculture, foods, environments, and drug testing.
   F32 The method or device of any prior claim, wherein the sample is a biological sample .is selected from blood, serum, plasma, a nasal swab, a nasopharyngeal wash, saliva, urine, gastric fluid, spinal fluid, tears, stool, mucus, sweat, earwax, oil, a glandular secretion, cerebral spinal fluid, tissue, semen, vaginal fluid, interstitial fluids derived from tumorous tissue, ocular fluids, spinal fluid, a throat swab, breath, hair, finger nails, skin, biopsy, placental fluid, amniotic fluid, cord blood, lymphatic fluids, cavity fluids, sputum, pus, microbiota, meconium, breast milk, exhaled condensate nasopharyngeal wash, nasal swab, throat swab, stool samples, hair, finger nail, ear wax, breath, connective tissue, muscle tissue, nervous tissue, epithelial tissue, cartilage, cancerous sample, or bone.

### Example-1

### Homogeneous QMAX Immunoassay—for Human CRP (C-Reactive Protein)

Here we describe an experiment of homogeneous QMAX immunoassay for human CRP according to one embodiment of the present invention.

In this experiment, the device for the immunoassay comprises a first plate and a second plate. Conventional glass slide was used as the first plate and X-plate with 10 urn spacer as the second plate. The microbeads were coated on the first plate, and the microbeads (Pierce, 10 μm in diameter) were NHS activated and conjugated to capture antibody (anti-CRP mouse monoclonal, Fitzgerald). A fluorescence microscope was used as the detector. The average distance between two neighboring beads is about 30 um to 50 um.

The experiment was conducted according to the following procedures:
1. Conjugation of capture antibody to beads. NHS activated beads (Pierce, 10 μm in diameter) were conjugated to anti-CRP mouse monoclonal capture antibody (Fitzgerald) according to manufacturer's protocol.
2. Blocking of beads. The antibody conjugated beads were blocked by 4% BSA in PBS at 4° C. over night and washed by PBST for 6 times prior to use.
3. Coating first plate. 1 pL of beads from Step 2 (beads concentration 10⁷-10⁸/mL) were dropped on glass slide (Fisher Scientific) and air dried at room temperature.
4. Homogeneous QMAX assay. 1 pL of CRP analyte (Fitzgerald) at the concentration of 10 μg/mL and 1 μL of Cy5-labeled anti-CRP mouse monoclonal detection antibody (Fitzgerald) were dropped onto the area of coated beads on the glass slide. Different concentrations of Cy5 labeled anti-CRP detection antibody (A, 800 μg/mL; B, 100 μg/mL; C, 50 μg/mL; D, 25 μg/mL and E, 0 μg/mL) were tested separately. The mixture was immediately covered by X-plate (second plate) with 10 μm spacer and incubated for 30 seconds at room temperature.
5. Imaging. Without washing, the fluorescent images were taken by the fluorescence microscope (Ex 640 nm, Em 670-690 nm).

### Example-2

### BEADS-Enhanced Speedy Test (BEST) Structure Examples

1. Exemplary embodiments of Beads-Enhanced Speedy Test (BEST)—Beads based:

One exemplary device comprises a first plate, a second plate, an array of spacers on the second plate, beads and concentration areas.

First plate: 24 mm×32 mm size, 1 mm thick plastic (as acrylic or polystyrene) or glass

Second plate: 22 mm×25 mm size, 175 um thick plastic (as acrylic or polystyrene) with an array of pillar spacers on one side. The pillar spacers are 30×40 um in lateral size, and 10 um in height, and the inter-spacer distance is 80 um for the array.

Beads: 10 um in diameter plastic beads (as acrylic or polystyrene) with an area concentration of 100/mm2 to 1000/mm2, which are uniformly pre-dried on the second plate.

Concentration areas: on the surface of all the beads

### 2. Exemplary Embodiments of Beads-Enhanced Speedy Test (BEST)—Beads Based:

Another exemplary device comprises a first plate, a second plate, spacer array on the second plate, beads and concentration areas.

First plate: 24 mm×32 mm size, 1 mm thick plastic (as acrylic or polystyrene) or glass

Second plate: 22 mm×25 mm size, 50 um thick plastic (as acrylic or polystyrene) with an array of pillar spacers on one side. The pillar spacers are 20×20 um in lateral size, and 20 um in height, and the inter-spacer distance is 150 um for the array.

Beads: 20 um in diameter beads with metal surface (as gold or silver) with an area concentration of 100/mm2 to 1000/mm2, which are uniformly pre-dried on the second plate.

Concentration area: on the surface of all the beads

### 3. Exemplary Embodiments of Beads-Enhanced Speedy Test (BEST)—Beads Based:

Another exemplary device comprises a first plate, a second plate, a pit array on the first plate, an array of spacers on the second plate, beads and concentration areas.

First plate: 24 mm×32 mm size, 1 mm thick plastic (as acrylic or polystyrene) or glass with pit array on one side. The pits are 12 um×12 um in lateral size, and 6 um in depth, and the inter-pit distance is 50 um.

Second plate: 22 mm×25 mm size, 175 um thick plastic (as acrylic or polystyrene) with an array of pillar spacers on one side. The pillar spacers are 20×20 um in lateral size, and 10 um in height, and the inter-spacer distance is 100 um.

Beads: 10 um in diameter beads with or without metal surface (gold or silver) with an area concentration of 100/mm2 to 1000/mm2, which are uniformly pre-dried on the first plate and mostly inside the pits.

Concentration area: on the surface of all the beads

### 4. Exemplary Embodiments of Beads-Enhanced Speedy Test (BEST)—Protrusions Based:

Another exemplary device comprises a first plate, a second plate, an array of first type of pillars (spacers) and an array of second type of pillars (protrusions) on the first plate, and concentration areas.

First plate: 24 mm×32 mm size, 1 mm thick plastic (as acrylic or polystyrene) or glass with the two pillar arrays on one side. The first type of pillars are 20×20 um in lateral size, and 10 um in height, and the inter-pillar distance is 150 um. The second type of pillars are 10 um in lateral diameter, and 8 um in height, and the inter-pillar distance is 50 um. The two pillar arrays are intermingled with one another.

Second plate: 22 mm×25 mm size, 150 um thick plastic (as acrylic or polystyrene) with flat surface.

Concentration area: on the top surface of the protrusions. Or on the side surfaces of the protrusions. Or on all the surfaces of the protrusions.

### 5. Exemplary Embodiments of Beads-Enhanced Speedy Test (BEST)—Protrusions Based:

Another exemplary device comprises a first plate, a second plate, an array of first type of pillars (spacers) and an array of second type of pillars (protrusions) on the first plate, and concentration areas.

First plate: 24 mm×32 mm size, 1 mm thick plastic (as acrylic or polystyrene) or glass with the two pillar arrays on one side. The first type pillars are 30×30 um in lateral size, and 15 um in height, and the inter-pillar distance is 120 um. The second type pillars are 15 um in lateral diameter, and 10 um in height, and the inter-pillar distance is 60 um. The two pillar arrays are intermingled with one another The second type pillars are coated with gold on all the surfaces.

Second plate: 22 mm×25 mm size, 100 um thick plastic (as acrylic or polystyrene) with flat surface.

Concentration area: on the top surface of the protrusions. Or on the side surfaces of the protrusions. Or on all the surfaces of the protrusions.

### 6. Exemplary Embodiments of Beads-Enhanced Speedy Test (BEST)—Protrusions Based:

Another exemplary device comprises a first plate, a second plate, an array of first type of pillars (protrusions) on the first plate, an array of second type of pillars (spacers) on the second plate, and concentration areas.

First plate: 24 mm×32 mm size, 1 mm thick plastic (as acrylic or polystyrene) or glass with the protrusion pillar array on one side. The protrusion pillars are 10 um pillar in lateral diameter, and 5 um in height, and the inter-pillar distance is 50 um.

Second plate: 22 mm×25 mm size, 50 um thick plastic (as acrylic or polystyrene) with flat surface. The spacer pillars are 20×20 um in lateral size, and 10 um in height, and the inter-pillar distance is 150 um.

Concentration area: on the top surface of the protrusions. Or on the side surfaces of the protrusions. Or on all the surfaces of the protrusions.

In all the above exemplary devices of this section, the side wall(s) of the protrusion pillars has/have a slope of 90%, 80%, 70%, 60%, 50%, 40%, 30%, 20%, or in a range between any of these two values.

### Machine Learning

Details of the Network are described in detail in a variety of publications including International Application (IA) No. PCT/US2018/017504 filed Feb. 8, 2018, and PCT/US2018/057877 filed Oct. 26, 2018, each of which are hereby incorporated by reference herein for all purposes.

One aspect of the present invention provides a framework of machine learning and deep learning for analyte detection and localization. A machine learning algorithm is an algorithm that is able to learn from data to detect, segment, and classify the analytes from the image of the sample. A more rigorous definition of machine learning is “A computer program is said to learn from experience E with respect to some class of tasks T and performance measure P, if its performance at tasks in T, as measured by P, improves with experience E.” It explores the algorithms that can earn from and make predictions on data—such algorithms overcome the static program instructions by making data driven predictions or decisions, through building a model from sample inputs.

Deep learning is a specific kind of machine learning based on a set of algorithms that attempt to model the high level abstractions in data. In a simple case, there might be two sets of neurons: ones that receive an input signal and ones that send an output signal. When the input layer receives an input, it passes on a modified version of the input to the next layer. In a deep network, there are many layers between the input and output (and the layers are not made of neurons but it can help to think of it that way), allowing the algorithm to use multiple processing layers, composed of multiple linear and non-linear transformations.

One aspect of the present invention is two machine learning based analyte detection and localization approaches. The first approach is a deep learning approach and the second approach is a combination of deep learning and computer vision approaches.

(i) Deep Learning Approach. In the first approach, the disclosed analyte detection and localization workflow consists of two stages, training and prediction. We describe training and prediction stages in the following paragraphs.

(a) Training Stage

In the training stage, training data with annotation is fed into a convolutional neural network. Convolutional neural network is a specialized neural network for processing data that has a grid-like, feed forward and layered network topology. Examples of the data include time-series data, which can be thought of as a 1D grid taking samples at regular time intervals, and image data, which can be thought of as a 2D grid of pixels. Convolutional networks have been successful in practical applications. The name “convolutional neural network” indicates that the network employs a mathematical operation called convolution. Convolution is a specialized kind of linear operation. Convolutional networks are simply neural networks that use convolution in place of general matrix multiplication in at least one of their layers.

The machine learning model receives one or multiple images of samples that contain the analytes taken by the imager over the sample holding QMAX device as training data. Training data are annotated for analytes to be assayed, wherein the annotations indicate whether or not analytes are in the training data and where they locate in the image. Annotation can be done in the form of tight bounding boxes which fully contains the analyte, or center locations of analytes. In the latter case, center locations are further converted into circles covering analytes or a Gaussian kernel in a point map.

When the size of training data is large, training machine learning model presents two challenges: annotation (usually done by human) is time consuming, and the training is computationally expensive. To overcome these challenges, one can partition the training data into patches of small size, then annotate and train on these patches, or a portion of these patches. The term “machine learning” refers to algorithms, systems and apparatus in the field of artificial intelligence that often use statistical techniques and artificial neural network trained from data without being explicitly programmed.

The annotated images are fed to the machine learning (ML) training module, and the model trainer in the machine learning module will train a ML model from the training data (annotated sample images). The input data will be fed to the model trainer in multiple iterations until certain stopping criterion is satisfied. The output of the ML training module is a ML model—a computational model that is built from a training process in the machine learning from the data that gives computer the capability to perform certain tasks (e.g. detect and classify the objects) on its own.

The trained machine learning model is applied during the predication (or inference) stage by the computer. Examples of machine learning models include ResNet, DenseNet, etc. which are also named as “deep learning models” because of the depth of the connected layers in their network structure. In certain embodiments, the Caffe library with fully convolutional network (FCN) was used for model training and predication, and other convolutional neural network architecture and library can also be used, such as TensorFlow.

The training stage generates a model that will be used in the prediction stage. The model can be repeatedly used in the prediction stage for assaying the input. Thus, the computing unit only needs access to the generated model. It does not need access to the training data, nor requiring the training stage to be run again on the computing unit.

(b) Prediction Stage

In the predication/inference stage, a detection component is applied to the input image, and an input image is fed into the predication (inference) module preloaded with a trained model generated from the training stage. The output of the prediction stage can be bounding boxes that contain the detected analytes with their center locations or a point map indicating the location of each analyte, or a heatmap that contains the information of the detected analytes.

When the output of the prediction stage is a list of bounding boxes, the number of analytes in the image of the sample for assaying is characterized by the number of detected bounding boxes. When the output of the prediction stage is a point map, the number of analytes in the image of the sample for assaying is characterized by the integration of the point map. When the output of the prediction is a heatmap, a localization component is used to identify the location, and from which, the number of detected analytes is characterized by the entries of the heatmap.

One embodiment of the localization algorithm is to sort the heatmap values into a one-dimensional ordered list, from the highest value to the lowest value. Then pick the pixel with the highest value, remove the pixel from the list, along with its neighbors. Iterate the process to pick the pixel with the highest value in the list, until all pixels are removed from the list.

In the detection component using heatmap, an input image, along with the model generated from the training stage, is fed into a convolutional neural network, and the output of the detection stage is a pixel-level prediction, in the form of a heatmap. The heatmap can have the same size as the input image, or it can be a scaled down version of the input image, and it is the input to the localization component. We disclose an algorithm to localize the analyte center. The main idea is to iteratively detect local peaks from the heatmap. After the peak is localized, we calculate the local area surrounding the peak but with smaller value. We remove this region from the heatmap and find the next peak from the remaining pixels. The process is repeated until all pixels are removed from the heatmap.

In certain embodiments, the present invention provides the localization algorithm to sort the heatmap values into a one-dimensional ordered list, from the highest value to the lowest value. Then pick the pixel with the highest value, remove the pixel from the list, along with its neighbors. Iterate the process to pick the pixel with the highest value in the list, until all pixels are removed from the list.

Algorithm GlobalSearch (heatmap)

```
Input:
  heatmap
Output:
  loci
loci ←{}
sort(heatmap)
while (heatmap is not empty) {
  s ← pop(heatmap)
  D ← {disk center as s with radius R}
  heatmap = heatmap \ D // remove D from the heatmap
  add s to loci
}
```

After sorting, heatmap is a one-dimensional ordered list, where the heatmap value is ordered from the highest to the lowest. Each heatmap value is associated with its corresponding pixel coordinates. The first item in the heatmap is the one with the highest value, which is the output of the pop (heatmap) function. One disk is created, where the center is the pixel coordinate of the one with highest heatmap value. Then all heatmap values whose pixel coordinates resides inside the disk is removed from the heatmap. The algorithm repeatedly pops up the highest value in the current heatmap, removes the disk around it, until all items are removed from the heatmap.

In the ordered list heatmap, each item has the knowledge of the proceeding item, and the following item. When removing an item from the ordered list, we make the following changes:
Assume the removing item is xᵣ, its proceeding item is xₚ, and its following item is x_{f}.
For the proceeding item xₚ, re-define its following item to the following item of the removing item. Thus, the following item of xₚ is now x_{f}.
For the removing item xᵣ, un-define its proceeding item and following item, which removes it from the ordered list.
For the following item x_{f}, re-define its proceeding item to the proceeding item of the removed item. Thus, the proceeding item of x_{f} is now xₚ.

After all items are removed from the ordered list, the localization algorithm is complete. The number of elements in the set loci will be the count of analytes, and location information is the pixel coordinate for each s in the set loci.

Another embodiment searches local peak, which is not necessary the one with the highest heatmap value. To detect each local peak, we start from a random starting point, and search for the local maximal value. After we find the local peak, we calculate the local area surrounding the peak but with smaller value. We remove this region from the heatmap and find the next peak from the remaining pixels. The process is repeated only all pixels are removed from the heatmap.

```
Algorithm LocalSearch (s, heatmap)
Input:
  s: starting location (x, y)
  heatmap
Output:
  s: location of local peak.
We only consider pixels of value > 0.
Algorithm Cover (s, heatmap)
Input:
  s: location of local peak.
  heatmap:
Output:
  cover: a set of pixels covered by peak:
```

This is a breadth-first-search algorithm starting from s, with one altered condition of visiting points: a neighbor p of the current location q is only added to cover if heatmap[p]>0 and heatmap[p]<=heatmap[q]. Therefore, each pixel in cover has a non-descending path leading to the local peak s.

(ii) Mixture of Deep Learning and Computer Vision Approach. In the second approach, the detection and localization are realized by computer vision algorithms, and the classification is realized by deep learning algorithms, wherein the computer vision algorithms detect and locate possible candidates of analytes, and the deep learning algorithm classifies each possible candidate as a true analyte and false analyte. The location of all true analyte (along with the total count of true analytes) will be recorded as the output.

(a) Detection. The computer vision algorithm detects possible candidate based on the characteristics of analytes, including but not limited to intensity, color, size, shape, distribution, etc. A pre-processing scheme can improve the detection. Pre-processing schemes include contrast enhancement, histogram adjustment, color enhancement, de-nosing, smoothing, de-focus, etc. After pre-processing, the input image is sent to a detector. The detector tells the existing of possible candidate of analyte and gives an estimate of its location. The detection can be based on the analyte structure (such as edge detection, line detection, circle detection, etc.), the connectivity (such as blob detection, connect components, contour detection, etc.), intensity, color, shape using schemes such as adaptive thresholding, etc.

(b) Localization. After detection, the computer vision algorithm locates each possible candidate of analytes by providing its boundary or a tight bounding box containing it. This can be achieved through object segmentation algorithms, such as adaptive thresholding, background subtraction, floodfill, mean shift, watershed, etc. Very often, the localization can be combined with detection to produce the detection results along with the location of each possible candidates of analytes.

(c) Classification. The deep learning algorithms, such as convolutional neural networks, achieve start-of-the-art visual classification. We employ deep learning algorithms for classification on each possible candidate of analytes. Various convolutional neural network can be utilized for analyte classification, such as VGGNet, ResNet, MobileNet, DenseNet, etc.

Given each possible candidate of analyte, the deep learning algorithm computes through layers of neurons via convolution filters and non-linear filters to extract high-level features that differentiate analyte against non-analytes. A layer of fully convolutional network will combine high-level features into classification results, which tells whether it is a true analyte or not, or the probability of being a analyte.

Moreover, for people skilled in the field, these two approaches can be further extended and mixed. A mixture of deep learning and computer vision can become even more deep learning oriented by applying computer vision algorithms only for pre-processing of the image, whereas each step in detection, localization, and classification is based on the dedicated deep learning model or using one deep learning model, such as RetinaNet, for doing one step detection and classification.

### Applications, Bio/Chemical Biomarkers, and Health Conditions

The applications of the present invention include, but not limited to, (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g.,

The detection can be carried out in various sample matrix, such as cells, tissues, bodily fluids, and stool. Bodily fluids of interest include but are not limited to, amniotic fluid, aqueous humour, vitreous humour, blood (e.g., whole blood, fractionated blood, plasma, serum, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine and exhaled condensate. In some embodiments, the sample comprises a human body fluid. In some embodiments, the sample comprises at least one of cells, tissues, bodily fluids, stool, amniotic fluid, aqueous humour, vitreous humour, blood, whole blood, fractionated blood, plasma, serum, breast milk, cerebrospinal fluid, cerumen, chyle, chime, endolymph, perilymph, feces, gastric acid, gastric juice, lymph, mucus, nasal drainage, phlegm, pericardial fluid, peritoneal fluid, pleural fluid, pus, rheum, saliva, sebum, semen, sputum, sweat, synovial fluid, tears, vomit, urine, and exhaled condensate.

In embodiments, the sample is at least one of a biological sample, an environmental sample, and a biochemical sample.

The devices, systems and the methods in the present invention find use in a variety of different applications in various fields, where determination of the presence or absence, and/or quantification of one or more analytes in a sample are desired. For example, the subject method finds use in the detection of proteins, peptides, nucleic acids, synthetic compounds, inorganic compounds, and the like. The various fields include, but not limited to, human, veterinary, agriculture, foods, environments, drug testing, and others.

In certain embodiments, the subject method finds use in the detection of nucleic acids, proteins, or other biomolecules in a sample. The methods can include the detection of a set of biomarkers, e.g., two or more distinct protein or nucleic acid biomarkers, in a sample. For example, the methods can be used in the rapid, clinical detection of two or more disease biomarkers in a biological sample, e.g., as can be employed in the diagnosis of a disease condition in a subject, or in the ongoing management or treatment of a disease condition in a subject, etc. As described above, communication to a physician or other health-care provider can better ensure that the physician or other health-care provider is made aware of, and cognizant of, possible concerns and can thus be more likely to take appropriate action.

The applications of the devices, systems and methods in the present inventions of employing a CROF device include, but are not limited to, (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals. Some of the specific applications of the devices, systems and methods in the present invention are described now in further detail.

The applications of the present invention include, but not limited to, (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals.

An implementation of the devices, systems and methods in the present invention can include a) obtaining a sample, b) applying the sample to CROF device containing a capture agent that binds to an analyte of interest, under conditions suitable for binding of the analyte in a sample to the capture agent, c) washing the CROF device, and d) reading the CROF device, thereby obtaining a measurement of the amount of the analyte in the sample. In some embodiments, the analyte can be a biomarker, an environmental marker, or a foodstuff marker. The sample in some instances is a liquid sample, and can be a diagnostic sample (such as saliva, serum, blood, sputum, urine, sweat, lacrima, semen, or mucus); an environmental sample obtained from a river, ocean, lake, rain, snow, sewage, sewage processing runoff, agricultural runoff, industrial runoff, tap water or drinking water; or a foodstuff sample obtained from tap water, drinking water, prepared food, processed food or raw food.

In any embodiment, the CROF device can be placed in a microfluidic device and the applying step b) can include applying a sample to a microfluidic device comprising the CROF device.

In any embodiment, the reading step d) can include detecting a fluorescence or luminescence signal from the CROF device.

In any embodiment, the reading step d) can include reading the CROF device with a handheld device configured to read the CROF device. The handheld device can be a mobile phone, e.g., a smart phone.

In any embodiment, the CROF device can include a labeling agent that can bind to an analyte-capture agent complex on the CROF device.

In any embodiment, the devices, systems and methods in the present invention can further include, between steps c) and d), the steps of applying to the CROF device a labeling agent that binds to an analyte-capture agent complex on the CROF device, and washing the CROF device.

In any embodiment, the reading step d) can include reading an identifier for the CROF device. The identifier can be an optical barcode, a radio frequency ID tag, or combinations thereof.

In any embodiment, the devices, systems and methods in the present invention can further include applying a control sample to a control CROF device containing a capture agent that binds to the analyte, wherein the control sample includes a known detectable amount of the analyte, and reading the control CROF device, thereby obtaining a control measurement for the known detectable amount of the analyte in a sample.

In any embodiment, the sample can be a diagnostic sample obtained from a subject, the analyte can be a biomarker, and the measured amount of the analyte in the sample can be diagnostic of a disease or a condition.

In any embodiment, the devices, systems and methods in the present invention can further include receiving or providing to the subject a report that indicates the measured amount of the biomarker and a range of measured values for the biomarker in an individual free of or at low risk of having the disease or condition, wherein the measured amount of the biomarker relative to the range of measured values is diagnostic of a disease or condition.

In any embodiment, the devices, systems and methods in the present invention can further include diagnosing the subject based on information including the measured amount of the biomarker in the sample. In some cases, the diagnosing step includes sending data containing the measured amount of the biomarker to a remote location and receiving a diagnosis based on information including the measurement from the remote location.

In any embodiment, the applying step b) can include isolating miRNA from the sample to generate an isolated miRNA sample, and applying the isolated miRNA sample to the disk-coupled dots-on-pillar antenna (CROF device) array.

In any embodiment, the method can include receiving or providing a report that indicates the safety or harmfulness for a subject to be exposed to the environment from which the sample was obtained.

In any embodiment, the method can include sending data containing the measured amount of the environmental marker to a remote location and receiving a report that indicates the safety or harmfulness for a subject to be exposed to the environment from which the sample was obtained.

In any embodiment, the CROF device array can include a plurality of capture agents that each binds to an environmental marker, and wherein the reading step d) can include obtaining a measure of the amount of the plurality of environmental markers in the sample.

In any embodiment, the sample can be a foodstuff sample, wherein the analyte can be a foodstuff marker, and wherein the amount of the foodstuff marker in the sample can correlate with safety of the foodstuff for consumption.

In any embodiment, the method can include receiving or providing a report that indicates the safety or harmfulness for a subject to consume the foodstuff from which the sample is obtained.

In any embodiment, the method can include sending data containing the measured amount of the foodstuff marker to a remote location and receiving a report that indicates the safety or harmfulness for a subject to consume the foodstuff from which the sample is obtained.

In any embodiment, the CROF device array can include a plurality of capture agents that each binds to a foodstuff marker, wherein the obtaining can include obtaining a measure of the amount of the plurality of foodstuff markers in the sample, and wherein the amount of the plurality of foodstuff marker in the sample can correlate with safety of the foodstuff for consumption.

Also provided herein are kits that find use in practicing the devices, systems and methods in the present invention.

The amount of sample can be about a drop of a sample. The amount of sample can be the amount collected from a pricked finger or fingerstick. The amount of sample can be the amount collected from a microneedle or a venous draw.

A sample can be used without further processing after obtaining it from the source, or can be processed, e.g., to enrich for an analyte of interest, remove large particulate matter, dissolve or resuspend a solid sample, etc.

Any suitable method of applying a sample to the CROF device can be employed. Suitable methods can include using a pipet, dropper, syringe, etc. In certain embodiments, when the CROF device is located on a support in a dipstick format, as described below, the sample can be applied to the CROF device by dipping a sample-receiving area of the dipstick into the sample.

A sample can be collected at one time, or at a plurality of times. Samples collected over time can be aggregated and/or processed (by applying to a CROF device and obtaining a measurement of the amount of analyte in the sample, as described herein) individually. In some instances, measurements obtained over time can be aggregated and can be useful for longitudinal analysis over time to facilitate screening, diagnosis, treatment, and/or disease prevention.

Washing the CROF device to remove unbound sample components can be done in any convenient manner, as described above. In certain embodiments, the surface of the CROF device is washed using binding buffer to remove unbound sample components.

Detectable labeling of the analyte can be done by any convenient method. The analyte can be labeled directly or indirectly. In direct labeling, the analyte in the sample is labeled before the sample is applied to the CROF device. In indirect labeling, an unlabeled analyte in a sample is labeled after the sample is applied to the CROF device to capture the unlabeled analyte, as described below.

The samples from a subject, the health of a subject, and other applications of the present invention are further described below. Exemplary samples, health conditions, and application are also disclosed in, e.g., U.S. Pub. Nos. 2014/0154668 and 2014/0045209, which are hereby incorporated by reference.

The present inventions find use in a variety of applications, where such applications are generally analyte detection applications in which the presence of a particular analyte in a given sample is detected at least qualitatively, if not quantitatively. Protocols for carrying out analyte detection assays are well known to those of skill in the art and need not be described in great detail here. Generally, the sample suspected of comprising an analyte of interest is contacted with the surface of a subject nanosensor under conditions sufficient for the analyte to bind to its respective capture agent that is tethered to the sensor. The capture agent has highly specific affinity for the targeted molecules of interest. This affinity can be antigen-antibody reaction where antibodies bind to specific epitope on the antigen, or a DNA/RNA or DNA/RNA hybridization reaction that is sequence-specific between two or more complementary strands of nucleic acids. Thus, if the analyte of interest is present in the sample, it likely binds to the sensor at the site of the capture agent and a complex is formed on the sensor surface. Namely, the captured analytes are immobilized at the sensor surface. After removing the unbounded analytes, the presence of this binding complex on the surface of the sensor (e.g., the immobilized analytes of interest) is then detected, e.g., using a labeled secondary capture agent.

Specific analyte detection applications of interest include hybridization assays in which the nucleic acid capture agents are employed and protein binding assays in which polypeptides, e.g., antibodies, are employed. In these assays, a sample is first prepared and following sample preparation, the sample is contacted with a subject nanosensor under specific binding conditions, whereby complexes are formed between target nucleic acids or polypeptides (or other molecules) that are complementary to capture agents attached to the sensor surface.

In one embodiment, the capture oligonucleotide is synthesized single strand DNA of 20-100 bases length, that is thiolated at one end. These molecules are immobilized on the nanodevices' surface to capture the targeted single-strand DNA (which can be at least 50 bp length) that has a sequence that is complementary to the immobilized capture DNA. After the hybridization reaction, a detection single strand DNA (which can be of 20-100 bp in length) whose sequence are complementary to the targeted DNA's unoccupied nucleic acid is added to hybridize with the target. The detection DNA has its one end conjugated to a fluorescence label, whose emission wavelength are within the plasmonic resonance of the nanodevice. Therefore by detecting the fluorescence emission emanate from the nanodevices' surface, the targeted single strand DNA can be accurately detected and quantified. The length for capture and detection DNA determine the melting temperature (nucleotide strands will separate above melting temperature), the extent of misparing (the longer the strand, the lower the misparing).

One of the concerns of choosing the length for complementary binding depends on the needs to minimize misparing while keeping the melting temperature as high as possible. In addition, the total length of the hybridization length is determined in order to achieve optimum signal amplification.

A subject sensor can be employed in a method of diagnosing a disease or condition, comprising: (a) obtaining a liquid sample from a patient suspected of having the disease or condition, (b) contacting the sample with a subject nanosensor, wherein the capture agent of the nanosensor specifically binds to a biomarker for the disease and wherein the contacting is done under conditions suitable for specific binding of the biomarker with the capture agent; (c) removing any biomarker that is not bound to the capture agent; and (d) reading a light signal from biomarker that remain bound to the nanosensor, wherein a light signal indicates that the patient has the disease or condition, wherein the method further comprises labeling the biomarker with a light-emitting label, either prior to or after it is bound to the capture agent. As will be described in greater detail below, the patient can suspected of having cancer and the antibody binds to a cancer biomarker. In other embodiments, the patient is suspected of having a neurological disorder and the antibody binds to a biomarker for the neurological disorder. The applications of the subject sensor include, but not limited to, (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals.

The detection can be carried out in various sample matrix, such as cells, tissues, bodily fluids, and stool. Bodily fluids of interest include but are not limited to, amniotic fluid, aqueous humour, vitreous humour, blood (e.g., whole blood, fractionated blood, plasma, serum, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine and exhaled condensate.

In some embodiments, a subject biosensor can be used diagnose a pathogen infection by detecting a target nucleic acid from a pathogen in a sample. The target nucleic acid can be, for example, from a virus that is selected from the group comprising human immunodeficiency virus 1 and 2 (HIV-1 and HIV-2), human T-cell leukaemia virus and 2 (HTLV-1 and HTLV-2), respiratory syncytial virus (RSV), adenovirus, hepatitis B virus (HBV), hepatitis C virus (HCV), Epstein-Barr virus (EBV), human papillomavirus (HPV), varicella zoster virus (VZV), cytomegalovirus (CMV), herpes-simplex virus 1 and 2 (HSV-1 and HSV-2), human herpesvirus 8 (HHV-8, also known as Kaposi sarcoma herpesvirus) and flaviviruses, including yellow fever virus, dengue virus, Japanese encephalitis virus, West Nile virus and Ebola virus. The present invention is not, however, limited to the detection of nucleic acid, e.g., DNA or RNA, sequences from the aforementioned viruses, but can be applied without any problem to other pathogens important in veterinary and/or human medicine.

Human papillomaviruses (HPV) are further subdivided on the basis of their DNA sequence homology into more than 70 different types. These types cause different diseases. HPV types 1, 2, 3, 4, 7, 10 and 26-29 cause benign warts. HPV types 5, 8, 9, 12, 14, 15, 17 and 19-25 and 46-50 cause lesions in patients with a weakened immune system. Types 6, 11, 34, 39, 41-44 and 51-55 cause benign acuminate warts on the mucosae of the genital region and of the respiratory tract. HPV types 16 and 18 are of special medical interest, as they cause epithelial dysplasias of the genital mucosa and are associated with a high proportion of the invasive carcinomas of the cervix, vagina, vulva and anal canal. Integration of the DNA of the human papillomavirus is considered to be decisive in the carcinogenesis of cervical cancer. Human papillomaviruses can be detected for example from the DNA sequence of their capsid proteins L1 and L2. Accordingly, the method of the present invention is especially suitable for the detection of DNA sequences of HPV types 16 and/or 18 in tissue samples, for assessing the risk of development of carcinoma.

In some cases, the nanosensor can be employed to detect a biomarker that is present at a low concentration. For example, the nanosensor can be used to detect cancer antigens in a readily accessible bodily fluids (e.g., blood, saliva, urine, tears, etc.), to detect biomarkers for tissue-specific diseases in a readily accessible bodily fluid (e.g., a biomarkers for a neurological disorder (e.g., Alzheimer's antigens)), to detect infections (particularly detection of low titer latent viruses, e.g., HIV), to detect fetal antigens in maternal blood, and for detection of exogenous compounds (e.g., drugs or pollutants) in a subject's bloodstream, for example. The following table provides a list of protein biomarkers that can be detected using the subject nanosensor (when used in conjunction with an appropriate monoclonal antibody), and their associated diseases. One potential source of the biomarker (e.g., “CSF”; cerebrospinal fluid) is also indicated in the table. In many cases, the subject biosensor can detect those biomarkers in a different bodily fluid to that indicated. For example, biomarkers that are found in CSF can be identified in urine, blood or saliva.

A) Utility

The subject method finds use in a variety of different applications where determination of the presence or absence, and/or quantification of one or more analytes in a sample are desired. For example, the subject method finds use in the detection of proteins, peptides, nucleic acids, synthetic compounds, inorganic compounds, and the like.

In certain embodiments, the subject method finds use in the detection of nucleic acids, proteins, or other biomolecules in a sample. The methods can include the detection of a set of biomarkers, e.g., two or more distinct protein or nucleic acid biomarkers, in a sample. For example, the methods can be used in the rapid, clinical detection of two or more disease biomarkers in a biological sample, e.g., as can be employed in the diagnosis of a disease condition in a subject, or in the ongoing management or treatment of a disease condition in a subject, etc. As described above, communication to a physician or other health-care provider can better ensure that the physician or other health-care provider is made aware of, and cognizant of, possible concerns and can thus be more likely to take appropriate action.

The applications of the devices, systems and methods in the present invention of employing a CROF device include, but are not limited to, (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals. Some of the specific applications of the devices, systems and methods in the present invention are described now in further detail.

B) Diagnostic Method

In certain embodiments, the subject method finds use in detecting biomarkers. In some embodiments, the devices, systems and methods in the present invention of using CROF are used to detect the presence or absence of particular biomarkers, as well as an increase or decrease in the concentration of particular biomarkers in blood, plasma, serum, or other bodily fluids or excretions, such as but not limited to urine, blood, serum, plasma, saliva, semen, prostatic fluid, nipple aspirate fluid, lachrymal fluid, perspiration, feces, cheek swabs, cerebrospinal fluid, cell lysate samples, amniotic fluid, gastrointestinal fluid, biopsy tissue, and the like. Thus, the sample, e.g. a diagnostic sample, can include various fluid or solid samples.

In some instances, the sample can be a bodily fluid sample from a subject who is to be diagnosed. In some instances, solid or semi-solid samples can be provided. The sample can include tissues and/or cells collected from the subject. The sample can be a biological sample. Examples of biological samples can include but are not limited to, blood, serum, plasma, a nasal swab, a nasopharyngeal wash, saliva, urine, gastric fluid, spinal fluid, tears, stool, mucus, sweat, earwax, oil, a glandular secretion, cerebral spinal fluid, tissue, semen, vaginal fluid, interstitial fluids derived from tumorous tissue, ocular fluids, spinal fluid, a throat swab, breath, hair, finger nails, skin, biopsy, placental fluid, amniotic fluid, cord blood, lymphatic fluids, cavity fluids, sputum, pus, microbiota, meconium, breast milk, exhaled condensate and/or other excretions. The samples can include nasopharyngeal wash. Nasal swabs, throat swabs, stool samples, hair, finger nail, ear wax, breath, and other solid, semi-solid, or gaseous samples can be processed in an extraction buffer, e.g., for a fixed or variable amount of time, prior to their analysis. The extraction buffer or an aliquot thereof can then be processed similarly to other fluid samples if desired. Examples of tissue samples of the subject can include but are not limited to, connective tissue, muscle tissue, nervous tissue, epithelial tissue, cartilage, cancerous sample, or bone.

In some instances, the subject from which a diagnostic sample is obtained can be a healthy individual, or can be an individual at least suspected of having a disease or a health condition. In some instances, the subject can be a patient.

In certain embodiments, the CROF device includes a capture agent configured to specifically bind a biomarker in a sample provided by the subject. In certain embodiments, the biomarker can be a protein. In certain embodiments, the biomarker protein is specifically bound by an antibody capture agent present in the CROF device. In certain embodiments, the biomarker is an antibody specifically bound by an antigen capture agent present in the CROF device. In certain embodiments, the biomarker is a nucleic acid specifically bound by a nucleic acid capture agent that is complementary to one or both strands of a double-stranded nucleic acid biomarker, or complementary to a single-stranded biomarker. In certain embodiments, the biomarker is a nucleic acid specifically bound by a nucleic acid binding protein. In certain embodiments, the biomarker is specifically bound by an aptamer.

The presence or absence of a biomarker or significant changes in the concentration of a biomarker can be used to diagnose disease risk, presence of disease in an individual, or to tailor treatments for the disease in an individual. For example, the presence of a particular biomarker or panel of biomarkers can influence the choices of drug treatment or administration regimes given to an individual. In evaluating potential drug therapies, a biomarker can be used as a surrogate for a natural endpoint such as survival or irreversible morbidity. If a treatment alters the biomarker, which has a direct connection to improved health, the biomarker can serve as a surrogate endpoint for evaluating the clinical benefit of a particular treatment or administration regime. Thus, personalized diagnosis and treatment based on the particular biomarkers or panel of biomarkers detected in an individual are facilitated by the subject method. Furthermore, the early detection of biomarkers associated with diseases is facilitated by the high sensitivity of the devices, systems and methods in the present invention, as described above. Due to the capability of detecting multiple biomarkers with a mobile device, such as a smartphone, combined with sensitivity, scalability, and ease of use, the presently disclosed method finds use in portable and point-of-care or near-patient molecular diagnostics.

In certain embodiments, the subject method finds use in detecting biomarkers for a disease or disease state. In certain instances, the subject method finds use in detecting biomarkers for the characterization of cell signaling pathways and intracellular communication for drug discovery and vaccine development. For example, the subject method can be used to detect and/or quantify the amount of biomarkers in diseased, healthy or benign samples. In certain embodiments, the subject method finds use in detecting biomarkers for an infectious disease or disease state. In some cases, the biomarkers can be molecular biomarkers, such as but not limited to proteins, nucleic acids, carbohydrates, small molecules, and the like.

The subject method find use in diagnostic assays, such as, but not limited to, the following: detecting and/or quantifying biomarkers, as described above; screening assays, where samples are tested at regular intervals for asymptomatic subjects; prognostic assays, where the presence and or quantity of a biomarker is used to predict a likely disease course; stratification assays, where a subject's response to different drug treatments can be predicted; efficacy assays, where the efficacy of a drug treatment is monitored; and the like.

In some embodiments, a subject biosensor can be used diagnose a pathogen infection by detecting a target nucleic acid from a pathogen in a sample. The target nucleic acid can be, for example, from a virus that is selected from the group comprising human immunodeficiency virus 1 and 2 (HIV-1 and HIV-2), human T-cell leukaemia virus and 2 (HTLV-1 and HTLV-2), respiratory syncytial virus (RSV), adenovirus, hepatitis B virus (HBV), hepatitis C virus (HCV), Epstein-Barr virus (EBV), human papillomavirus (HPV), varicella zoster virus (VZV), cytomegalovirus (CMV), herpes-simplex virus 1 and 2 (HSV-1 and HSV-2), human herpesvirus 8 (HHV-8, also known as Kaposi sarcoma herpesvirus) and flaviviruses, including yellow fever virus, dengue virus, Japanese encephalitis virus, West Nile virus and Ebola virus. The present invention is not, however, limited to the detection of nucleic acid, e.g., DNA or RNA, sequences from the aforementioned viruses, but can be applied without any problem to other pathogens important in veterinary and/or human medicine.

Human papillomaviruses (HPV) are further subdivided on the basis of their DNA sequence homology into more than 70 different types. These types cause different diseases. HPV types 1, 2, 3, 4, 7, 10 and 26-29 cause benign warts. HPV types 5, 8, 9, 12, 14, 15, 17 and 19-25 and 46-50 cause lesions in patients with a weakened immune system. Types 6, 11, 34, 39, 41-44 and 51-55 cause benign acuminate warts on the mucosae of the genital region and of the respiratory tract. HPV types 16 and 18 are of special medical interest, as they cause epithelial dysplasias of the genital mucosa and are associated with a high proportion of the invasive carcinomas of the cervix, vagina, vulva and anal canal. Integration of the DNA of the human papillomavirus is considered to be decisive in the carcinogenesis of cervical cancer. Human papillomaviruses can be detected for example from the DNA sequence of their capsid proteins L1 and L2. Accordingly, the method of the present invention is especially suitable for the detection of DNA sequences of HPV types 16 and/or 18 in tissue samples, for assessing the risk of development of carcinoma.

Other pathogens that can be detected in a diagnostic sample using the devices, systems and methods in the present invention include, but are not limited to: Varicella zoster Staphylococcus epidermidis, Escherichia coli, methicillin-resistant Staphylococcus aureus (MSRA), Staphylococcus aureus, Staphylococcus hominis, Enterococcus faecalis, Pseudomonas aeruginosa, Staphylococcus capitis, Staphylococcus wameri, Klebsiella pneumoniae, Haemophilus influenzae, Staphylococcus simulans, Streptococcus pneumoniae and Candida albicans; gonorrhea (Neisseria gonorrhoeae), syphilis (Treponena pallidum), Chlamydia (Chlamydia tracomitis), nongonococcal urethritis (Ureaplasm urealyticum), chancroid (Haemophilus ducreyi), trichomoniasis (Trichomonas vaginalis); Pseudomonas aeruginosa, methicillin-resistant Staphylococcus aureus (MSRA), Klebsiella pneumoniae, Haemophilus influenzae, Staphylococcus aureus, Stenotrophomonas maltophilia, Haemophilus parainfluenzae, Escherichia coli, Enterococcus faecalis, Serratia marcescens, Haemophilus parahaemolyticus, Enterococcus cloacae, Candida albicans, Moraxella catarrhalis, Streptococcus pneumoniae, Citrobacter freundii, Enterococcus faecium, Klebsiella oxytoca, Pseudomonas fluorescens, Neisseria meningitidis, Streptococcus pyogenes, Pneumocystis carinii, Klebsiella pneumoniae Legionella pneumophila, Mycoplasma pneumoniae, and Mycobacterium tuberculosis, etc.

In some cases, the CROF device can be employed to detect a biomarker that is present at a low concentration. For example, the CROF device can be used to detect cancer antigens in a readily accessible bodily fluids (e.g., blood, saliva, urine, tears, etc.), to detect biomarkers for tissue-specific diseases in a readily accessible bodily fluid (e.g., a biomarkers for a neurological disorder (e.g., Alzheimer's antigens)), to detect infections (particularly detection of low titer latent viruses, e.g., HIV), to detect fetal antigens in maternal blood, and for detection of exogenous compounds (e.g., drugs or pollutants) in a subject's bloodstream, for example.

One potential source of the biomarker (e.g., “CSF”; cerebrospinal fluid) is also indicated in the table. In many cases, the subject biosensor can detect those biomarkers in a different bodily fluid to that indicated. For example, biomarkers that are found in CSF can be identified in urine, blood or saliva. It will also be clear to one with ordinary skill in the art that the subject CROF devices can be configured to capture and detect many more biomarkers known in the art that are diagnostic of a disease or health condition.

A biomarker can be a protein or a nucleic acid (e.g., mRNA) biomarker, unless specified otherwise. The diagnosis can be associated with an increase or a decrease in the level of a biomarker in the sample, unless specified otherwise. Lists of biomarkers, the diseases that they can be used to diagnose, and the sample in which the biomarkers can be detected are described in Tables 1 and 2 of U.S. provisional application Ser. No. 62/234,538, filed on Sep. 29, 2015, which application is incorporated by reference herein.

In some instances, the devices, systems and methods in the present invention is used to inform the subject from whom the sample is derived about a health condition thereof. Health conditions that can be diagnosed or measured by the devices, systems and methods in the present invention, device and system include, but are not limited to: chemical balance; nutritional health; exercise; fatigue; sleep; stress; prediabetes; allergies; aging; exposure to environmental toxins, pesticides, herbicides, synthetic hormone analogs; pregnancy; menopause; and andropause. Table 3 of U.S. provisional application Ser. No. 62/234,538, filed on Sep. 29, 2015, which application is incorporated by reference herein, provides a list of biomarker that can be detected using the present CROF device (when used in conjunction with an appropriate monoclonal antibody, nucleic acid, or other capture agent), and their associated health conditions.

C) Kits

Aspects of the present disclosure include a kit that find use in performing the devices, systems and methods in the present invention, as described above. In certain embodiments, the kit includes instructions for practicing the subject methods using a hand held device, e.g., a mobile phone. These instructions can be present in the subject kits in a variety of forms, one or more of which can be present in the kit. One form in which these instructions can be present is as printed information on a suitable medium or substrate, e.g., a piece or pieces of paper on which the information is printed, in the packaging of the kit, in a package insert, etc. Another means would be a computer readable medium, e.g., diskette, CD, DVD, Blu-Ray, computer-readable memory, etc., on which the information has been recorded or stored. Yet another means that can be present is a website address which can be used via the Internet to access the information at a removed site. The kit can further include a software for implementing a method for measuring an analyte on a device, as described herein, provided on a computer readable medium. Any convenient means can be present in the kits.

In some embodiments, the kit includes a detection agent that includes a detectable label, e.g. a fluorescently labeled antibody or oligonucleotide that binds specifically to an analyte of interest, for use in labeling the analyte of interest. The detection agent can be provided in a separate container as the CROF device, or can be provided in the CROF device.

In some embodiments, the kit includes a control sample that includes a known detectable amount of an analyte that is to be detected in the sample. The control sample can be provided in a container, and can be in solution at a known concentration, or can be provided in dry form, e.g., lyophilized or freeze dried. The kit can also include buffers for use in dissolving the control sample, if it is provided in dry form.

### Related Documents

The present invention includes a variety of embodiments, which can be combined in multiple ways as long as the various components do not contradict one another. The embodiments should be regarded as a single invention file: each filing has other filing as the references and is also referenced in its entirety and for all purpose, rather than as a discrete independent. These embodiments include not only the disclosures in the current file, but also the documents that are herein referenced, incorporated, or to which priority is claimed.

### (1) Definitions

The terms used in describing the devices, systems, and methods herein disclosed are defined in the current application, or in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

The terms “CROF Card (or card)”, “COF Card”, “QMAX-Card”, “Q-Card”, “CROF device”, “COF device”, “QMAX-device”, “CROF plates”, “COF plates”, and “QMAX-plates” are interchangeable, except that in some embodiments, the COF card does not comprise spacers; and the terms refer to a device that comprises a first plate and a second plate that are movable relative to each other into different configurations (including an open configuration and a closed configuration), and that comprises spacers (except some embodiments of the COF card) that regulate the spacing between the plates. The term “X-plate” refers to one of the two plates in a CROF card, wherein the spacers are fixed to this plate. More descriptions of the COF Card, CROF Card, and X-plate are given in the provisional application Ser. No. 62/456,065, filed on Feb. 7, 2017, which is incorporated herein in its entirety for all purposes.

### (2) Q-Card, Spacer and Uniform Sample Thickness

The devices, systems, and methods herein disclosed can include or use Q-cards, spacers, and uniform sample thickness embodiments for sample detection, analysis, and quantification. In some embodiments, the Q-card comprises spacers, which help to render at least part of the sample into a layer of high uniformity. The structure, material, function, variation and dimension of the spacers, as well as the uniformity of the spacers and the sample layer, are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

### (3) Hinges, Opening Notches, Recessed Edge and Sliders

The devices, systems, and methods herein disclosed can include or use Q-cards for sample detection, analysis, and quantification. In some embodiments, the Q-card comprises hinges, notches, recesses, and sliders, which help to facilitate the manipulation of the Q card and the measurement of the samples. The structure, material, function, variation and dimension of the hinges, notches, recesses, and sliders are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

### (4) Q-Card, Sliders, and Smartphone Detection System

The Devices, Systems, and Methods Herein Disclosed can Include or Use Q-Cards for sample detection, analysis, and quantification. In some embodiments, the Q-cards are used together with sliders that allow the card to be read by a smartphone detection system. The structure, material, function, variation, dimension and connection of the Q-card, the sliders, and the smartphone detection system are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

### (5) Detection Methods

The devices, systems, and methods herein disclosed can include or be used in various types of detection methods. The detection methods are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

### (6) Labels, Capture Agent and Detection Agent

The devices, systems, and methods herein disclosed can employ various types of labels, capture agents, and detection agents that are used for analytes detection. The labels are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

### (7) Analytes

The devices, systems, and methods herein disclosed can be applied to manipulation and detection of various types of analytes (including biomarkers). The analytes and are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

### (8) Applications (Field and Samples)

The devices, systems, and methods herein disclosed can be used for various applications (fields and samples). The applications are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

### (9) Cloud

The devices, systems, and methods herein disclosed can employ cloud technology for data transfer, storage, and/or analysis. The related cloud technologies are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

### Other Embodiments

Further examples of inventive subject matter according to the present disclosure are described in the following enumerated paragraphs.

It must be noted that, as used herein and in the appended claims, the singular forms “a”, “an”, and “the” include plural referents unless the context clearly dictates otherwise, e.g., when the word “single” is used. For example, reference to “an analyte” includes a single analyte and multiple analytes, reference to “a capture agent” includes a single capture agent and multiple capture agents, reference to “a detection agent” includes a single detection agent and multiple detection agents, and reference to “an agent” includes a single agent and multiple agents.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. The term “about” or “approximately” can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed. The term “about” has the meaning as commonly understood by one of ordinary skill in the art. In some embodiments, the term “about” refers to ±10%. In some embodiments, the term “about” refers to ±5%.

As used herein, the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function. Similarly, subject matter that is recited as being configured to perform a particular function can additionally or alternatively be described as being operative to perform that function.

As used herein, the phrase, “for example,” the phrase, “as an example,” and/or simply the terms “example” and “exemplary” when used with reference to one or more components, features, details, structures, embodiments, and/or methods according to the present disclosure, are intended to convey that the described component, feature, detail, structure, embodiment, and/or method is an illustrative, non-exclusive example of components, features, details, structures, embodiments, and/or methods according to the present disclosure. Thus, the described component, feature, detail, structure, embodiment, and/or method is not intended to be limiting, required, or exclusive/exhaustive; and other components, features, details, structures, embodiments, and/or methods, including structurally and/or functionally similar and/or equivalent components, features, details, structures, embodiments, and/or methods, are also within the scope of the present disclosure.

As used herein, the phrases “at least one of” and “one or more of,” in reference to a list of more than one entity, means any one or more of the entity in the list of entity, and is not limited to at least one of each and every entity specifically listed within the list of entity. For example, “at least one of A and B” (or, equivalently, “at least one of A or B,” or, equivalently, “at least one of A and/or B”) can refer to A alone, B alone, or the combination of A and B.

As used herein, the term “and/or” placed between a first entity and a second entity means one of (1) the first entity, (2) the second entity, and (3) the first entity and the second entity. Multiple entity listed with “and/or” should be construed in the same manner, e.g., “one or more” of the entity so conjoined. Other entity can optionally be present other than the entity specifically identified by the “and/or” clause, whether related or unrelated to those entities specifically identified.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art.

In the event that any patents, patent applications, or other references are incorporated by reference herein and (1) define a term in a manner that is inconsistent with and/or (2) are otherwise inconsistent with, either the non-incorporated portion of the present disclosure or any of the other incorporated references, the non-incorporated portion of the present disclosure shall control, and the term or incorporated disclosure therein shall only control with respect to the reference in which the term is defined and/or the incorporated disclosure was present originally.

### Homogeneous Pillar Enhanced Assay (Hope)

FIG. 5 illustrates a schematic view of a homogeneous assay by local concentration according to one embodiment of the present invention. The capture agents are coated on the sidewall of the spacer of a pillar shape.

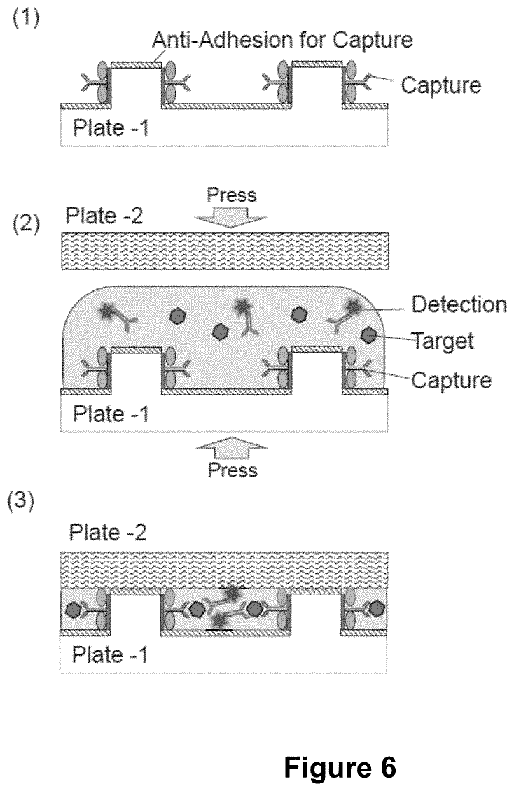

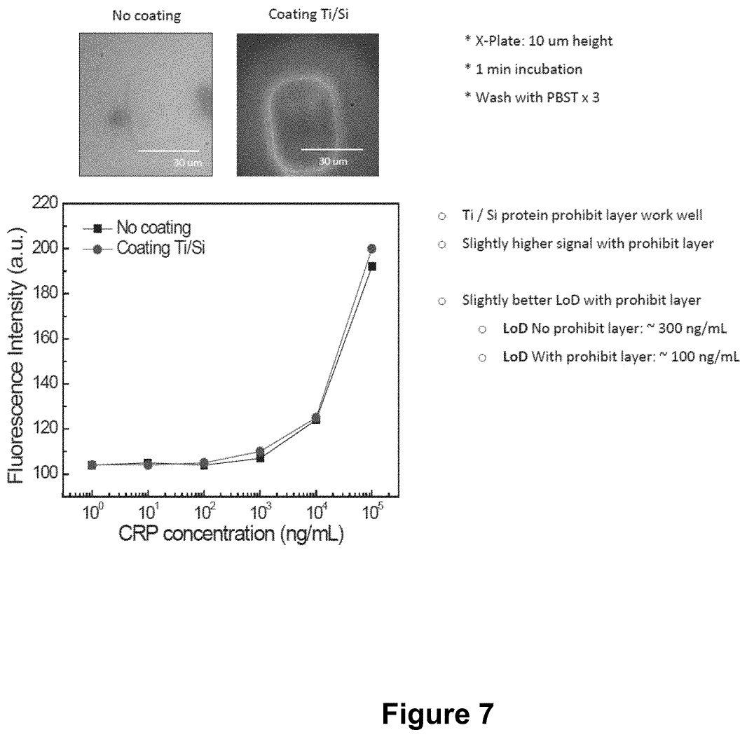

FIG. 6 illustrates a schematic view of a homogeneous assay by local concentration according to one embodiment of the present invention. The capture agents are coated on the sidewall of the spacer of a pillar shape. With a Ti/Si coating on top of the pillar and the surface of the plate, only the pillar sidewall can be coated with capture agent, while without the Ti/Si coating, the capture agent coats everywhere. The images shows that for the capture agent coated only on the sidewall of the spacer gives a stronger fluorescence signal.

### One Example of Detection of CRP Using the Homogeneous Assay by Local Concentration

### Step-1: First Plate as Capture Site:

(1) Prepare 175 um thick X-Plate (Plate 1) with a pillar array of 30×40 um pillar size, 30 um pillar height and 80 um inter spacing distance;
(2) Coat X-Plate with anti-adhesion layer for capture (e.g. 1 nm Ti and 10 nm Si) on top of pillar and bottom of pillar, side wall of pillar is not coated as shown in FIG. 2.
(3) Clean Plate 1 with DI water for 1 minute;
(4) Coat Protein-A/G 10 ug/mL in PBS for 2 h/Wash 3× with PBST;
(5) Coat Capture Ab (goat anti-CRP IgG) 10 ug/mL in PBS coat for 2 h/Wash 3× with PBST; Only side wall of pillar is coated with capture reagents.
   Step-2: Add Antigen, Chamber with Second Plate:
(1) Prepare 100 to 1000 um thick Flat Plate (Plate 2) and clean Plate 2 with DI water for 1 minute;
(2) Pre-coated Plate 2 with detection Ab with a label;
(3) Add the sample with CRP on either Plate 1 or Plate 2;
(4) Close the plate 1 and 2, and press;

### Step-3: Incubation and Detection:

(1) Incubate the sample between plate 1 and 2 for 1 minute;
(2) Imaging the device and analyzing the signal from the label surrounding the pillar region.

### Exemplary Embodiments

1. A method of making a coated well plate, comprising:

a first coating of a well plate having one or more wells to form a first coat layer only on the horizontal surfaces of the well plate, and the vertical surfaces of the well plate are free of the first coat layer; and

a second coating of the vertical surfaces of the well plate with a molecule that can bind an analyte,

wherein:

the first coat is selected from silica, silicate, silicone, silicon, a metal, a metal oxide, and combinations thereof, and the first coating blocks the horizontal surfaces of the well plate from consuming the molecules of in the second coating; and

the molecule of the second coating is a capture agent selected from an antigen, an antibody, and combinations thereof.

2. The method of embodiment 1, wherein the first coating of the well plate is formed by thermal coating, electron beam coating, low pressure chemical vapor depositing coating, or a combination thereof.
3. An article, comprising:

a coated well plate having one or more wells comprising:

a first coat layer only on the horizontal surfaces of a first side of the well plate comprising an anti-capture coat layer; and

a second coat layer only on the vertical surfaces of the well plate, the second coat layer is a capture coat layer having a molecule that can bind an analyte.

4. The article of embodiment 3, wherein:

the anti-capture coat layer is selected from silica, silicate, silicone, silicon, a metal, a metal oxide, and combinations thereof; and

the molecule that can bind an analyte in the capture coat layer is selected from an antibody, an antigen, and combinations thereof.

5. A method of detecting an analyte, comprising:

contacting the article of embodiment 4, with a liquid containing an analyte; and

analyzing at least the vertical surfaces of the well plate for a combination of the capture coat layer and an analyte.

The device, kit, system, or method of any prior claims, wherein the material of the plates is polystyrene, PMMA, PC, COC, COP, or another plastic.

The device, kit, system, or method of any prior claims, wherein the material of the plates is glass.

The device, kit, system, or method of any prior claims, wherein the anti-adhesion layer for capture is metal as aluminum, titanium, silver, gold.

The device, kit, system, or method of any prior claims, wherein the anti-adhesion layer for capture is inorganic material or compound as silicon.

The device, kit, system, or method of any prior claims, wherein the anti-adhesion layer is 1 nm, 2 nm, 3 nm, 5 nm, 10 nm, 50 nm, 70 nm, 100 nm or a range between any two of the values.

The device, kit, system, or method of any prior claims, there is a adhesion layer to adhere the pillar and anti-adhesion layer for capture, wherein the material is Cr or Ti.

The device, kit, system, or method of any prior claims, there is a adhesion layer to adhere the pillar and anti-adhesion layer for capture, wherein the layer has a thickness of 0.1 nm, 0.5 nm, 1 nm, 2 nm, 3 nm, 5 nm, 10 nm or a range between any two of the values.

The device, kit, system, or method of any prior claims, wherein the fabrication of anti-adhesion layer use thermal evaporation process, E-beam evaporation process, LPCVD or ALD.

Wherein the reading device is a CCD camera.

Wherein the reading device is a photodetector comprising one or more other optical devices that are selected from optical filters, spectrometer, lenses, apertures, beam splitter, mirrors, polarizers, waveplates, and shutters.

Wherein the reading device collects the position, local intensity, local spectrum and local Raman signature of said signals.

In some embodiments, the capture antibody can be applied to the surface by printing, spraying, soaking or any other method that applies homogeneous or partial layer of reagents. In certain embodiments, the capture antibody is dried on the first plate. It should also be noted that in some embodiments the capture antibody is coated on the inner surface of the first plate, not the second plate; in some embodiments the capture antibody is coated on the inner surface of the second plate, not the first plate; in some embodiments the capture antibody is coated on the inner surfaces of both plates. In some embodiments, the capture antibody is either monocolonal, polycolonal antibody, engineered antibody (e.g. single chain variable fragments (scFv)) or fragments thereof. In some embodiments, the concentration of coated capture antibody ranges from 1 ng/mL to 1 mg/mL.

In some embodiments, the capture antibody is configured to bind to the analyte. For example, when the analyte comprises an antigen epitope, in certain embodiments the capture antibody is configured to specifically bind to the antigen epitope. In some embodiments, the capture antibody is (a) covalently bound to the surface, or (b) attached to the surface by passive absorption through hydrophobic interactions between solid surface and non-polar residues on the proteins. For example, in some embodiments, the capture antibody is attached to the first plate through protein

In certain embodiments, the capture antibody can immobilize the analyte onto the inner surface of the first plate.

While antibodies can be used to detect antigens, antigens can also be used to detect antibodies. For example, in some embodiments the present invention, a capture antigen (or epitope), instead of the capture antibody, can be coated on the inner surface of a respective plate (e.g. the first plate). The capture antigen can be attached to the inner surface and used to immobilize an analyte (e.g. antibody or antibody-expressing cell) onto the inner surface.

In some embodiments the first plate comprises blockers that are coated on the inner surface of the first plate. In some embodiments, the blockers block any unoccupied sites on the solid surface that can cause unwanted nonspecific bindings in assays. In certain embodiments, the blocker reduces nonspecific binding. In certain embodiments, the blockers can be applied to the surface by printing, spraying, soaking or any other method that applies homogeneous layer of reagents. In certain embodiments, the blockers are dried on the first plate. It should also be noted that in some embodiments the blockers are coated on the inner surface of the first plate, not the second plate; in some embodiments the blockers are coated on the inner surface of the second plate, not the first plate; in some embodiments the blockers are coated on the inner surfaces of both plate. In some embodiments, the blockers are bovine serum albumin (BSA), casein or total proteins from whole milk, etc.

In some embodiments the first plate comprises a stabilizer that is coated on the inner surface of the first plate. In some embodiments, the stabilizer helps maintain the proper folding of protein when dried so that the function of the protein is not disrupted during storage. In certain embodiments, the stabilizer prolongs the usage life span of the reagents, such as but not limited to a protein. In certain embodiments, the stabilizer can be applied to the surface by printing, spraying, soaking or any other method that applies homogeneous layer of reagents. In certain embodiments, the stabilizer is dried on the first plate. It should also be noted that in some embodiments the stabilizer is coated on the inner surface of the first plate, not the second plate; in some embodiments the stabilizer is coated on the inner surface of the second plate, not the first plate; in some embodiments the stabilizer is coated on the inner surfaces of both plates. In some embodiments, the stabilizer is sugar such as but not limited to sucrose and glucose. In some embodiments, the stabilizer is a polymer. In certain embodiments, the stabilizer is glycerol.

In some embodiments the second plate comprises a detection antibody that is coated on the inner surface of the second plate. In some embodiments, the detection antibody can be applied to the surface by printing, spraying, soaking or any other method that applies homogeneous layer of reagents. In certain embodiments, the detection antibody is dried on the second plate. It should also be noted that in some embodiments the detection antibody is coated on the inner surface of the second plate, not the first plate; in some embodiments the detection antibody is coated on the inner surface of the first plate, not the second plate; in some embodiments the detection antibody is coated on the inner surfaces of both plates. In some embodiments, the detection antibody is either monoclonal, polyclonal antibody, engineered antibody (e.g. single chain variable fragments (scFv)) or fragments thereof. In some embodiments, the concentration of coated detection antibody ranges from 1 ng/mL to 1 mg/mL.

In some embodiments, the detection antibody is configured to bind to the analyte. For example, when the analyte comprises an antigen epitope, in certain embodiments the detection antibody is configured to specifically bind to the antigen epitope. In certain embodiments, the capture antibody and the detection antibody bind to different sites (e.g. epitopes) of the analyte. In certain embodiments, the detection antibody is configured to specifically bind to a capture antibody-analyte complex. In certain embodiments, the detection antibody is not covalently bound to the inner surface. In certain embodiments, the detection antibody is not attached to the surface by passive absorption through hydrophobic interactions between solid surface and non-polar residues on the proteins. In certain embodiments, the detection antibody 160 can diffuse into the sample after the sample is deposited and the detection antibody is in contact with the sample liquid.

In some embodiments, the detection antibody is configured to bind to the analyte. For example, when the analyte comprises an antigen epitope, in certain embodiments the detection antibody is configured to specifically bind to the antigen epitope. In certain embodiments, the capture antibody and the detection antibody bind to different sites (e.g. epitopes) of the analyte. In certain embodiments, the detection antibody is configured to specifically bind to a capture antibody-analyte complex. In certain embodiments, the detection antibody is not covalently bound to the inner surface. In certain embodiments, the detection antibody is not attached to the surface by passive absorption through hydrophobic interactions between solid surface and non-polar residues on the proteins. In certain embodiments, the detection antibody 160 can diffuse into the sample after the sample is deposited and the detection antibody is in contact with the sample liquid.

In some embodiments, the detection antibody is configured to produce a detectable signal after binding to the analyte. For example, in some embodiments the signal can be a colorimetric signal, a luminescent signal, or a fluorescent signal. In some embodiments for example, the detection antibody is labeled by a fluorescent label 165, which produces a signal after the detection antibody 1 binds to the analyte or to the capture antibody-analyte complex. In some embodiments, the fluorescent label directly labels the detection antibody. In some embodiments, the fluorescent label 165 labels a reagent that can bind to the detection antibody 160 or a detection antibody-analyte complex. In some embodiments, the secondary antibody can be conjugated with an optical detectable label, e.g., a fluorophore such as but not limited to cy5, IR800, SAPE IRDye800CW, Alexa 790, Dylight 800.

### Assay for Detecting Single Molecule (ADSIM)

### One Example of Detection of CRP Using an Assay for Detecting Single Molecule (ADSiM)

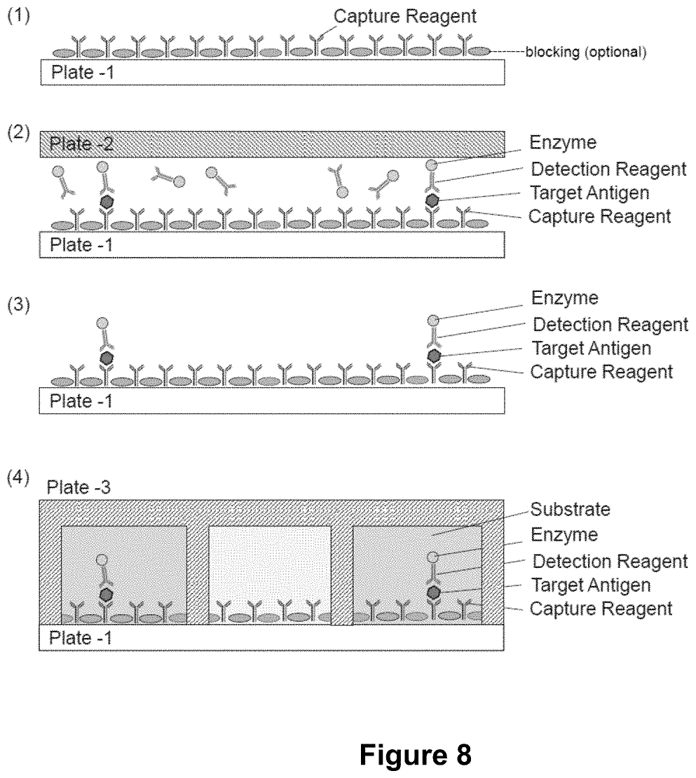

FIG. 8 illustrates a schematic view of an amplification by a single molecule assay according to one embodiment of the present invention.

### Step-1: First Plate as Capture Site:

(1) Clean 1 mm thick glass or acrylic substrate (Plate 1) with DI water for 1 minute;
(2) Coat Protein-A/G 10 ug/mL in PBS for 2 h/Wash 3× with PBST;
(3) Coat Capture Ab (goat anti-CRP IgG) 10 ug/mL in PBS coat for 2 h/Wash 3× with PBST;
   Step-2: Add Antigen with Second Plate:
(1) Prepare 175 um thick X-Plate (Plate 2) with a pillar array of 30×40 um pillar size, 30 um pillar height and 80 um inter spacing distance;
(2) Pre-coated X-Plate with detection Ab with enzyme (mouse anti-CRP IgG with HRP labeling);
(3) Add the sample with CRP on either Plate 1 or Plate 2;
(4) Close the plate 1 and 2, and press;

### Step-3: Incubation and Wash:

(1) Incubate the sample between plate 1 and 2 for 1 minute;
(2) Open the plates;
(3) Wash the plate 1 with Sponge and PBST;

### Step-4: Amplification and Signal Reading:

(1) Prepare 175 um thick Well-Plate (Plate 3) with a well array of 30×30 um well size, 10 um well depth and 20 um inter well distance;
(2) Add the substrate (H₂O₂ and TMB) onto the Plate 3;
(3) Close the plate 1 and 3, and press;
(4) Incubation for 1 min to 30 min;
(5) Imaging the device and counting the well numbers (a) filled with reagents, (b) have the signal.

In some embodiments, the washing steps above can be using washing solution absorbed in a sponge. In some embodiments, the washing is conducted by squeezing the sponge to release the wash solution onto the inner surface of the first plate and releasing the sponge to reabsorb the wash solution. In some embodiments, the washing improves the limit of detection (LOD) for the detectable signal.

In some embodiments, the capture antibody can be applied to the surface by printing, spraying, soaking or any other method that applies homogeneous or partial layer of reagents. In certain embodiments, the capture antibody is dried on the first plate. It should also be noted that in some embodiments the capture antibody is coated on the inner surface of the first plate, not the second plate; in some embodiments the capture antibody is coated on the inner surface of the second plate, not the first plate; in some embodiments the capture antibody is coated on the inner surfaces of both plates. In some embodiments, the capture antibody is either monocolonal, polycolonal antibody, engineered antibody (e.g. single chain variable fragments (scFv)) or fragments thereof. In some embodiments, the concentration of coated capture antibody ranges from 1 ng/mL to 1 mg/mL.

In some embodiments, the capture antibody is configured to bind to the analyte. For example, when the analyte comprises an antigen epitope, in certain embodiments the capture antibody is configured to specifically bind to the antigen epitope. In some embodiments, the capture antibody is (a) covalently bound to the surface, or (b) attached to the surface by passive absorption through hydrophobic interactions between solid surface and non-polar residues on the proteins. For example, in some embodiments, the capture antibody is attached to the first plate through protein A. In certain embodiments, the capture antibody can immobilize the analyte onto the inner surface of the first plate.

While antibodies can be used to detect antigens, antigens can also be used to detect antibodies. For example, in some embodiments the present invention, a capture antigen (or epitope), instead of the capture antibody, can be coated on the inner surface of a respective plate (e.g. the first plate). The capture antigen can be attached to the inner surface and used to immobilize an analyte (e.g. antibody or antibody-expressing cell) onto the inner surface.

In some embodiments the first plate comprises blockers that are coated on the inner surface of the first plate. In some embodiments, the blockers block any unoccupied sites on the solid surface that can cause unwanted nonspecific bindings in assays. In certain embodiments, the blocker reduces nonspecific binding. In certain embodiments, the blockers can be applied to the surface by printing, spraying, soaking or any other method that applies homogeneous layer of reagents. In certain embodiments, the blockers are dried on the first plate. It should also be noted that in some embodiments the blockers are coated on the inner surface of the first plate, not the second plate; in some embodiments the blockers are coated on the inner surface of the second plate, not the first plate; in some embodiments the blockers are coated on the inner surfaces of both plate. In some embodiments, the blockers are bovine serum albumin (BSA), casein or total proteins from whole milk, etc.

In some embodiments the first plate comprises a stabilizer that is coated on the inner surface of the first plate. In some embodiments, the stabilizer helps maintain the proper folding of protein when dried so that the function of the protein is not disrupted during storage. In certain embodiments, the stabilizer prolongs the usage life span of the reagents, such as but not limited to a protein. In certain embodiments, the stabilizer can be applied to the surface by printing, spraying, soaking or any other method that applies homogeneous layer of reagents. In certain embodiments, the stabilizer is dried on the first plate. It should also be noted that in some embodiments the stabilizer is coated on the inner surface of the first plate, not the second plate; in some embodiments the stabilizer is coated on the inner surface of the second plate, not the first plate; in some embodiments the stabilizer is coated on the inner surfaces of both plates. In some embodiments, the stabilizer is sugar such as but not limited to sucrose and glucose. In some embodiments, the stabilizer is a polymer. In certain embodiments, the stabilizer is glycerol.

In some embodiments the second plate comprises a detection antibody that is coated on the inner surface of the second plate. In some embodiments, the detection antibody can be applied to the surface by printing, spraying, soaking or any other method that applies homogeneous layer of reagents. In certain embodiments, the detection antibody is dried on the second plate. It should also be noted that in some embodiments the detection antibody is coated on the inner surface of the second plate, not the first plate; in some embodiments the detection antibody is coated on the inner surface of the first plate, not the second plate; in some embodiments the detection antibody is coated on the inner surfaces of both plates. In some embodiments, the detection antibody is either monoclonal, polyclonal antibody, engineered antibody (e.g. single chain variable fragments (scFv)) or fragments thereof. In some embodiments, the concentration of coated detection antibody ranges from 1 ng/mL to 1 mg/mL.

In some embodiments, the detection antibody is configured to bind to the analyte. For example, when the analyte comprises an antigen epitope, in certain embodiments the detection antibody is configured to specifically bind to the antigen epitope. In certain embodiments, the capture antibody and the detection antibody bind to different sites (e.g., epitopes) of the analyte. In certain embodiments, the detection antibody is configured to specifically bind to a capture antibody-analyte complex. In certain embodiments, the detection antibody is not covalently bound to the inner surface. In certain embodiments, the detection antibody is not attached to the surface by passive absorption through hydrophobic interactions between solid surface and non-polar residues on the proteins. In certain embodiments, the detection antibody 160 can diffuse into the sample after the sample is deposited and the detection antibody is in contact with the sample liquid.

In some embodiments, the detection antibody is configured to bind to the analyte. For example, when the analyte comprises an antigen epitope, in certain embodiments the detection antibody is configured to specifically bind to the antigen epitope. In certain embodiments, the capture antibody and the detection antibody bind to different sites (e.g. epitopes) of the analyte. In certain embodiments, the detection antibody is configured to specifically bind to a capture antibody-analyte complex. In certain embodiments, the detection antibody is not covalently bound to the inner surface. In certain embodiments, the detection antibody is not attached to the surface by passive absorption through hydrophobic interactions between solid surface and non-polar residues on the proteins. In certain embodiments, the detection antibody 160 can diffuse into the sample after the sample is deposited and the detection antibody is in contact with the sample liquid.

In some embodiments, the detection antibody is configured to produce a detectable signal after binding to the analyte. For example, in some embodiments the signal can be a colorimetric signal, a luminescent signal, or a fluorescent signal. In some embodiments for example, the detection antibody is labeled by a fluorescent label 165, which produces a signal after the detection antibody 1 binds to the analyte or to the capture antibody-analyte complex. In some embodiments, the fluorescent label directly labels the detection antibody. In some embodiments, the fluorescent label 165 labels a reagent that can bind to the detection antibody 160 or a detection antibody-analyte complex. In some embodiments, the secondary antibody can be conjugated with an optical detectable label, e.g., a fluorophore such as but not limited to cy5, IR800, SAPE IRDye800CW, Alexa 790, Dylight 800.

In some embodiments, the detection antibody is configured to a chemical that can amplified signal or the signal from this chemical can be amplified; wherein amplification method in this amplification step including, but not limit to:

The color based enzymatic reaction, the absorption signal generated by substrates are amplified by enzyme which are linked to the detection reagents; wherein the enzyme including horseradish peroxidase; wherein the substrates including ABTS or TMB;

The fluorescence based enzymatic reaction, the fluorescence signal generated by substrates are amplified by enzyme which are linked to the detection reagents; wherein the enzyme including horseradish peroxidase or β-galactosidase; wherein the substrates including Amplex red or Resorufin β-D-Galactopyranoside;

Catalytic amplification. An analyte activates a catalyst, which then produces multiple copies of a reporter molecule.

Catalytic self-amplification. An analyte activates a catalyst, which results in the production of reporter molecules. These not only generate a signal, but are also able to activate the catalyst.

Analyte-induced modification of a collective property. The binding of a single analyte molecule to a receptor affects the properties of neighboring units through signal transduction.

Multivalent surfaces for binding of multiple analyte molecules. Recruitment of multiple reporters using multivalent scaffolds such as polymers, dendrimers or nanoparticles amplifies the signal.

Wherein above catalysts including Pd(0)-catalyst, apyrase, potassium permanganate, platinum, etc.

While antibodies can be used to detect antigens, antigens can also be used to detect antibodies. For example, in some embodiments of the present invention, a detection antigen (or epitope), instead of the detection antibody, can be coated on the inner surface of a respective plate (e.g. the second plate). The capture antigen can be attached to the inner surface and used to detect an analyte (e.g. antibody or antibody-expressing cell) onto the inner surface.

### Exemplary Embodiments

1. A method for an assay that detects a single molecule in a sample, comprising: immobilizing a capture agent on a first surface of a first plate;
depositing an enzyme-linked detection agent on a first surface of a second plate, the enzyme-linked detection agent capable of diffusing in the sample when contacting the sample;
depositing a substrate on a third plate having a first side with one or more microwells;
depositing a sample suspected of containing an analyte on the first plate or the second plate;
contacting the first surfaces of the first plate and the second plate to contact the capture agent and the enzyme-linked detection agent with the sample;
incubating the first plate and the second plate while contacted for a first period of time to form an enzymatic complex if analyte is present, the enzymatic complex including the capture agent and the enzyme-linked detection agent bound to the analyte;
separating the first plate and second plate after the first period of time;
washing the first surface of the first plate;
contacting the washed first surface of the first plate with the first side of the third plate;
compressing the first plate and the third plate together;
incubating the first plate and the third plate while contacted for a second period of time to contact the enzymatic complex with the substrate in the microwells and generate a signal;
imaging the signal; and
digitally analyzing the microwells of the third plate that have a signal;
wherein:
the capture agent is selected to bind specifically to the analyte if the analyte is present in the sample;
the enzyme-linked detection agent is selected to bind specifically to the analyte if the analyte is present in the sample;
the enzymatic complex will form if an analyte is present in the sample;
washing removes unbound enzyme-linked detection agent; and
the enzyme-linked detection agent causes the substrate to generate the signal.
   2. A method for an assay that detects a single molecule in a sample, comprising: providing a first plate;
providing a second plate
providing a third plate including a first side having one or more microwells;
immobilizing a capture agent on a first surface of the first plate;
depositing an enzyme-linked detection agent on a first surface of the second plate, the enzyme-linked detection agent capable of diffusing in the sample when contacting the sample;
depositing a substrate in the one or more microwells;
depositing a sample suspected of containing an analyte on either of the first surfaces of the first plate or the second plate;
contacting the first surfaces of the first plate and the second plate to contact the capture agent and the enzyme-linked detection agent with the sample;
incubating the first plate and the second plate while contacted for a first period of time to form an enzymatic complex if analyte is present, the enzymatic complex including the capture agent and the enzyme-linked detection agent bound to the analyte;
separating the first plate and second plate after the first period of time;
washing the first surface of the first plate;
contacting the washed first surface of the first plate with the first side of the third plate; compressing the first plate and the third plate together;
incubating the first plate and the third plate while contacted for a second period of time to contact the enzymatic complex with the substrate and generate a signal;
imaging the signal; and
digitally analyzing the microwells of the third plate that have a signal;
wherein:
the capture agent is selected to bind specifically to the analyte if the analyte is present in the sample;
the enzyme-linked detection agent is selected to bind specifically to the analyte if the analyte is present in the sample;
the enzymatic complex will form if an analyte is present in the sample;
washing removes unbound enzyme-linked detection agent; and
the enzyme-linked detection agent causes the substrate to generate the signal.
   3. An apparatus, comprising:
a first plate including a capture agent immobilized on a surface of the first plate;
a second plate including an enzyme-linked detection agent deposited on a surface of the second plate;
a third plate including one or more microwells having a substrate to provide a signal;
one or more spacers disposed on the first plate or the second plate;
wherein:
the first plate and second plate are movable relative to each other between a first open configuration and a first closed configuration;
the first plate and third plate are movable relative to each other between a second open configuration and a second closed configuration;
in the first open configuration a sample suspected of containing an analyte is deposited onto either or both of the first plate or the second plate;
in the first closed configuration the capture agent and the enzyme-linked detection agent contact the sample forming an enzymatic complex if the analyte is present, the enzymatic complex including the capture agent and the enzyme-linked detection agent bound to the analyte; and
in the second closed configuration at least part of the enzymatic complex is positioned in the one or more wells to contact the substrate.
   4. The apparatus of embodiment 3, wherein:
the one or more spacers include a pillar shape, a substantially flat top surface, a predetermined substantially uniform height and a predetermined inter-spacer distance;
the inter-spacer distance is a distance between two neighboring spacers;
a Young's modulus of the spacer multiplied by the filling factor of the spacer is equal to or larger than 2 MPa; and
the filling factor is the ratio of a spacer contact area to a total sample contact area of the plate.
   5. The apparatus of embodiment 3, further comprising an imager to image the signal.
   6. The apparatus of embodiment 3, an analyzer for digitally analyzing the signal.

What is claimed is:

1. A method for performing a homogeneous assay of an analyte in a sample, comprising:
(a) providing a sample that contains or is suspected of containing an analyte;
(b) providing one bead;
(c) providing a capture agent and a labeled detection agent;
(d) providing a sample holder that is configured to make at least a part of the sample into a sample layer of a thickness of 200 um or less;
(e) having the sample in the sample holder, wherein the bead and the labeled detection agent are mixed with the sample in the sample layer;
(f) taking, after step (e), without washing the sample, at least two images, including a first image and a second image, of a common area of the sample layer, wherein the common area of the sample layer is an area of the sample that contains the bead, wherein the first image is a direct image for measuring topology of the sample including a position and geometry of the bead; and the second image is a signal image for measuring a signal from the labeled detection agent;
(g) after (f), comparing and analyzing, using an algorithm, the first image and the second image to identify a signal from a labeled detection agent that is attached to the bead;
wherein the capture agent is attached onto the surface of the bead, and binds to the analyte or the labeled detection agent; and
wherein the labeled detection agent binds to the capture agent or the analyte.

2. An apparatus for performing a homogeneous assay of an analyte in a sample, comprising:
(a) a bead
(b) a capture agent;
(c) a labeled detection agent;
(d) a sample holder that is configured to make at least a part of the sample into a sample layer of a thickness of 200 um or less;
(f) an imager that is configured to take at least two images, including a first image and a second image, of a common area of the sample layer, wherein the common area of the sample layer is an area of the sample that contains the bead, wherein the first image is a direct image for measuring topology of the sample including a position and geometry of the bead; and the second image is a signal image for measuring a signal from the labeled detection agent; and
(g) a computer readable medium that contain an algorithm that compares and analyzes, using an algorithm, the first image and the second image to identify a signal from a labeled detection agent that is attached to the bead;
wherein the capture agent is attached onto the surface of the bead, and bines to the analyte or the labeled detection agent; and
wherein the labeled detection agent gives a signal and binds to the capture agent or the analyte.

3. The apparatus and method of any claim, wherein the thickness of the sample layer and the diameter of the bead are selected, so that when there are more than one beads, the beads do not substantially overlap with each other in a direction normal to the sample layer such that when viewing from the top of the sample layer, no bead substantially blocks a view of any other bead.

4. The apparatus and method of any prior claim, wherein the first image is bright field image.

5. The apparatus and method of any prior claim, wherein the second image is a fluorescence and/or other luminescence image.

6. The apparatus and method of any prior claim, wherein the second image is a dark field image.

7. The apparatus and method of any prior claim, wherein the signal is an optical signal.

8. The apparatus and method of any prior claim, wherein the capture agent binds only to the analyte, and the labeled detection agent binds only to the analyte.

9. The apparatus and method of any prior claim, wherein the capture agent binds to both the analyte and the labeled detection agent, and the labeled detection agent binds only the capture agent.

10. The apparatus and method of any prior claim, wherein the capture agent binds to both the labeled detection agent, and the labeled detection agent binds to both the analyte and the capture agent.

11. The apparatus and method of any prior claim, wherein the algorithm use an image of the spacer in the first image and/or the second image.

12. The apparatus and method of any prior claim, wherein the label detection agent has a label that is selected from the group consisting of a fluorescent label, a colorimetric label, and luminescent label.

13. The apparatus and method of any claim, wherein the beads have various shape and have a maximum dimension in the range of 0.05 um to 50 um.

14. The apparatus and method of any claim; wherein the sample holder is configured make the sample layer having uniform thickness.

15. The apparatus and method of any claim, wherein sample holder comprising a first plate and a second plate that are movable relative to each other into different configurations, including an operation and a closed configuration;
wherein in the open configuration the first plate and second plate are at least partially separated such that the sample can be deposited on one or both plates; and
wherein the closed configuration is configured after the sample deposition in the open configuration, and in the closed configuration: the first plate and the second plate confine at least a portion of the sample between the plates into a layer having a thickness of 200 um or less.

16. The apparatus and method of any prior claim, wherein sample holder comprising:
a. a first plate and a second plate that are movable relative to each other into different configurations, including an operation and a closed configuration;
b. one or both of plates are flexible; and
c. spacers that have a uniform height of 200 um or less, and are fixed on one of the plates;
wherein in the open configuration the first plate and second plate are at least partially separated and the spacing between the two plate are not regulated by the spacers, such that the sample can be deposited t on one or both plates; and
wherein the closed configuration is configured after the sample deposition in the open configuration, and in the closed configuration: at least part of the deposited sample is confined by the two plates into a thin layer that has a substantially uniform thickness, the substantially uniform thickness is regulated by the plates and the spacers.

17. A kit for performing a homogeneous assay for analyzing an analyte in a sample, comprising:
(c) a bead
(d) a capture agent;
(c) a labeled detection agent;
(d) a sample holder that is configured to make at least a part of the sample into a sample layer of a thickness of 200 um or less;
wherein the capture agent is attached onto the surface of the bead, and bines to the analyte or the labeled detection agent; and
wherein the labeled detection agent gives a signal and binds to the capture agent or the analyte;
wherein sample holder comprising a first plate and a second plate that are movable relative to each other into different configurations, including an operation and a closed configuration;
wherein in the open configuration the first plate and second plate are at least partially separated such that the sample can be deposited on one or both plates; and
wherein the closed configuration is configured after the sample deposition in the open configuration, and in the closed configuration: the first plate and the second plate confine at least a portion of the sample between the plates into a layer having a thickness of 200 um or less.

18. A programed imager for performing a homogeneous assay for analyzing an analyte in a sample, comprising:
an imager that is configured to take at least two images, including a first image and a second image, of a common area of the sample layer, wherein the common area of the sample layer is an area of the sample that contains the bead, wherein the first image is a direct image for measuring topology of the sample including a position and geometry of the bead; and the second image is a signal image for measuring a signal from the labeled detection agent; and
a computer readable medium that contain an algorithm that compares and analyzes, using an algorithm, the first image and the second image to identify a signal from a labeled detection agent that is attached to the bead;
wherein the capture agent is attached onto the surface of the bead, and bines to the analyte or the labeled detection agent; and
wherein the labeled detection agent gives a signal and binds to the capture agent or the analyte.

19. The apparatus and method of any prior claim, wherein the first image is bright field image.

20. The apparatus and method of any prior claim, wherein the second image is a dark field image.

21. The apparatus and method of any prior claim, wherein the second image is a dark field image, and the signal is a fluorescence and/or other luminescence signal.

22. The apparatus and method of any prior claim, wherein the signal is an optical signal.

23. The apparatus and method of any prior claim, wherein the labeled detection agent binds to the analyte, but not to the capture agent.

24. The apparatus and method of any prior claim, wherein the labeled detection agent binds the capture agent, but not to the analyte.

25. The apparatus and method of any prior claim, wherein the algorithm use an image of the spacer in the first image and/or the second image.

26. The apparatus and method of any prior claim, wherein the label detection agent has a label that is selected from the group consisting of a fluorescent label, a colorimetric label, and luminescent label.

27. The apparatus and method of any prior claim, wherein the algorithm is machine learning.

28. The apparatus and method of any prior claim, wherein the algorithm is machine learning and wherein the machine learning utilizes a property of the spacers.

29. The apparatus and method of any prior claim, wherein the algorithm is machine learning and wherein the machine learning utilizes a property of the beads.

30. The apparatus and method of any prior claim, wherein the algorithm is machine learning and wherein the machine learning analyze air bubble, dust, breakage, other non-sample factors or any combination in the sample layer.

31. The apparatus and method of any prior claim, wherein the bead comprising more than one beads, wherein the beads are arranged to make the beads not substantially overlapping with each other in a direction normal to the sample layer, such that when viewing from the top of the sample layer, no bead substantially blocks a view of any other bead.

32. The apparatus and method of any prior claim, wherein the thickness of the sample layer and the concentration of the labeled detection agent are selected, so that the labeled detection agent attached to the capture agent on the bead is distinguishable from signal emanating from other area in the layer of uniform thickness.

33. The apparatus and method of any prior claim, wherein in an open configuration, the beads are on the same plate that the spacers are fixed.

34. The apparatus and method of any prior claim, wherein the spacer height is the same as the maximum size of a bead (e.g. diameter) and is 15 um or less.

35. The apparatus and method of any prior claim, wherein the spacer height is the same as the maximum size of a bead (e.g. diameter) and is 10 um.

36. The apparatus and method of any prior claim further comprising a second set of capture agent and labeled detection agent, wherein the second capture agent is attached on the bead and captures a second analyte in the sample or the second labeled detection agent, and the second labeled detection agent binds to the second capture agent or the second analyte, and wherein the second analyte is bio/chemically different analyte from the first analyte in the sample.

37. The apparatus and method of any prior claim further comprising more than one set of capture agent and labeled detection agent, wherein each set of capture agent is attached on the bead and captures a corresponding analyte in the sample or the labeled detection agent, and each set of labeled detection agent binds to the corresponding capture agent or the corresponding analyte, and wherein each set of analyte is bio/chemically different analyte from other set of analyte in the sample.

38. The apparatus and method of any prior claim further comprising a second set of capture agent and labeled detection agent, and a second set of bead, wherein the second capture agent is attached on the second set of bead and captures a second analyte in the sample or the second set of labeled detection agent, and the second labeled detection agent binds to the second capture agent or the second analyte, and wherein the second analyte is bio/chemically different analyte from the first analyte in the sample and the second set of bead has a different property from the first set of beads.

39. The apparatus and method of any prior claim further comprising more than one set of capture agent and labeled detection agent, and more than one set of beads, wherein each set of capture agent is attached on each corresponding set of bead and captures a corresponding analyte in the sample or the labeled detection agent, and each set of labeled detection agent binds to the corresponding capture agent or the corresponding analyte, and wherein each set of analyte is bio/chemically different analyte from other set of analyte in the sample, and each set of bead has a different property from other set of beads.

40. The apparatus and method of any prior claim further comprising more than one set of capture agent and labeled detection agent, wherein each set of capture agent is attached on the bead and captures a corresponding analyte in the sample or the labeled detection agent, wherein at least one set of labeled detection agent binds only to the corresponding analyte, and wherein each set of analyte is bio/chemically different analyte from other set of analyte in the sample.

41. The apparatus and method of any prior claim further comprising more than one set of capture agent and labeled detection agent, and more than one set of beads, wherein each set of capture agent is attached on each corresponding set of bead and captures a corresponding analyte in the sample or the labeled detection agent, wherein at least one set of labeled capture agent binds only to the corresponding set of labeled detection agent, and wherein each set of analyte is bio/chemically different analyte from other set of analyte in the sample, and each set of bead has a different property from other set of beads.

42. The apparatus and method of any prior claim, wherein the apparatus and methods comprising a combination of all prior claims.

43. The apparatus and method of any prior claim, wherein different set of the labeled detection agent has a different property respect each other.

44. The apparatus and method of any prior claim, wherein different set of the labeled detection agent has a different property respect each other, including different optical spectrum.

45. The apparatus and method of any prior claim, wherein the capture agent comprises a molecule, protein, nucleic acid, or aptamer.

46. The apparatus and method of any prior claim, wherein the labeled detection agent comprises a molecule, protein, nucleic acid, or aptamer.

47. The apparatus and method of any prior claim, wherein the analyte amount in the sample is determined from the total amplitude of the light from all beads in the measurement area.

48. The apparatus and method of any prior claim, wherein the analyte amount in the sample is determined from the number of the beads that have a light signal above a threshold value, wherein the threshold value is determined from a calibration and wherein as long as the light from a bead is equal or above the threshold it counts one bead regardless how much it is above the threshold.

49. The apparatus and method of any prior claim, wherein the concentration of the analyte is measured by measuring the signal on the bead(s).

50. The apparatus and method of any prior claim, wherein the concentration of the analyte is measured by measuring the signal on the bead(s) and measuring the signal in the sample layer but away from the bead(s).

51. The apparatus and method of any prior claim, wherein the beads have a capture agent attached on their surface and have a maximum size of 0.2 um to 100 um;

52. The apparatus and method of any prior claim, wherein an algorithm to identify the signal at the beads.

53. The apparatus and method of any prior claim, wherein, in the thin sample layer, the beads are randomly distributed.

54. The apparatus and method of any prior claim, wherein the total assay time is less than 10 sec, 20 sec, 30 sec, 40 sec, 50 sec, 60 sec, 120 sec, 180 sec, 240 sec, 300 sec, 400 sec, or 500 sec.

55. The apparatus and method of any prior claim, wherein the beads have a diameter in a range of 1 μm to 10 μm, or 10 μm to 50 μm.

56. The apparatus and method of any prior claim, wherein the beads or beads can be made of polystyrene, polypropylene, polycarbonate, glass, metal or any other material whose surface can be modified to bind antibodies.

57. The apparatus and method of any prior claim, wherein the diameter of the beads is no larger than the pillar height.

58. The apparatus and method of any prior claim, wherein the diameter of the beads about the same as the pillar height.

59. A smartphone system for homogeneous assay, comprising:
(a) a device of any prior claim;
(b) a mobile communication device that comprises:
i. one or a plurality of cameras for detecting and/or imaging the sample;
ii. electronics, signal processors, hardware and software for receiving and/or processing the detected signal and/or the image(s) of the sample and for remote communication; and
(c) an adaptor that is configured to accommodate the device that is in the closed configuration and be engageable to the mobile communication device;
wherein when engaged with the mobile communication device, the adaptor is configured to facilitate the detection and/or imaging of the analyte in the sample; and
wherein the imager takes, at least two images, including a first image and a second image, of a common area of the thin sample layer, wherein the common area of the thin sample layer is an area of the sample that contains at least one bead, wherein the first image is a direct image for measuring a position of a bead in the common area; and the second image is a signal image for measuring a signal from the labeled competitive detection agent.

60. The apparatus and method of any prior claim, wherein the first image and the second image, each comprises multiple images.

61. The apparatus and method of any prior claim, wherein the spacer or the beads are arranged periodically.

62. The apparatus and method of any prior claim, wherein the first and second beads are different in their optical properties selected from the group consisting of: photoluminescence, electroluminescence, and electrochemiluminescence, light absorption, reflection, transmission, diffraction, scattering, diffusion, surface Raman scattering, and any combination thereof.

63. The apparatus and method of any prior claim, wherein the labeled detection agent is coated on one or both of the plates, and is configured to, upon contacting the sample, be dissolved and diffuse in the sample.

64. The apparatus and method of any prior claim, wherein the labeled detection agent is pre-loaded into the sample before the sample is deposited on the plate(s).

65. The apparatus and method of any prior claim, wherein the beads have an average diameter in the range of 0.1 μm to 10 μm.

66. The apparatus and method of any prior claim, wherein the analyte is selected from the group consisting of: molecules, cells, viruses, proteins, peptides, DNAs, RNAs, nucleic acid, nanoparticles, and any combination thereof.

67. The apparatus and method of any prior claim, wherein the capture agent is a protein.

68. The apparatus and method of any prior claim, wherein the capture agent is a nucleic acid.

69. The apparatus and method of any prior claim, wherein the labeled detection agent is a protein.

70. The apparatus and method of any prior claim, wherein the labeled detection agent is a nucleic acid.

71. The apparatus and method of any prior claim, wherein the liquid sample is made from a biological sample selected from the group consisting of: amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, urine, and any combination thereof.

72. The apparatus and method of any prior claim, wherein the sample is an environmental liquid sample from a source selected from the group consisting of: river, lake, pond, ocean, glaciers, icebergs, rain, snow, sewage, reservoirs, tap water, or drinking water, solid samples from soil, compost, sand, rocks, concrete, wood, brick, sewage, and any combination thereof.

73. The apparatus and method of any prior claim, wherein the sample is an environmental gaseous sample from a source selected from the group consisting of: the air, underwater heat vents, industrial exhaust, vehicular exhaust, and any combination thereof.

74. The apparatus and method of any prior claim, wherein the sample is a foodstuff sample selected from the group consisting of: raw ingredients, cooked food, plant and animal sources of food, preprocessed food, and partially or fully processed food, and any combination thereof.

75. The apparatus and method of any prior claim, wherein the detection agent is labeled with a fluorophore.

76. The apparatus and method of any prior claim, wherein the beads are associated with a label, and wherein the detection agent is a quencher that is configured to quench signal of the beads-associated label when the detection agent is in proximity of the label.

77. The apparatus and method of any prior claim, wherein the signal is:
i. luminescence selected from the group consisting of photoluminescence, electroluminescence, and electrochemiluminescence;
ii. light absorption, reflection, transmission, diffraction, scattering, or diffusion;
iii. surface Raman scattering; and
vi. any combination of i-vi.

78. The method of any prior claim, further comprising determining the presence of the analyte and/or measuring the amount of the analyte.

79. The apparatus and method of any prior claim, wherein the one or more beads have a maximum dimension in the range of 0.05 um to 30 um.

80. wherein the thickness of the sample is 0.1 um, 0.5 um, 1 um, 2 um, 3 um, 4 um, 5 um, 10 um, 15 um, 20 um, 25 um, 30 um, 50 um, or a range between any two values thereof.

81. The apparatus and method of any prior claim, wherein the spacer height is equal to the diameter of the beads.
